(19) United States
(12) Patent Application Publication
(10) Pub. No.: US 2022/0102637 A1
(43) Pub. Date: Mar. 31, 2022

(54) PYRROMETHENE METAL COMPLEX, PYRROMETHENE COMPOUND, LIGHT-EMITTING ELEMENT MATERIAL, LIGHT-EMITTING ELEMENT, DISPLAY DEVICE, AND ILLUMINATION DEVICE

(71) Applicant: Toray Industries, Inc., Tokyo (JP)

(72) Inventors: Kazunari Kawamoto, Otsu-shi, Shiga (JP); Takashi Tokuda, Otsu-shi, Shiga (JP); Kazumasa Nagao, Otsu-shi, Shiga (JP)

(73) Assignee: Toray Industries, Inc., Tokyo (JP)

(21) Appl. No.: 17/434,809

(22) PCT Filed: Mar. 5, 2020
(86) PCT No.: PCT/JP2020/009363

§ 371 Date: Aug. 30, 2021

(30) Foreign Application Priority Data
Mar. 11, 2019 (JP) 2019-043435
Dec. 17, 2019 (JP) 2019-227031

(51) Int. Cl. H01L 51/00; C07F 5/02; C09K 11/06

(52) U.S. Cl. CPC H01L 51/008; H01L 51/5012; C09K 11/06; C07F 5/027

(57) ABSTRACT

An object of the present invention is to provide a red-light-emitting material and a red-light-emitting element that have high luminous efficiency and excellent color purity. The present invention is a pyrromethene metal complex represented by a specific general formula.

### CROSS REFERENCE TO RELATED APPLICATIONS

This is the U.S. National Phase application of PCT/JP2020/009363, filed Mar. 5, 2020, which claims priority to Japanese Patent Application No. 2019-043435, filed Mar. 11, 2019 and Japanese Patent Application No. 2019-227031, filed Dec. 17, 2019, the disclosures of each of these applications being incorporated herein by reference in their entireties for all purposes.

### FIELD OF THE INVENTION

The present invention relates to a pyrromethene metal complex, a pyrromethene compound, a light-emitting element material, a light-emitting element, a display device, and an illumination device.

### BACKGROUND OF THE INVENTION

In an organic thin-film light-emitting element, electrons injected from a cathode and holes injected from an anode are recombined in an emissive layer sandwiched between both the electrodes to emit light. The organic thin-film light-emitting element has characteristics of being thin, driven at a low driving voltage, capable of achieving high luminance light emission, and capable of achieving multicolor light emission through selection of an emissive material.

Among multicolor light emission, red-light emission has been studied as emission with a useful light emission color. Examples of a conventionally known red-light-emitting material include perylene-based materials such as bis(diisopropylphenyl)perylene, perinone-based materials, tetracene-based materials, porphyrin-based materials, and Eu complexes (Chem. Lett., 1267 (1991)).

As a method for obtaining red-light emission, a method has also been studied in which a trace amount of a red fluorescent material as a dopant is mixed into a host material. Examples of the dopant material particularly include materials containing a pyrromethene metal complex that exhibits high luminance emission (see, for example, Patent Document 1). Furthermore, a compound is also known in which a condensed ring structure is introduced into a pyrromethene skeleton in order to obtain a sharp emission spectrum (see, for example, Patent Document 2). In recent years, a light-emitting element including a thermally activated delayed fluorescence (TADF) material and a pyrromethene compound has been studied for high luminous efficiency (see, for example, Patent Document 3).

### PATENT DOCUMENTS

Patent Document 1: Japanese Patent Laid-open Publication No. 2003-12676
Patent Document 2: Japanese Patent Laid-open Publication No. 2002-134275
Patent Document 3: International Publication No. 2016/056559

### SUMMARY OF THE INVENTION

In the case that an organic thin-film light-emitting element is used as a display device or an illumination device, the color gamut is to be widen. The color gamut is represented by a triangle obtained by connecting vertex coordinates determined to indicate emission of red, green, and blue light respectively in an xy chromaticity diagram. In order to widen the color gamut, the vertex coordinate of each of red, green, and blue is to be set to appropriate chromaticity so as to widen the area of the triangle, and various color designs have been performed.

The chromaticity depends on the combination of the light emission peak wavelength and the color purity. The color purity depends on the width of the emission spectrum, and the smaller the width is to be similar to the width of monochromatic light, the higher the color purity is. In order to widen the color gamut, increase in the color purity is particularly important, and an emissive material having a sharp emission spectrum has been strongly awaited.

Meanwhile, from the viewpoints of improving luminance and saving power, an organic thin-film light-emitting element having high luminous efficiency has been awaited. In particular, in mobile display devices that have been increasingly used in recent years, power saving is a particularly important problem.

Under such circumstances, it has been difficult to achieve both high luminous efficiency and high color purity in the red-light-emitting materials used in the prior art. The emissive material in which a condensed ring structure is introduced into a pyrromethene skeleton as described in Patent Document 2 has a problem that color design for appropriate chromaticity is difficult because, although such an emissive material has good color purity, the light emission peak wavelength derived from the basic skeleton is so long that control of the wavelength is difficult.

An object of the present invention is to solve the problems of the prior art, and to provide a red-light-emitting material and a light-emitting element in which the luminous efficiency and the color purity are high and the color design for appropriate chromaticity is easy.

The present invention according to exemplary embodiments is a pyrromethene metal complex represented by a general formula (1) or (2).

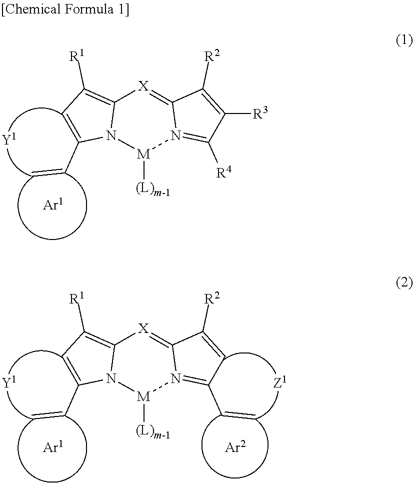

X represents C—R⁵ or N.

R¹ to R⁵ are the same or different from each other, and are each selected from a hydrogen atom, an alkyl group, a cycloalkyl group, a heterocyclic group, an alkenyl group, a cycloalkenyl group, an alkynyl group, an aryl group, a heteroaryl group, a hydroxyl group, a thiol group, an alkoxy group, an alkylthio group, an aryl ether group, an aryl thioether group, a halogen, a cyano group, an aldehyde group, an acyl group, a carboxyl group, an ester group, an amide group, a sulfonyl group, a sulfonic acid ester group, a sulfonamide group, an amino group, a nitro group, a silyl group, a siloxanyl group, a boryl group, a phosphine oxide group, and a ring structure with an adjacent group, and the functional groups that are substituted, a ring structure formed by R³ and R⁴ is a monocyclic ring, R⁴ is not a hydrogen atom and not a halogen in a case that Y⁴ is a trimethylene group.

Ar¹ and Ar² are the same or different from each other, and are each selected from an aromatic hydrocarbon ring bonded to a substituent or no substituent and an aromatic heterocyclic ring bonded to a substituent or no substituent.

Y⁴ is a bridging structure in which three or more atoms are bonded in series and form a double bond or no double bond with an adjacent atom, and the three or more atoms are selected from a carbon atom bonded to a substituent or no substituent, a silicon atom bonded to a substituent or no substituent, a nitrogen atom bonded to a substituent or no substituent, a phosphorus atom bonded to a substituent or no substituent, an oxygen atom, and a sulfur atom.

Z¹ is a bridging structure in which one or more atoms are bonded and form a double bond or no double bond with an adjacent atom, and the one or more atoms are selected from a carbon atom bonded to a substituent or no substituent, a silicon atom bonded to a substituent or no substituent, a nitrogen atom bonded to a substituent or no substituent, a phosphorus atom bonded to a substituent or no substituent, an oxygen atom, and a sulfur atom.

M represents a metal having a valence of m, and is at least one selected from boron, beryllium, magnesium, zinc, chromium, iron, cobalt, nickel, copper, manganese, and platinum.

L is the same or different from each other, and are each selected from an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, and a cyano group, and the functional groups that are substituted.

According to the present invention, it is possible to obtain a red-light-emitting material and a light-emitting element in which the luminous efficiency and the color purity are high and the color design for appropriate chromaticity is easy.

### DETAILED DESCRIPTION OF EMBODIMENTS OF THE INVENTION

Hereinafter, preferred embodiments of the pyrromethene metal complex according to the present invention, and a light-emitting element material, a light-emitting element, a display device, and an illumination device that include the pyrromethene metal complex will be described in detail. However, the present invention is not limited to the following embodiments, and various modifications can be made according to the purpose and the use.

<Pyrromethene Metal Complex>

The pyrromethene metal complex according to embodiments of the present invention is represented by a general formula (1) or (2).

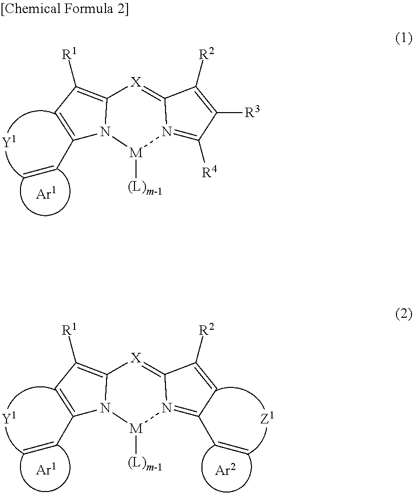

X represents C—R⁵ or N.

R¹ to R⁵ are the same or different from each other, and are each selected from a hydrogen atom, an alkyl group, a cycloalkyl group, a heterocyclic group, an alkenyl group, a cycloalkenyl group, an alkynyl group, an aryl group, a heteroaryl group, a hydroxyl group, a thiol group, an alkoxy group, an alkylthio group, an aryl ether group, an aryl thioether group, a halogen, a cyano group, an aldehyde group, an acyl group, a carboxyl group, an ester group, an amide group, a sulfonyl group, a sulfonic acid ester group, a sulfonamide group, an amino group, a nitro group, a silyl group, a siloxanyl group, a boryl group, a phosphine oxide group, and a ring structure with an adjacent group, and the functional groups that are substituted, a ring structure formed by R³ and R⁴ is a monocyclic ring, R¹ is not a hydrogen atom and not a halogen in a case that Y¹ is a trimethylene group.

Ar¹ and Ar² are the same or different from each other, and are each selected from an aromatic hydrocarbon ring bonded to a substituent or no substituent and an aromatic heterocyclic ring bonded to a substituent or no substituent.

Y¹ is a bridging structure in which three or more atoms are bonded in series and form a double bond or no double bond with an adjacent atom, and the three or more atoms are selected from a carbon atom bonded to a substituent or no substituent, a silicon atom bonded to a substituent or no substituent, a nitrogen atom bonded to a substituent or no substituent, a phosphorus atom bonded to a substituent or no substituent, an oxygen atom, and a sulfur atom.

Z¹ is a bridging structure in which one or more atoms are bonded and form a double bond or no double bond with an adjacent atom, and the one or more atoms are selected from a carbon atom bonded to a substituent or no substituent, a silicon atom bonded to a substituent or no substituent, a nitrogen atom bonded to a substituent or no substituent, a phosphorus atom bonded to a substituent or no substituent, an oxygen atom, and a sulfur atom.

M represents a metal having a valence of m, and is at least one selected from boron, beryllium, magnesium, zinc, chromium, iron, cobalt, nickel, copper, manganese, and platinum.

L is the same or different from each other, and are each selected from an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, and a cyano group, and the functional groups that are substituted.

In the present invention, the term “pyrromethene” refers to compounds having a pyrromethene skeleton represented by the general formula (3) in which X represents a carbon atom and to compounds having an azapyrromethene skeleton represented by the general formula (4) in which X represents a nitrogen atom.

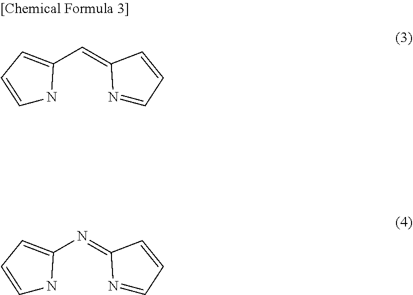

In addition, the term “pyrromethene” also refers to compounds in which a part of the pyrromethene skeleton or azapyrromethene skeleton has a condensed ring structure and a ring structure is spread.

In all the groups, hydrogen may be heavy hydrogen. The same applies to a compound described below or a partial structure thereof.

In the following description, for example, an aryl group bonded to a substituent or no substituent and having 6 to 40 carbon atoms has 6 to 40 carbon atoms including carbon atoms contained in the substituent. The same applies to other substituents that define the number of carbon atoms.

In the case of being substituted, all the above-described groups are preferably substituted with an alkyl group, a cycloalkyl group, a heterocyclic group, an alkenyl group, a cycloalkenyl group, an alkynyl group, an aryl group, a heteroaryl group, a hydroxyl group, a thiol group, an alkoxy group, an alkylthio group, an aryl ether group, an aryl thioether group, a halogen, a cyano group, an aldehyde group, an acyl group, a carboxyl group, an ester group, an amide group, an acyl group, a sulfonyl group, a sulfonic acid ester group, a sulfonamide group, an amino group, a nitro group, a silyl group, a siloxanyl group, a boryl group, a phosphine oxide group, or an oxo group, and more preferably with a specific substituent mentioned as a preferable substituent in the description of each substituent. These substituents may be further substituted with a substituent described above.

The term “bonded to no substituent” in the case of being “bonded to a substituent or no substituent” means a state of being bonded to a hydrogen atom or a heavy hydrogen atom.

The same applies to the case in which a compound described below or a partial structure thereof is “bonded to a substituent or no substituent”.

The term “alkyl group” refers to a saturated aliphatic hydrocarbon group such as a methyl group, an ethyl group, an n-propyl group, an isopropyl group, an n-butyl group, a sec-butyl group, or a tert-butyl group, and the alkyl group may have a substituent or no substituent. In the case of the alkyl group bonded to a substituent, the additional substituent is not particularly limited. Examples of the additional substituent include an alkyl group, a halogen, an aryl group, and a heteroaryl group, and the same holds true in the description below. An alkyl group substituted with a halogen is also referred to as a haloalkyl group. The number of carbon atoms in the alkyl group is not particularly limited, and is preferably in the range of 1 or more and 20 or less, and more preferably 1 or more and 8 or less from the viewpoints of easy availability and cost.

The term “cycloalkyl group” refers to a saturated alicyclic hydrocarbon group such as a cyclopropyl group, a cyclohexyl group, a norbornyl group, or an adamantyl group, and the cycloalkyl group may have a substituent or no substituent. A cycloalkyl group substituted with a halogen is also referred to as a cyclohaloalkyl group. The number of carbon atoms in the alkyl group moiety is not particularly limited, and is preferably in the range of 3 or more and 20 or less.

The term “heterocyclic group” refers to an aliphatic ring having an atom other than carbon in the ring, such as a pyran ring, a piperidine ring, or a cyclic amide, and the heterocyclic group may have a substituent or no substituent. The number of carbon atoms in the heterocyclic group is not particularly limited, and is preferably in the range of 2 or more and 20 or less.

The term “alkenyl group” refers to an unsaturated aliphatic hydrocarbon group including a double bond, such as a vinyl group, an allyl group, or a butadienyl group, and the alkenyl group may have a substituent or no substituent. The number of carbon atoms in the alkenyl group is not particularly limited, and is preferably in the range of 2 or more and 20 or less.

The term “cycloalkenyl group” refers to an unsaturated alicyclic hydrocarbon group including a double bond, such as a cyclopentenyl group, a cyclopentadienyl group, or a cyclohexenyl group, and the cycloalkenyl group may have a substituent or no substituent.

The term “alkynyl group” refers to an unsaturated aliphatic hydrocarbon group including a triple bond, such as an ethynyl group, and the alkynyl group may have a substituent or no substituent. The number of carbon atoms in the alkynyl group is not particularly limited, and is preferably in the range of 2 or more and 20 or less.

The term “aryl group” refers to an aromatic hydrocarbon group such as a phenyl group, a biphenyl group, a terphenyl group, a naphthyl group, a fluorenyl group, a benzofluorenyl group, a dibenzofluorenyl group, a phenanthryl group, an anthracenyl group, a benzophenanthryl group, a benzoanthracenyl group, a chrysenyl group, a pyrenyl group, a fluoranthenyl group, a triphenylenyl group, a benzofluoranthenyl group, a dibenzoanthracenyl group, a perylenyl group, or a helicenyl group. Among these groups, a phenyl group, a biphenyl group, a terphenyl group, a naphthyl group, a fluorenyl group, a phenanthryl group, an anthracenyl group, a pyrenyl group, a fluoranthenyl group, and a triphenylenyl group are preferable. The aryl group may have a substituent or no substituent. An aryl group substituted with a halogen is also referred to as a haloaryl group. The number of carbon atoms in the aryl group is not particularly limited, and is preferably in the range of 6 or more and 40 or less, and more preferably 6 or more and 30 or less.

In a substituted phenyl group having two adjacent carbon atoms each having a substituent, the substituents may form a ring structure. The resulting group may correspond to any one or more of a “substituted phenyl group”, an “aryl group having a structure in which two or more rings are condensed”, and a “heteroaryl group having a structure in which two or more rings are condensed” depending on the structure.

The term “heteroaryl group” refers to a cyclic aromatic group having one or a plurality of atoms other than carbon in the ring, such as a pyridyl group, a furanyl group, a thiophenyl group, a quinolinyl group, an isoquinolinyl group, a pyrazinyl group, a pyrimidyl group, a pyridazinyl group, a triazinyl group, a naphthyridinyl group, a cinnolinyl group, a phthalazinyl group, a quinoxalinyl group, a quinazolinyl group, a benzofuranyl group, a benzothiophenyl group, an indolyl group, a dibenzofuranyl group, a dibenzothiophenyl group, a carbazolyl group, a benzocarbazolyl group, a carbolinyl group, an indolocarbazolyl group, a benzofurocarbazolyl group, a benzothienocarbazolyl group, a dihydroindenocarbazolyl group, a benzoquinolinyl group, an acridinyl group, a dibenzoacridinyl group, a benzoimidazolyl group, an imidazopyridyl group, a benzoxazolyl group, a benzothiazolyl group, or a phenanthrolinyl group. The term “naphthyridinyl group” refers to any one of a 1,5-naphthyridinyl group, a 1,6-naphthyridinyl group, a 1,7-naphthyridinyl group, a 1,8-naphthyridinyl group, a 2,6-naphthyridinyl group, and a 2,7-naphthyridinyl group. The heteroaryl group may have a substituent or no substituent. The number of carbon atoms in the heteroaryl group is not particularly limited, and is preferably in the range of 2 or more and 40 or less, and more preferably 2 or more and 30 or less.

The term “alkoxy group” refers to a functional group having an aliphatic hydrocarbon group bonded via an ether bond, such as a methoxy group, an ethoxy group, or a propoxy group, and this aliphatic hydrocarbon group may have a substituent or no substituent. An alkoxy group substituted with a halogen is also referred to as a haloalkoxy group. The number of carbon atoms in the alkoxy group is not particularly limited, and is preferably in the range of 1 or more and 20 or less.

The alkylthio group is a group in which an oxygen atom of an ether bond in an alkoxy group is substituted with a sulfur atom. The hydrocarbon group in the alkylthio group may have a substituent or no substituent. The number of carbon atoms in the alkylthio group is not particularly limited, and is preferably in the range of 1 or more and 20 or less.

The term “aryl ether group” refers to a functional group having an aromatic hydrocarbon group bonded via an ether bond, such as a phenoxy group, and the aromatic hydrocarbon group may have a substituent or no substituent. An aryl ether group substituted with a halogen is also referred to as a haloaryl ether group. The number of carbon atoms in the aryl ether group is not particularly limited, and is preferably in the range of 6 or more and 40 or less.

The aryl thioether group is a group in which an oxygen atom of an ether bond in an aryl thioether group is substituted with a sulfur atom. The aromatic hydrocarbon group in the aryl thioether group may have a substituent or no substituent. The number of carbon atoms in the aryl thioether group is not particularly limited, and is preferably in the range of 6 or more and 40 or less.

The term “halogen” refers to an atom selected from fluorine, chlorine, bromine, and iodine.

The cyano group is a functional group having a structure represented by —C═N. Here, the carbon atom is bonded to another functional group.

The aldehyde group is a functional group having a structure represented by —C(═O)H. Here, the carbon atom is bonded to another functional group.

The term “acyl group” refers to a functional group having an alkyl group, a cycloalkyl group, an alkenyl group, an alkynyl group, an aryl group, or a heteroaryl group bonded via a carbonyl group, such as an acetyl group, a propionyl group, a benzoyl group, or an acrylyl group, and these substituents may be further substituted. The number of carbon atoms in the acyl group is not particularly limited, and is preferably 2 or more and 40 or less, and more preferably 2 or more and 30 or less.

The term “ester group” refers to a functional group having, for example, an alkyl group, a cycloalkyl group, an aryl group, or a heteroaryl group bonded via an ester bond, and these substituents may be further substituted. The number of carbon atoms in the ester group is not particularly limited, and is preferably in the range of 1 or more and 20 or less. More specific examples include a methyl ester group such as a methoxycarbonyl group, an ethyl ester group such as an ethoxycarbonyl group, a propyl ester group such as a propoxycarbonyl group, a butyl ester group such as a butoxycarbonyl group, an isopropyl ester group such as an isopropoxymethoxycarbonyl group, a hexyl ester group such as a hexyloxycarbonyl group, and a phenyl ester group such as a phenoxycarbonyl group.

The term “amide group” refers to a functional group having, for example, an alkyl group, a cycloalkyl group, an aryl group, or a heteroaryl group bonded via an amide bond, and these substituents may be further substituted. The number of carbon atoms in the amide group is not particularly limited, and is preferably in the range of 1 or more and 20 or less. More specific examples include a methylamide group, an ethylamide group, a propylamide group, a butyramide group, an isopropylamide group, a hexylamide group, and a phenylamide group.

The term “sulfonyl group” refers to a functional group having, for example, an alkyl group, a cycloalkyl group, an aryl group, or a heteroaryl group bonded via a bond of —S(═O)₂—, and these substituents may be further substituted. The number of carbon atoms in the sulfonyl group is not particularly limited, and is preferably in the range of 1 or more and 20 or less.

The term “sulfonic acid ester group” refers to a functional group having, for example, an alkyl group, a cycloalkyl group, an aryl group, or a heteroaryl group bonded via a sulfonic acid ester bond. Here, the sulfonic acid ester bond refers to a bond in which a carbonyl moiety in an ester bond, that is, —C(═O)— is substituted with a sulfonyl moiety, that is, —S(═O)₂—. These substituents may be further substituted. The number of carbon atoms in the sulfonic acid ester group is not particularly limited, and is preferably in the range of 1 or more and 20 or less.

The term “sulfonamide group” refers to a functional group having, for example, an alkyl group, a cycloalkyl group, an aryl group, or a heteroaryl group bonded via a sulfonamide bond. Here, the sulfonamide bond refers to a bond in which a carbonyl moiety in an ester bond, that is, —C(═O)— is substituted with a sulfonyl moiety, that is, —S(═O)₂—. These substituents may be further substituted. The number of carbon atoms in the sulfonamide group is not particularly limited, and is preferably in the range of 1 or more and 20 or less.

The amino group is an amino group bonded to a substituent or no substituent. Examples of the substituent in the case of an amino group bonded to a substituent include an aryl group, a heteroaryl group, a linear alkyl group, and a branched alkyl group. As the aryl group and the heteroaryl group, a phenyl group, a naphthyl group, a pyridyl group, and a quinolinyl group are preferable. These substituents may be further substituted. The number of carbon atoms is not particularly limited, and is preferably in the range of 2 or more and 50 or less, more preferably 6 or more and 40 or less, and particularly preferably 6 or more and 30 or less.

The term “silyl group” refers to a functional group having a bonding silicon atom bonded to a substituent or no substituent, such as an alkylsilyl group such as a trimethylsilyl group, a triethylsilyl group, a tert-butyldimethylsilyl group, a propyldimethylsilyl group, or a vinyldimethylsilyl group, or an arylsilyl group such as a phenyldimethylsilyl group, a tert-butyldiphenylsilyl group, a triphenylsilyl group, or a trinaphthylsilyl group. The substituent on the silicon may be further substituted. The number of carbon atoms in the silyl group is not particularly limited, and is preferably in the range of 1 or more and 30 or less.

The term “siloxanyl group” refers to a silicon compound group that is bonded via an ether bond, such as a trimethylsiloxanyl group. The substituent on the silicon may be further substituted.

The boryl group is a boryl group bonded to a substituent or no substituent. Examples of the substituent in the case of a boryl group bonded to a substituent include an aryl group, a heteroaryl group, a linear alkyl group, a branched alkyl group, an aryl ether group, an alkoxy group, and a hydroxyl group, and among the groups, an aryl group and an aryl ether group are preferable.

The phosphine oxide group is a group represented by —P(═O)R⁶⁰R⁶¹. R⁶⁰ and R⁶¹ may be the same or different from each other, and are each selected from a halogen atom, an alkyl group, a cycloalkyl group, a heterocyclic group, an aryl group, a heteroaryl group, a hydroxyl group, a thiol group, an alkoxy group, an alkylthio group, an aryl ether group, an aryl thioether group, a halogen, a cyano group, an acyl group, an ester group, an amide group, and a ring structure with an adjacent group.

The oxo group is a functional group in which an oxygen atom is bonded to a carbon atom through a double bond, that is, a structure of ═O.

The compound represented by the general formula (1) or (2) is a complex in which a pyrromethene compound is coordinated to a metal having a valence of m, M. The valence m of the metal is not particularly limited as long as each metal can take the valence, and from the viewpoint of forming a stable coordination state, the value of m is preferably 2 to 4, and more preferably 3. The metal M is selected from the above, and M is preferably boron from the viewpoints of light emission characteristics such as chromaticity and luminous efficiency, thermal stability in sublimation purification and deposition, durability of the element, and ease of synthesis.

L represents a ligand other than pyrromethene with respect to the metal M. L is selected from the above, and is preferably an alkoxy group, an aryl ether group, a halogen, or a cyano group from the viewpoints of a light emission characteristic and thermal stability. Furthermore, from the viewpoints of obtaining a further high fluorescence quantum yield in a stable excited state and improving the durability, L is more preferably a fluorine atom, a fluorine-containing alkyl group, a fluorine-containing alkoxy group, a fluorine-containing aryl group, or a cyano group, still more preferably a fluorine atom or a cyano group, and most preferably a fluorine atom. These groups are an electron withdrawing group, and can reduce the electron density of the pyrromethene skeleton and increase the stability of the compound.

In the case that m is 3 or more, that is, in the case that two or more L is bonded to M, L may be the same or different from each other, and are preferably the same from the viewpoint of ease of synthesis.

The pyrromethene metal complex exhibits a high fluorescence quantum yield because of its strong and highly planar skeleton. Furthermore, the pyrromethene metal complex can achieve efficient light emission and high color purity because of its small peak half-value width in the emission spectrum.

Examples of the method of making such a pyrromethene metal complex to emit red light include a method in which an aromatic hydrocarbon ring or an aromatic heterocyclic ring is directly bonded to a pyrromethene metal complex skeleton to extend the conjugation and lengthen the emission wavelength. However, if the rings are merely bonded to the pyrromethene metal complex skeleton, the structure changes to a plurality of stable structures in the excited state (hereinafter, this change is referred to as “structural relaxation”), and the change leads to light emission from various energy states, resulting in deactivation. In this case, there has been a problem that the broad emission spectrum leads to a large half-value width, resulting in deterioration of the color purity. As described above, in the case of obtaining a red-light-emitting material with a pyrromethene metal complex, molecular design is to be devised in order to improve the characteristic.

Therefore, in embodiments of the present invention, as represented by the general formula (1) or (2), a bridging structure Y¹ is introduced between the pyrrole ring of the pyrromethene skeleton and Ar¹. Ar¹ is the aromatic hydrocarbon ring or the aromatic heterocyclic ring described above, and is directly bonded to the pyrromethene metal complex skeleton. The double bond shown as a part of Ar¹ in the general formula (1) or (2) represents a part of the aromatic ring, and indicates that the carbon atom directly bonded to the pyrromethene skeleton and the carbon atom to which the bridging structure Y¹ is bonded are adjacent to each other.

The introduction of the bridging structure limits the rotation and the vibration of the aromatic hydrocarbon ring or the aromatic heterocyclic ring, and as a result, excessive structural relaxation of the pyrromethene metal complex can be suppressed in the excited state to obtain a sharp emission spectrum (and reduce the half-value width of the emission spectrum). If this is used in an emissive material, light emission with good color purity can be obtained.

However, in the case that the bridging structure includes one atom or two atoms in series, the planarity is too high in the pyrromethene metal complex skeleton and the aromatic hydrocarbon ring or the aromatic heterocyclic ring, and as a result, conjugation is extended to lengthen the light emission peak wavelength excessively, and the target chromaticity is difficult to achieve. In order to achieve both narrowing of the emission spectrum and adjustment of the light emission peak wavelength, it is preferable to fix the pyrromethene metal complex skeleton and the aromatic hydrocarbon ring or the aromatic heterocyclic ring in a slightly twisted state. For this reason, Y¹ is a bridging structure in which three or more atoms are bonded in series. Meanwhile, if the bridging structure is too long, the limitation of the rotation and the vibration in the molecule is so loose that structural relaxation easily occurs, and as a result, the color purity deteriorates. Furthermore, the structure has a large distortion, and therefore synthesis is difficult. From this viewpoint, the number of atoms bonded in series is preferably 5 or less, and Y¹ is preferably a bridging structure in which three atoms are bonded in series.

The atom included in Y¹ is as described above, and is preferably selected from a carbon atom bonded to a substituent or no substituent, an oxygen atom, and a sulfur atom among the above-described atoms, and is more preferably a carbon atom bonded to a substituent or no substituent from the viewpoints of thermal stability and ease of synthesis.

Furthermore, from the viewpoint of a light emission characteristic, Y¹ preferably has a structure represented by the general formula (5A) or (5B).

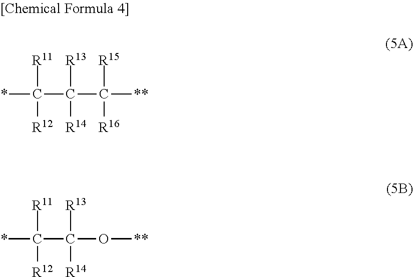

In the formula, * represents a linking moiety with a pyrrole ring, ** represents a linking moiety with Ar¹, R¹¹ to R¹⁶ are the same or different from each other, and are each selected from the functional groups from which R¹ to R⁵ in the general formula (1) or (2) are selected and an oxo group. In particular, from the viewpoint of thermal stability or ease of synthesis, R¹¹ to R¹⁶ are preferably selected from a hydrogen atom, an alkyl group, and an oxo group.

Z¹ in the general formula (2) is a bridging structure linking the other pyrrole ring, that is, the pyrrole ring that is not the pyrrole ring to which Y¹ is linking, with Ar² in the pyrromethene skeleton. Ar² is the aromatic hydrocarbon ring or the aromatic heterocyclic ring described above, and is directly bonded to the pyrromethene metal complex skeleton. The double bond shown as a part of Ar² in the general formula (2) represents a part of the aromatic ring, and indicates that the carbon atom directly bonded to the pyrromethene skeleton and the carbon atom to which the bridging structure Z¹ is bonded are adjacent to each other.

Z¹ is a bridging structure in which one or more atoms are bonded, and it is preferable that one to three atoms be bonded in series from the viewpoints of color purity and ease of synthesis.

The atom included in Z¹ is as described above, and is preferably selected from a carbon atom bonded to a substituent or no substituent, an oxygen atom, and a sulfur atom among the above-described atoms, and is more preferably a carbon atom bonded to a substituent or no substituent from the viewpoints of thermal stability and ease of synthesis.

X is selected from C—R⁵ and N as described above. Here, X is preferably C—R⁵ from the viewpoint of ease of control to the appropriate chromaticity for red-light emission in use of the emissive material according to embodiments of the present invention as a display device or an illumination device.

R⁵ is selected from the above-described functional groups, and from the viewpoint of electrical stability or thermal stability, R⁵ is preferably a hydrogen atom, an alkyl group bonded to a substituent or no substituent, an aryl group bonded to a substituent or no substituent, or a heteroaryl group bonded to a substituent or no substituent, and more preferably an aryl group bonded to a substituent or no substituent, or a heteroaryl group bonded to a substituent or no substituent. Specific examples of the functional group as R⁵ include a phenyl group bonded to a substituent or no substituent, a naphthyl group bonded to a substituent or no substituent, a phenanthryl group bonded to a substituent or no substituent, an anthryl group bonded to a substituent or no substituent, and a dibenzofuranyl group bonded to a substituent or no substituent, and a phenyl group bonded to a substituent or no substituent and a naphthyl group bonded to a substituent or no substituent are more preferable.

Furthermore, in order to improve the luminous efficiency, it is effective to suppress the rotation and the vibration of the substituent at the bridge-head position of the pyrromethene boron complex to reduce the energy loss for improvement in the fluorescence quantum yield. From this viewpoint, R⁵ is preferably a group represented by the general formula (6).

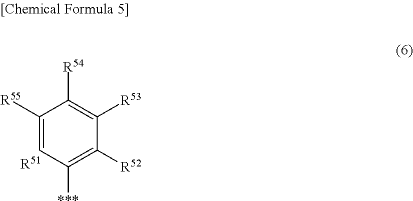

In the formula, *** represents a bonding moiety with a carbon atom. R⁵¹ and R⁵² are the same or different from each other, and are each selected from the group consisting of an alkyl group bonded to a substituent or no substituent, an aryl group bonded to a substituent or no substituent, and a heteroaryl group bonded to a substituent or no substituent. From the viewpoint of ease of production, R⁵¹ and R⁵² are each preferably an alkyl group bonded to a substituent or no substituent, and more preferably a methyl group. Meanwhile, it is preferable that at least one of R⁵¹ or R⁵² be an aryl group bonded to a substituent or no substituent or a heteroaryl group bonded to a substituent or no substituent because such a state yields a further large effect of suppressing the rotation and is advantageous for improving the fluorescence quantum yield. R⁵³ to R⁵⁵ are the same or different from each other, and are each selected from a hydrogen atom, an alkyl group, a cycloalkyl group, a heterocyclic group, an alkenyl group, a cycloalkenyl group, an alkynyl group, an aryl group, a heteroaryl group, a hydroxyl group, a thiol group, an alkoxy group, an alkylthio group, an aryl ether group, an aryl thioether group, a halogen, a cyano group, an aldehyde group, an acyl group, a carboxyl group, an ester group, an amide group, a sulfonyl group, a sulfonic acid ester group, a sulfonamide group, an amino group, a nitro group, a silyl group, and a ring structure with an adjacent group, and the functional groups that are substituted. R⁵⁴ particularly affects the light emission peak wavelength, and R⁵⁴ as an electron-donating group shifts the light emission peak wavelength to the short wavelength side, and R⁵⁴ as an electron withdrawing group shifts the light emission peak wavelength to the long wavelength side. Specifically, examples of the electron-donating group include a methyl group, an ethyl group, a tert-butyl group, a cyclohexyl group, a methoxy group, an ethoxy group, a phenyl group, a tolyl group, a naphthyl group, a furanyl group, and a dibenzofuranyl group, and examples of the electron withdrawing group include a fluorine atom, a trifluoromethyl group, a cyano group, a pyridyl group, and a pyrimidyl group, but examples are not limited thereto.

R¹ in the general formulae (1) and (2) is a substituent that contributes to the stability and the luminous efficiency of the pyrromethene metal complex compound. Here, the term “stability” refers to the electrical stability and the thermal stability. The electrical stability means that alteration of the compound, such as decomposition, is not caused in a state where the element is continuously energized, and the thermal stability means that alteration of the compound is not caused by a heating step such as sublimation purification or deposition or by an environmental temperature around the element. Alteration of the compound leads to reduction in the luminous efficiency, and therefore the stability of the compound is important for improving the durability of the light-emitting element. In the case that Y¹ is trimethylene and R¹ is a hydrogen atom or a halogen, the stability and the luminous efficiency of the compound are greatly reduced, and therefore a pyrromethene metal complex in such a case is not the pyrromethene metal complex according to embodiments of the present invention.

R¹ is selected from the above-described functional groups, and from the viewpoint of the stability of the compound, R¹ is preferably an alkyl group bonded to a substituent or no substituent, an aryl group bonded to a substituent or no substituent, or a heteroaryl group bonded to a substituent or no substituent. From the viewpoints of the stability and the luminous efficiency of the compound, R¹ is more preferably an aryl group bonded to a substituent or no substituent. Specific examples of R¹ include a phenyl group bonded to a substituent or no substituent and a naphthyl group bonded to a substituent or no substituent.

From the viewpoint of preventing flocculation of the pyrromethene metal complexes to avoid concentration quenching, R¹ preferably has an alkyl group or an aryl group as a substituent. Specific examples of the substituent include a methyl group, an ethyl group, an isopropyl group, a tert-butyl group, and a phenyl group.

For the same reason as in the case of R¹, R² in the general formulae (1) and (2) is preferably an alkyl group bonded to a substituent or no substituent, an aryl group bonded to a substituent or no substituent, or a heteroaryl group bonded to a substituent or no substituent, and more preferably an aryl group bonded to a substituent or no substituent. Specific examples of R² include a phenyl group bonded to a substituent or no substituent and a naphthyl group bonded to a substituent or no substituent. R² preferably has an alkyl group or an aryl group as a substituent. Specific examples of the substituent include a methyl group, an ethyl group, an isopropyl group, a tert-butyl group, and a phenyl group.

R³ in the general formula (1) is preferably a hydrogen atom, an alkyl group bonded to a substituent or no substituent, or an aryl group bonded to a substituent or no substituent from the viewpoint of an optical characteristic such as chromaticity or ease of synthesis.

R⁴ in the general formula (1) is preferably an aryl group bonded to a substituent or no substituent or a heteroaryl group bonded to a substituent or no substituent from the viewpoint of the optical characteristic such as chromaticity.

From the viewpoints of lengthening the emission spectrum wavelength and obtaining red-light emission with further high color purity, another preferable example is formation of a ring structure between R² and R³ or between R³ and R⁴ in the general formula (1). However, in order to prevent excessive lengthening of the emission spectrum wavelength, the ring structure formed between R³ and R⁴ is a monocyclic ring. In particular, it is more preferable that the ring structure and pyrrole form a fused aromatic ring. Specific examples of the fused aromatic ring include, but are not limited to, an indole ring, an isoindole ring, a pyrrolopyrrole ring, a furopyrrole ring, and a thienopyrrole ring.

The molecular weight of the pyrromethene metal complex represented by the general formula (1) or (2) is not particularly limited. In the case of using the pyrromethene metal complex as a light-emitting element material, the molecular weight is preferably within a range in which the deposition step is facilitated. Specifically, from the viewpoint of obtaining a stable deposition rate, the molecular weight of the pyrromethene metal complex represented by the general formula (1) or (2) is preferably 500 or more, more preferably 600 or more, and still more preferably 700 or more. From the viewpoint of preventing decomposition due to an excessively high deposition temperature, the molecular weight is preferably 1,200 or less, and more preferably 1,000 or less.

From the viewpoint of obtaining a further sharp emission spectrum to further improve the color purity and the luminous efficiency, the pyrromethene metal complex of the present invention is preferably represented by the general formula (2).

The pyrromethene metal complex of the present invention is preferably, for example, a compound represented by any one of the following general formulae (7A) to (7M).

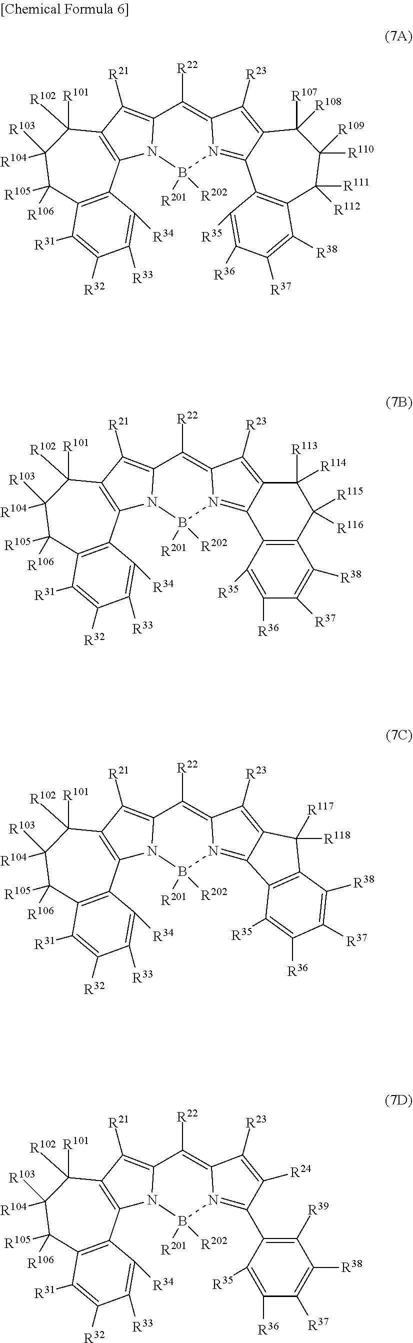

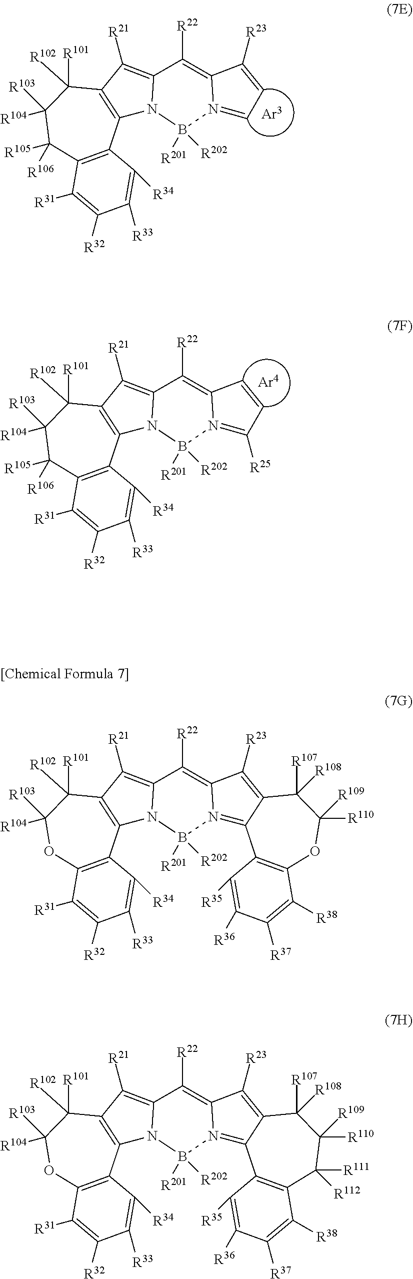

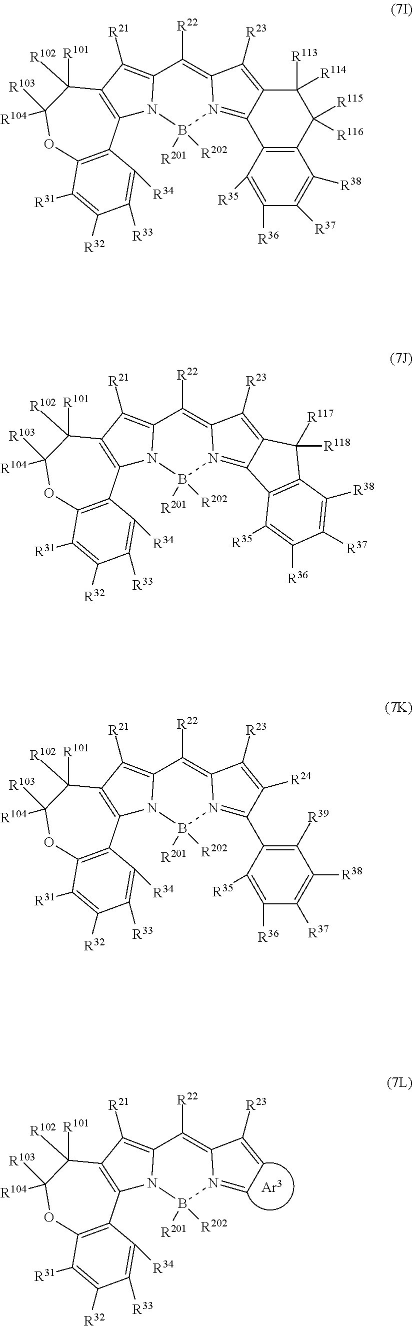

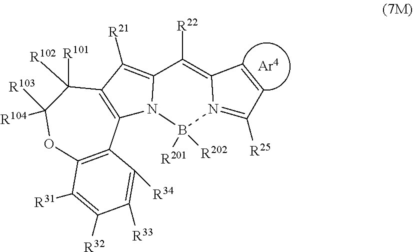

In the formulae, R²¹ to R²⁵ are the same or different from each other, and are each selected from a hydrogen atom, an alkyl group, a cycloalkyl group, a heterocyclic group, an aryl group, and a heteroaryl group, and the functional groups that are substituted. However, R²¹ is not a hydrogen atom in a case that all of R¹⁰¹ to R¹⁰⁶ are a hydrogen atom.

Among the functional groups, R²¹ and R²³ are preferably an alkyl group bonded to a substituent or no substituent, an aryl group bonded to a substituent or no substituent, or a heteroaryl group bonded to a substituent or no substituent, and more preferably an aryl group bonded to a substituent or no substituent from the viewpoint of electrical stability or thermal stability. R²² is preferably a hydrogen atom, an alkyl group bonded to a substituent or no substituent, an aryl group bonded to a substituent or no substituent, or a heteroaryl group bonded to a substituent or no substituent, and more preferably an aryl group bonded to a substituent or no substituent, or a heteroaryl group bonded to a substituent or no substituent from the viewpoint of electrical stability or thermal stability. R²⁴ and R²⁵ are preferably a hydrogen atom, an alkyl group bonded to a substituent or no substituent, or an aryl group bonded to a substituent or no substituent from the viewpoint of an optical characteristic such as chromaticity or ease of synthesis.

R³¹ to R³⁹ are the same or different from each other, and are each selected from a hydrogen atom, an alkyl group, a cycloalkyl group, a heterocyclic group, an alkenyl group, a cycloalkenyl group, an alkynyl group, an aryl group, a heteroaryl group, a hydroxyl group, a thiol group, an alkoxy group, an alkylthio group, an aryl ether group, an aryl thioether group, a halogen, a cyano group, an aldehyde group, an acyl group, a carboxyl group, an ester group, an amide group, a sulfonyl group, a sulfonic acid ester group, a sulfonamide group, an amino group, a nitro group, a silyl group, a siloxanyl group, a boryl group, a phosphine oxide group, and a ring structure with an adjacent group, and the functional groups that are substituted. These functional groups are preferably a hydrogen atom, an alkyl group, an aryl group, a heteroaryl group, or an alkoxy group from the viewpoints of a deposition characteristic and luminous efficiency.

R¹⁰¹ to R¹¹⁸ are the same or different from each other, and are each selected from a hydrogen atom, an alkyl group, a cycloalkyl group, a heterocyclic group, an alkenyl group, a cycloalkenyl group, an alkynyl group, an aryl group, a heteroaryl group, a hydroxyl group, a thiol group, an alkoxy group, an alkylthio group, an aryl ether group, an aryl thioether group, a halogen, a cyano group, an aldehyde group, an acyl group, a carboxyl group, an ester group, an amide group, a sulfonyl group, a sulfonic acid ester group, a sulfonamide group, an amino group, a nitro group, a silyl group, a siloxanyl group, a boryl group, a phosphine oxide group, and an oxo group, and the functional groups that are substituted. A ring structure is formed between any two substituents selected from R¹⁰¹ to R¹⁰⁶, between any two substituents selected from R¹⁶⁷ to R¹¹², between any two substituents selected from R¹¹³ to R¹¹⁶, or between R¹¹⁷ and R¹¹⁸, or no ring structure is formed between any two substituents selected from R¹⁰¹ to R¹⁰⁶, between any two substituents selected from R¹⁶⁷ to R¹¹², between any two substituents selected from R¹¹³ to R¹¹⁶, and between R¹¹⁷ and R¹¹⁸. Among them, it is preferable to be selected from a hydrogen atom, an alkyl group, and an oxo group from the viewpoint of thermal stability and ease of synthesis.

R²⁰¹ and R²⁰² are the same or different from each other, and are each selected from an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, and a cyano group, and the functional groups that are substituted.

Among these functional groups, an alkoxy group, an aryl ether group, a halogen, and a cyano group are preferable from the viewpoints of a light emission characteristic and thermal stability. Furthermore, from the viewpoints of obtaining a further high fluorescence quantum yield in a stable excited state and improving the durability, a fluorine atom, a fluorine-containing alkyl group, a fluorine-containing alkoxy group, a fluorine-containing aryl group, and a cyano group are more preferable, a fluorine atom and a cyano group are still more preferable, and a fluorine atom is the most preferable.

Ar³ and Ar⁴ are the same or different from each other, and are each selected from an aromatic hydrocarbon ring bonded to a substituent or no substituent and an aromatic heterocyclic ring bonded to a substituent or no substituent.

Examples of the compound represented by the general formula (1) or (2) are shown below, but are not limited thereto.

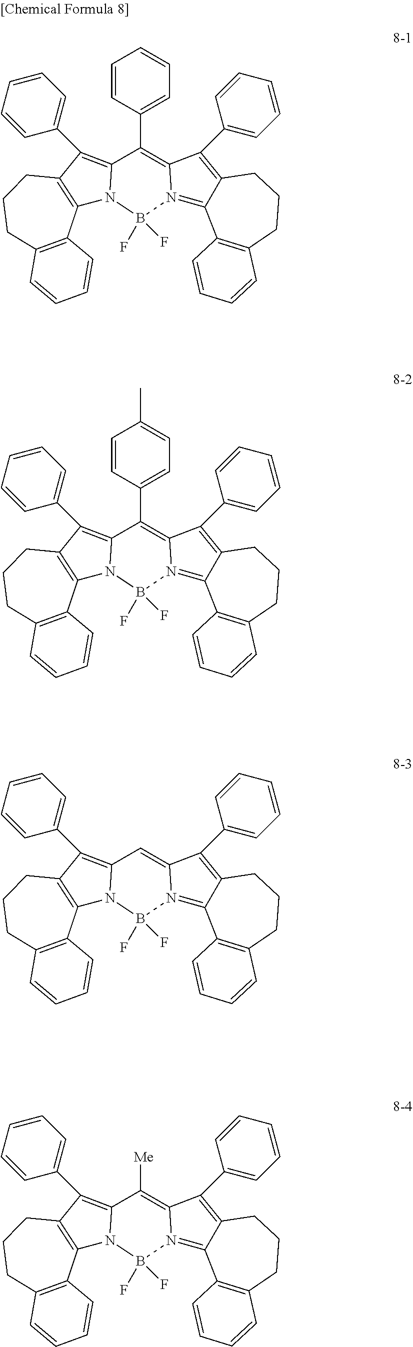

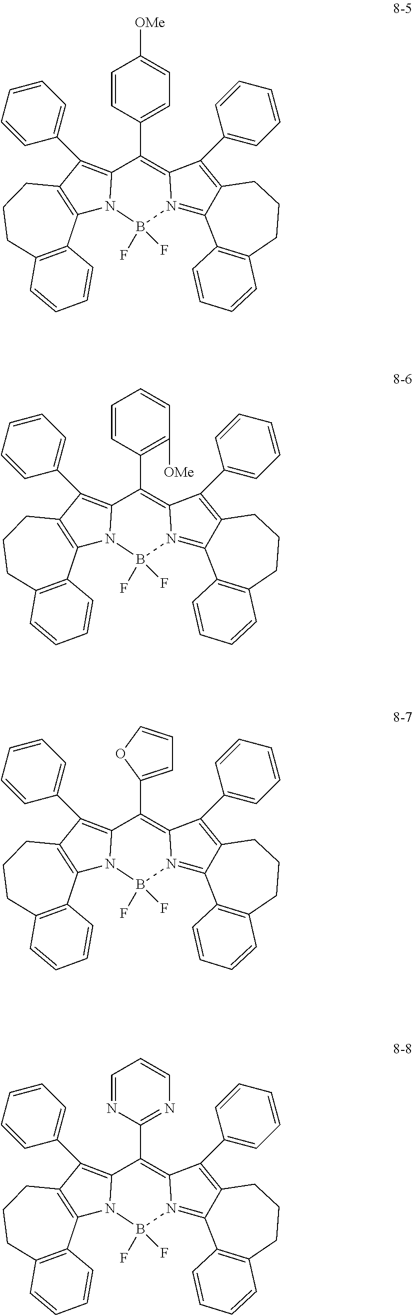

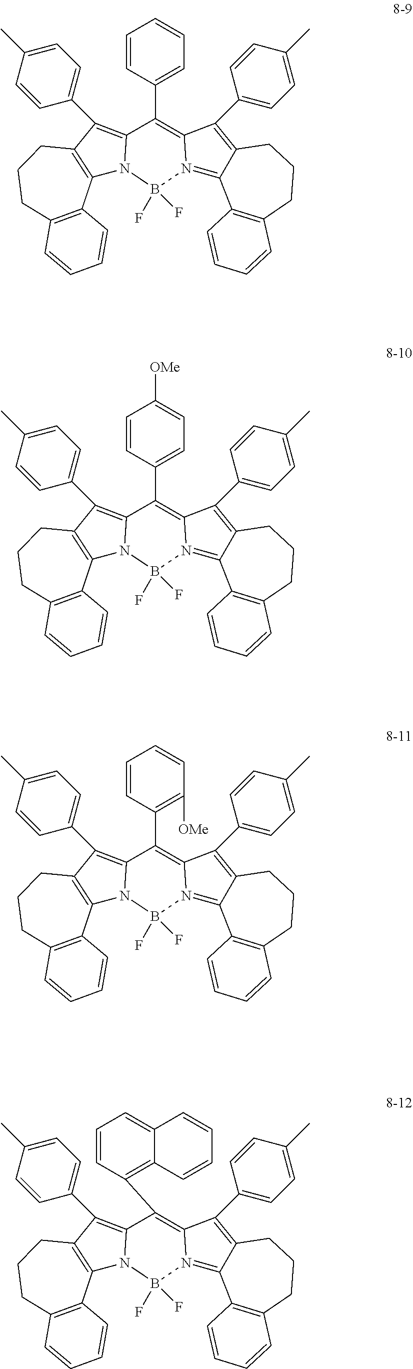

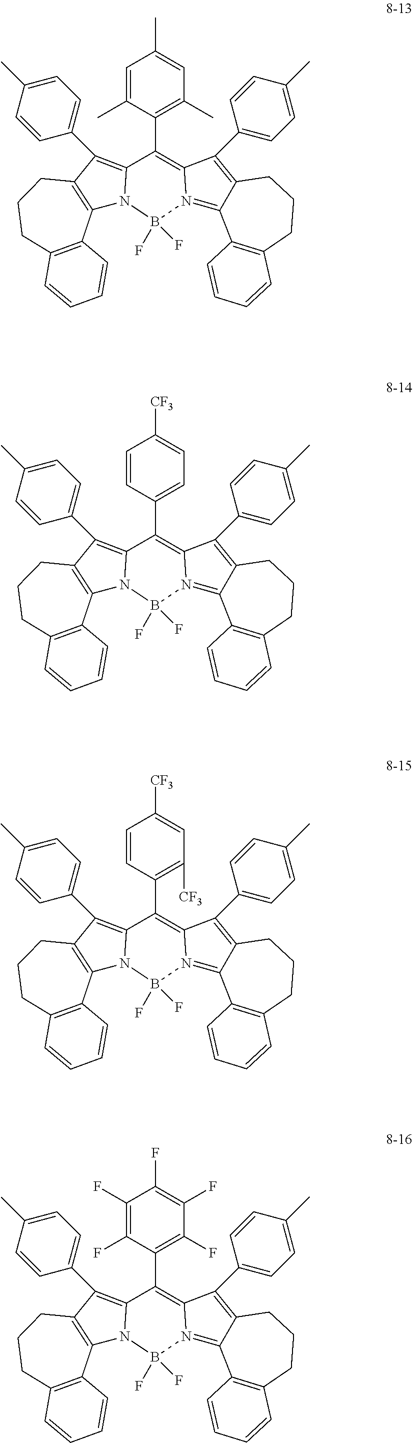

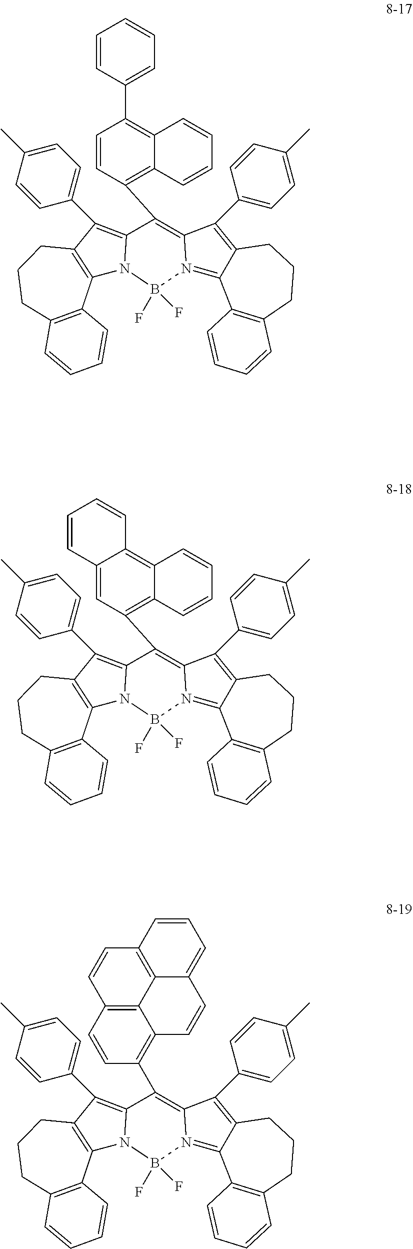

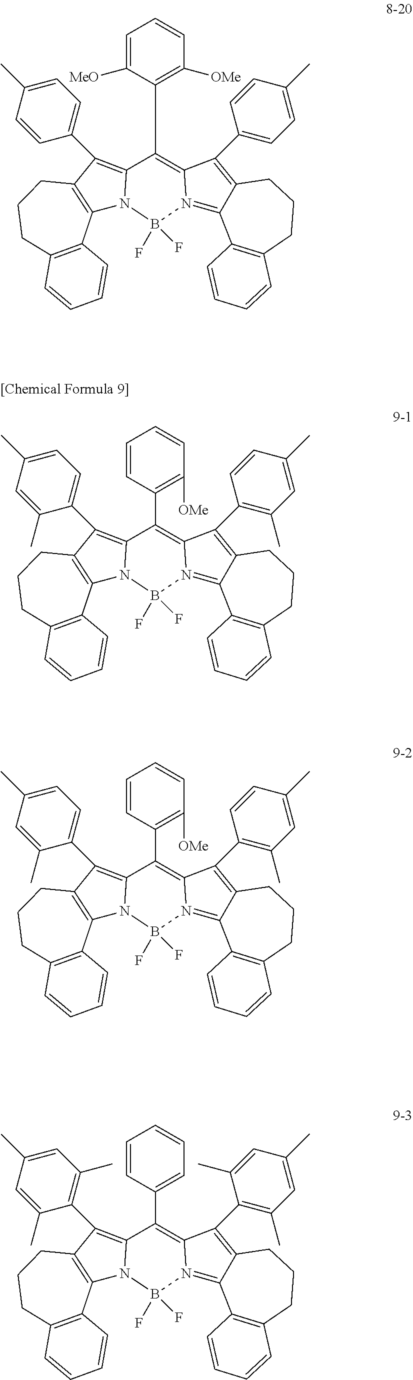

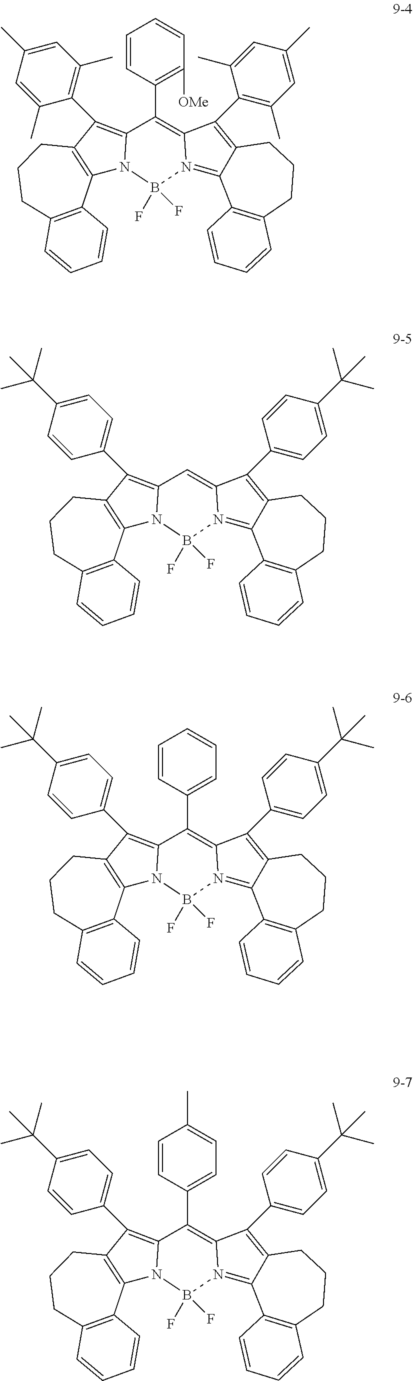

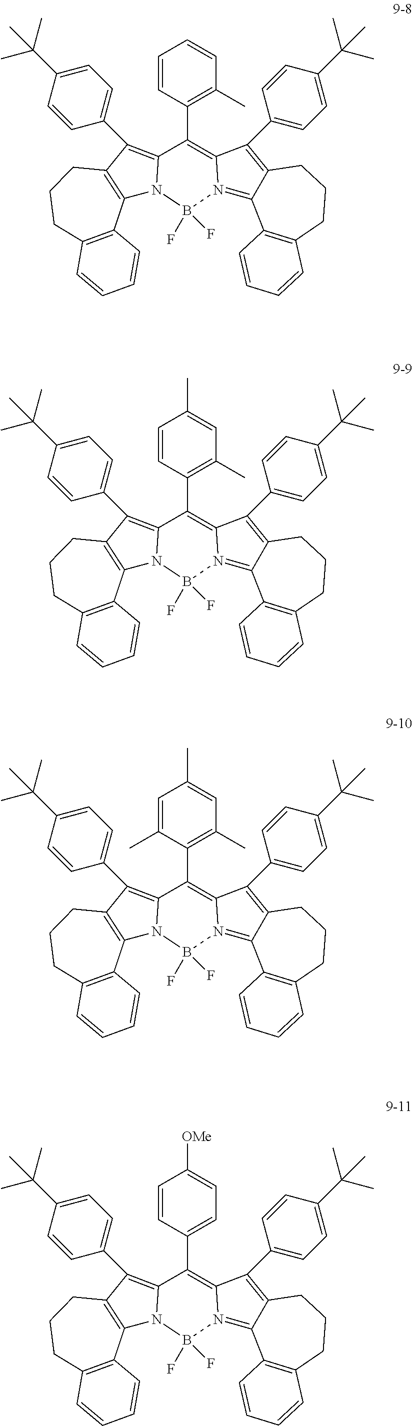

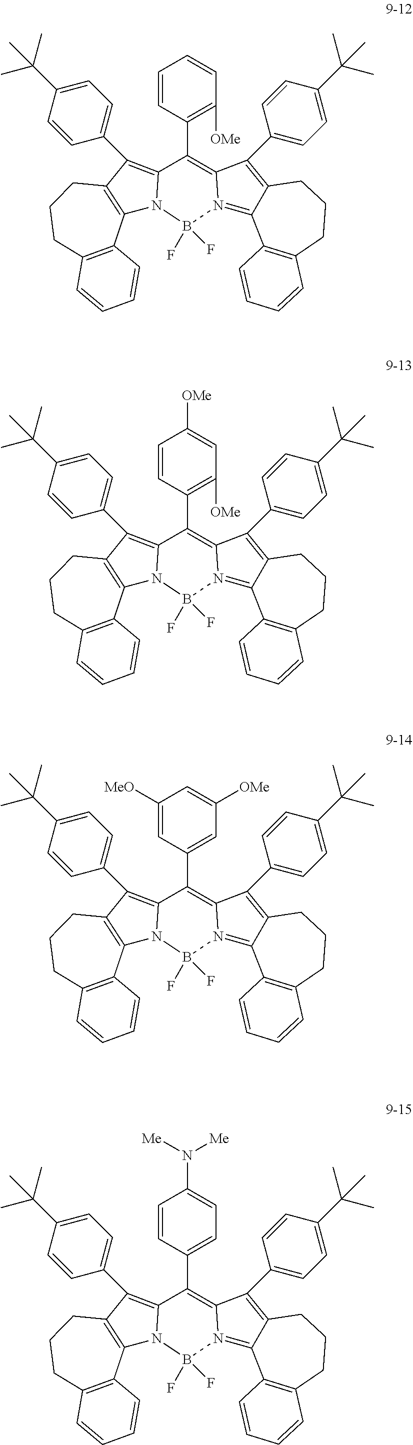

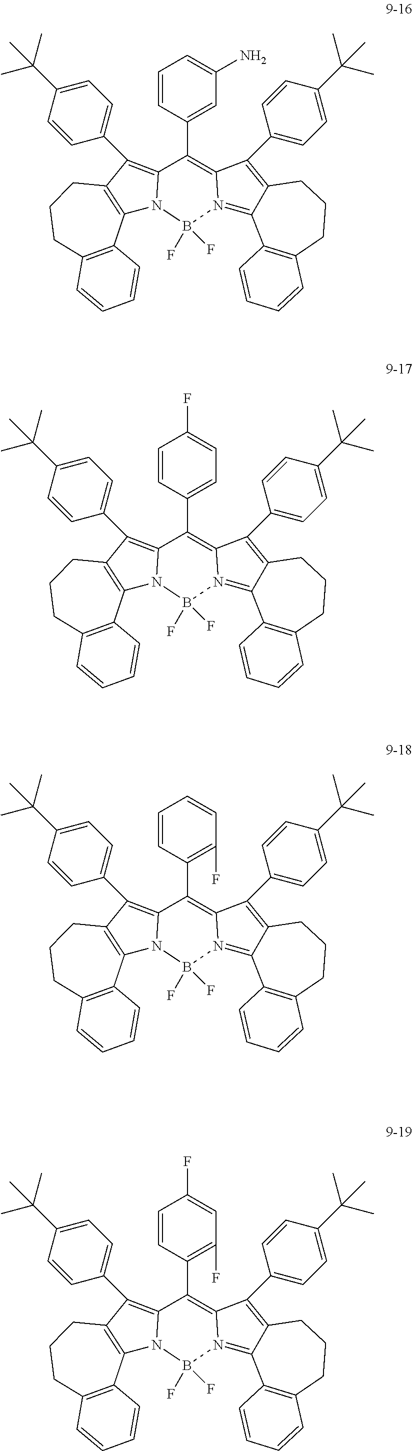

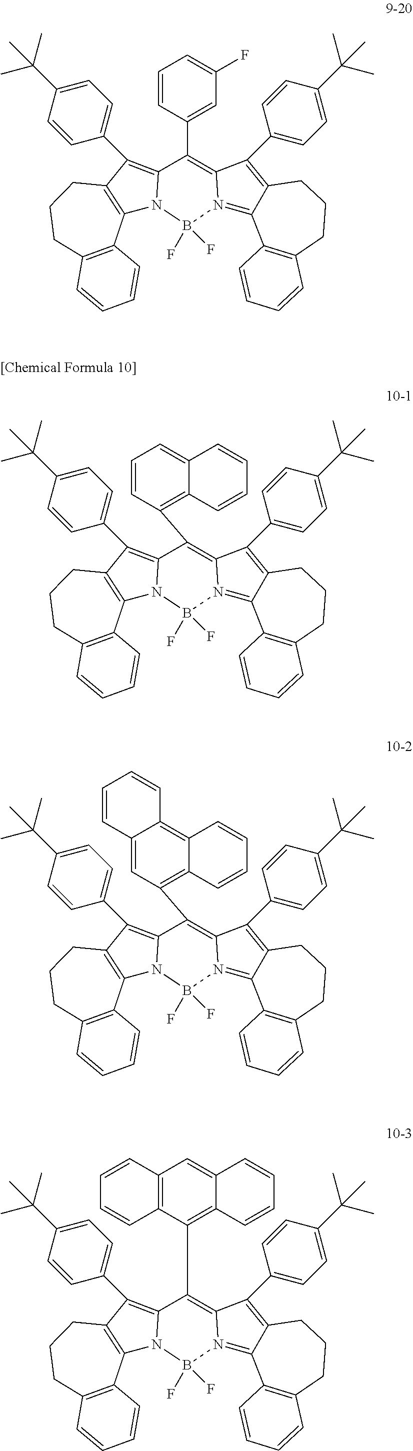

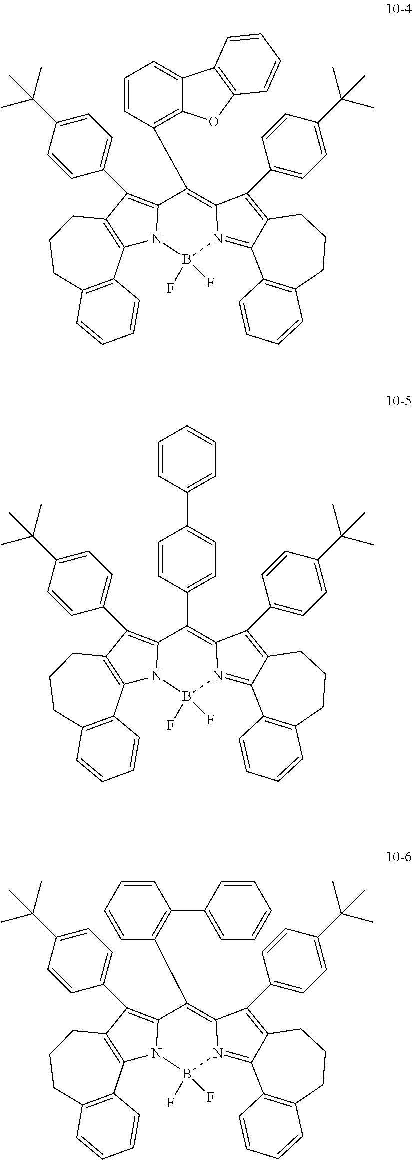

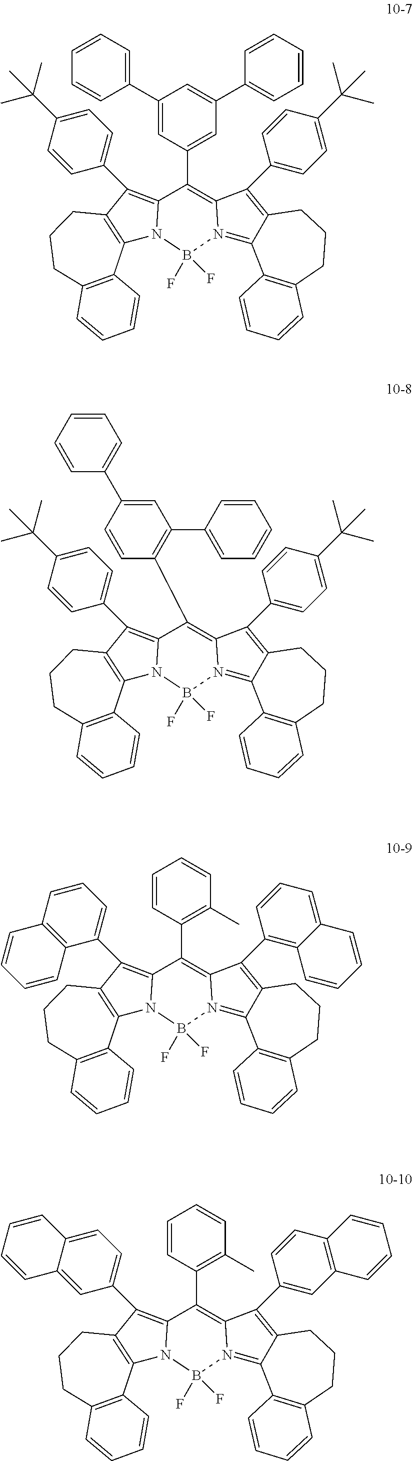

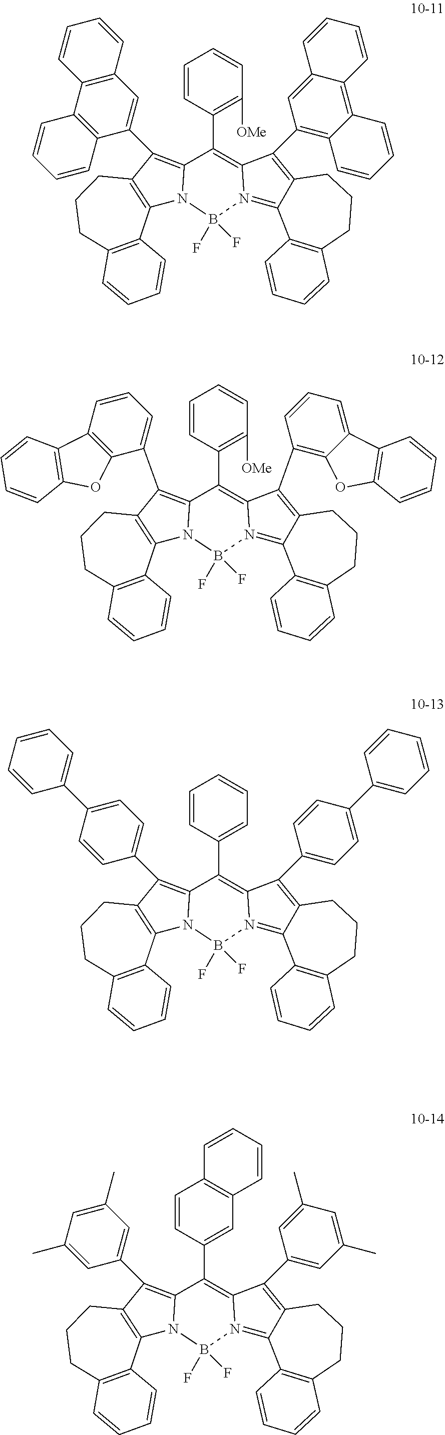

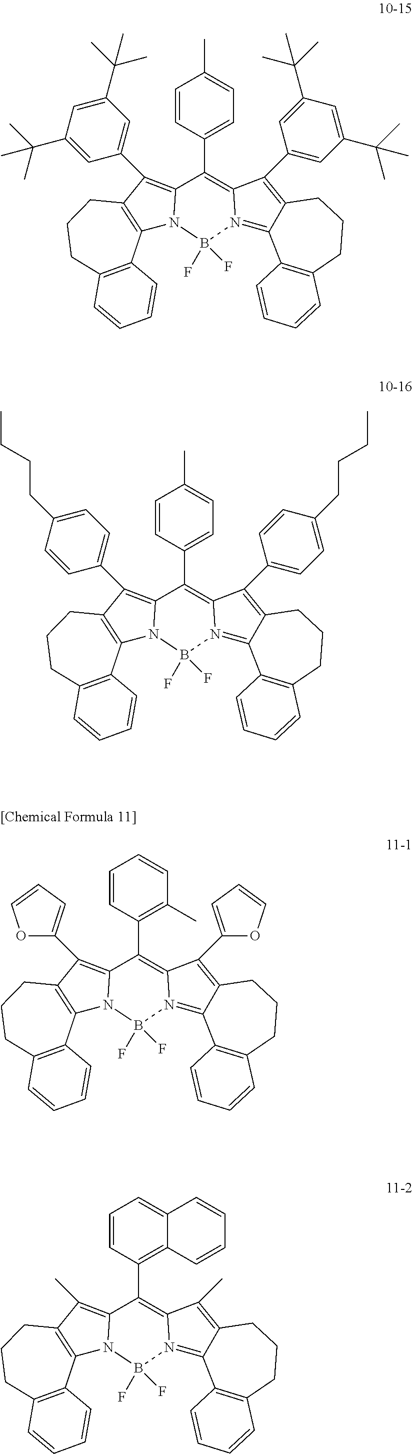

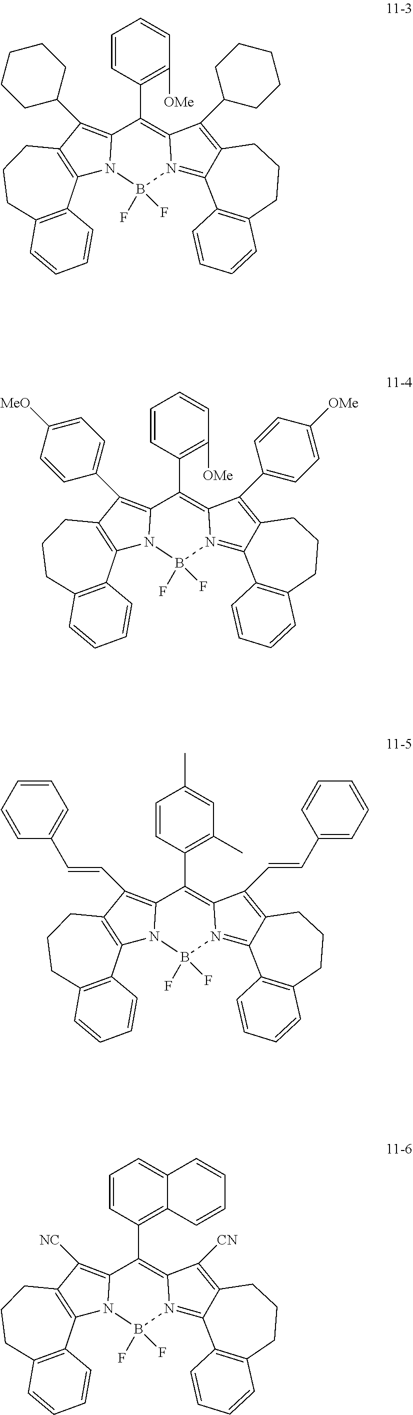

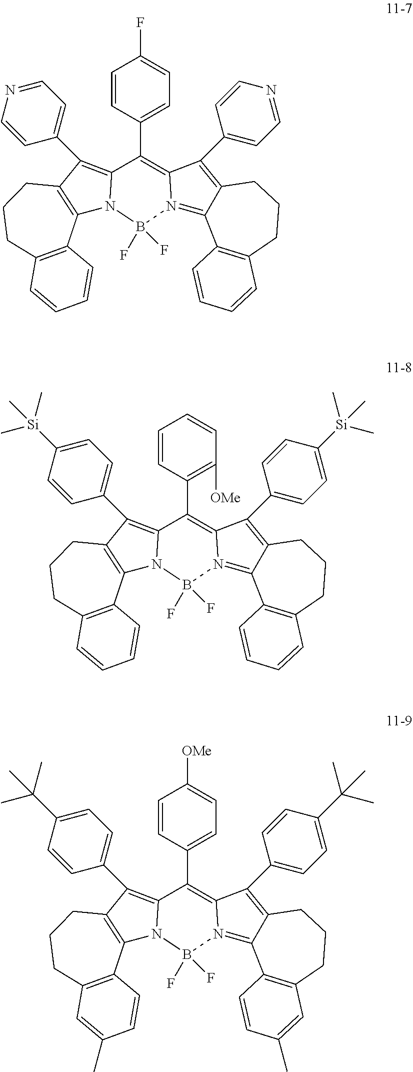

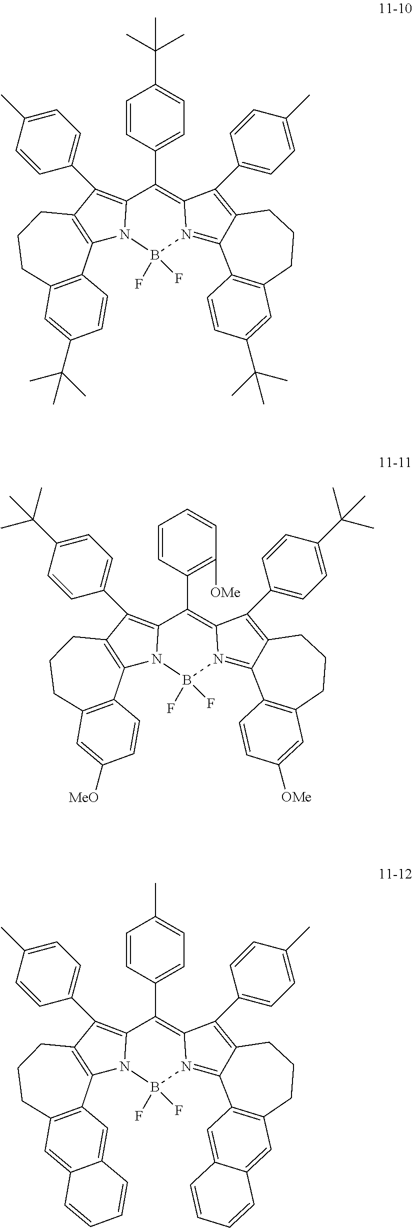

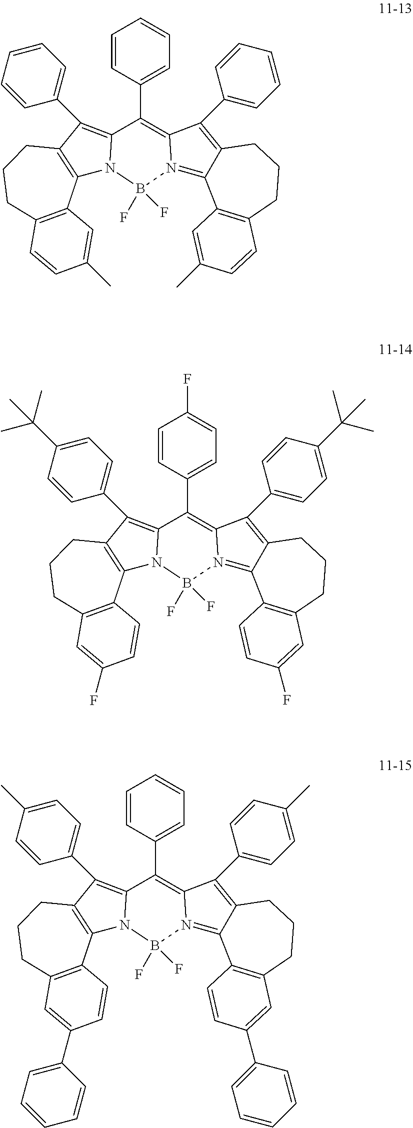

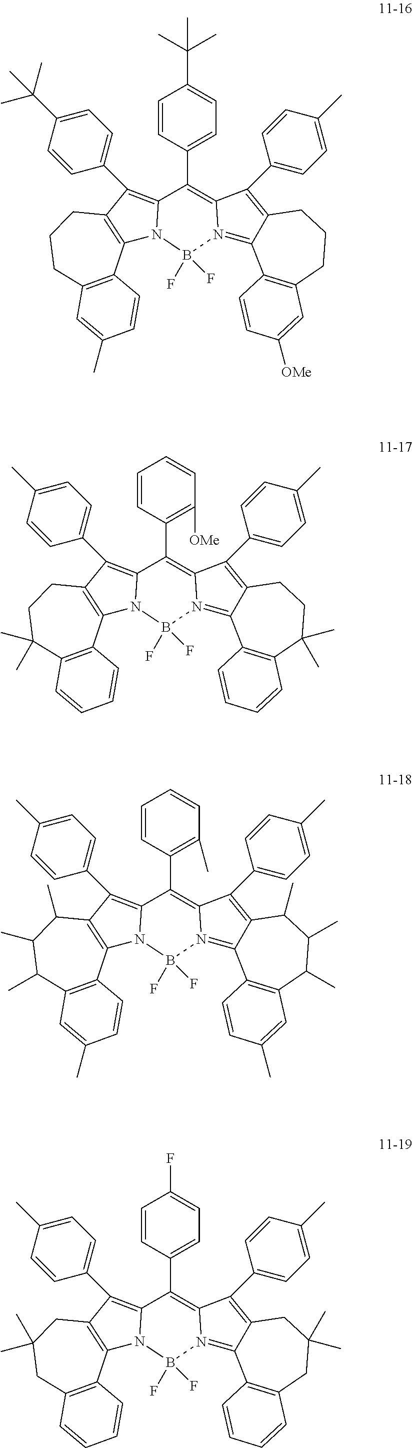

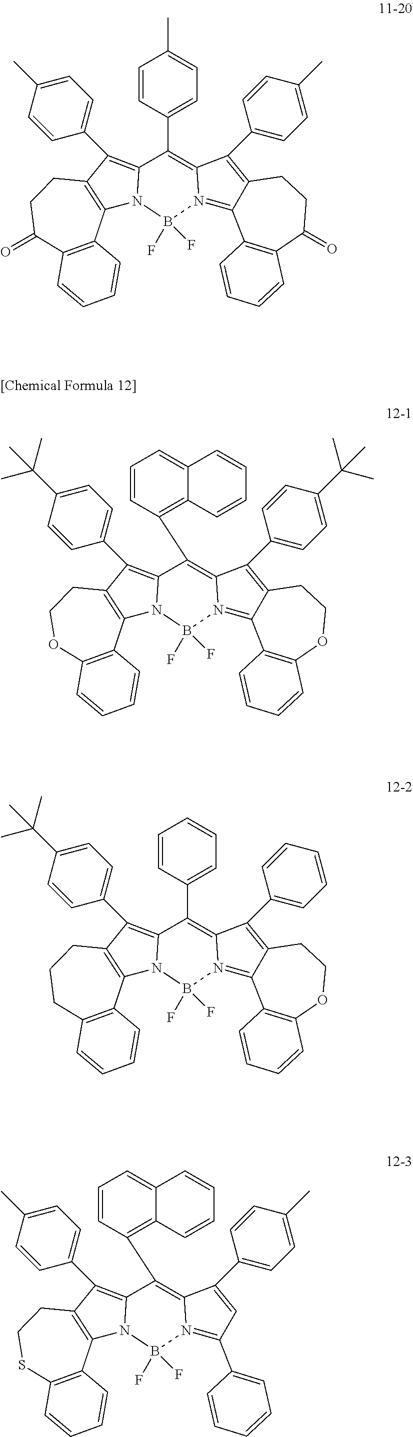

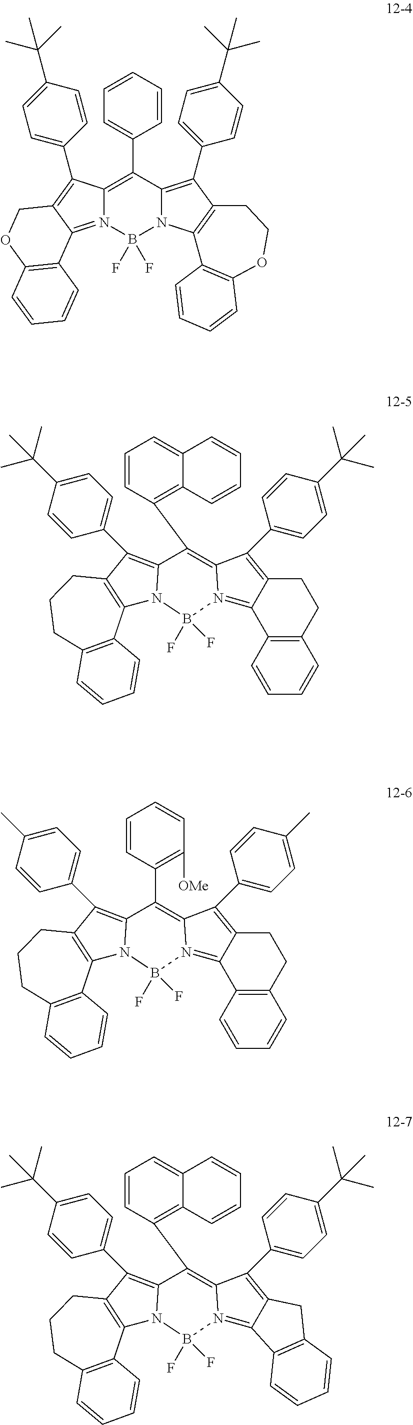

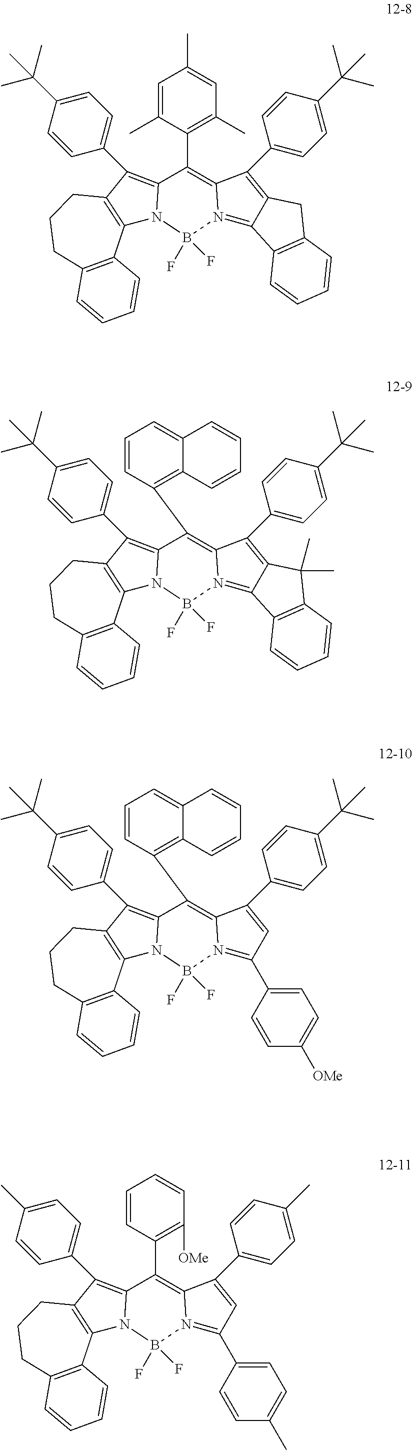

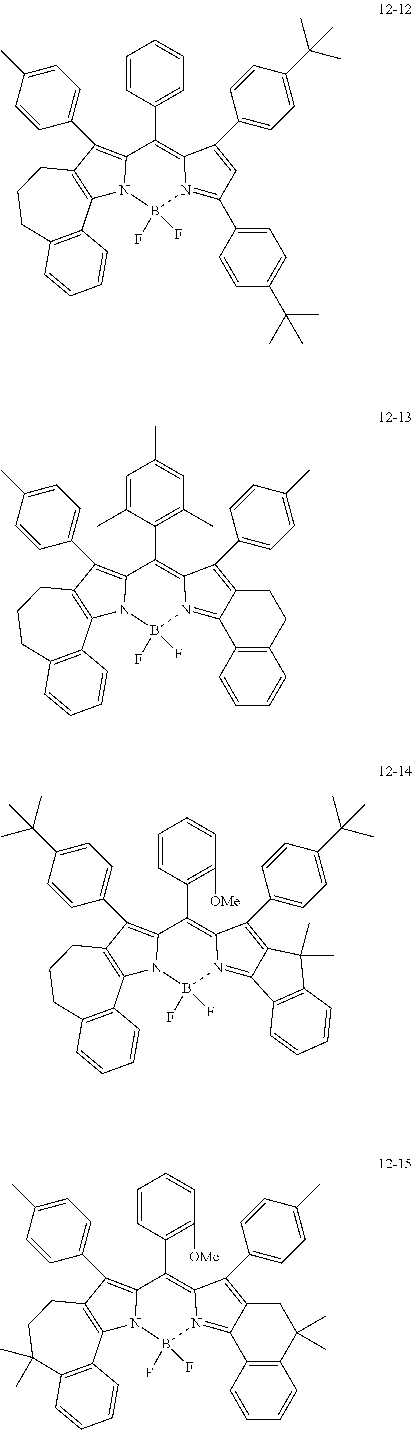

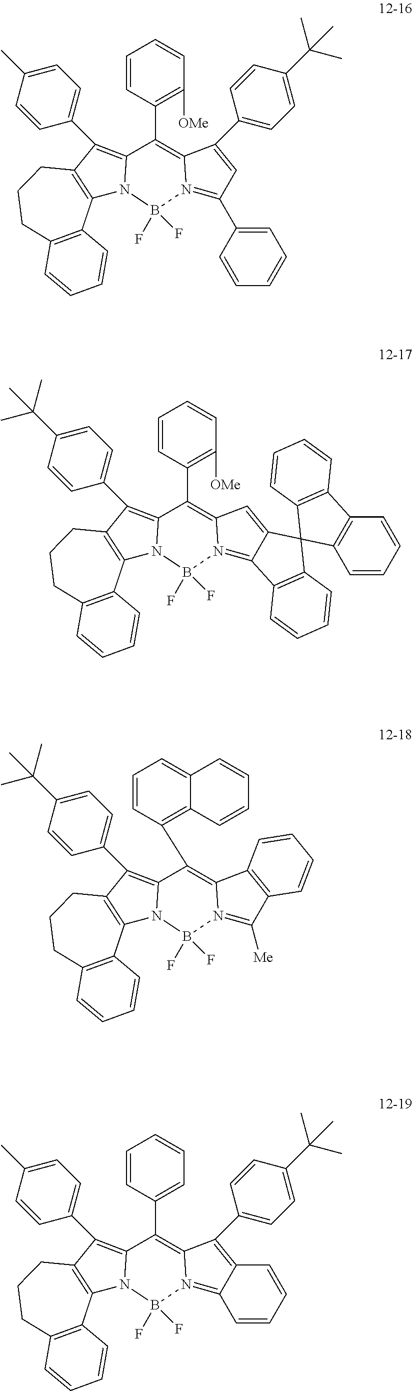

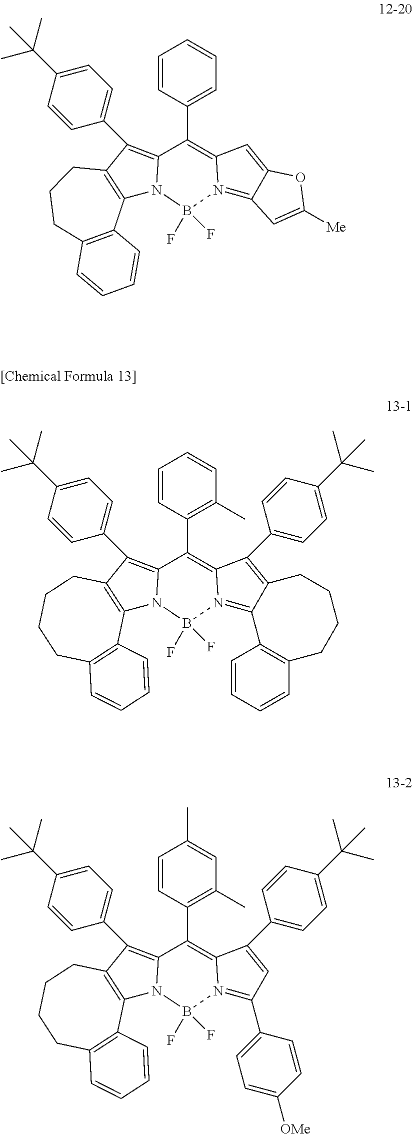

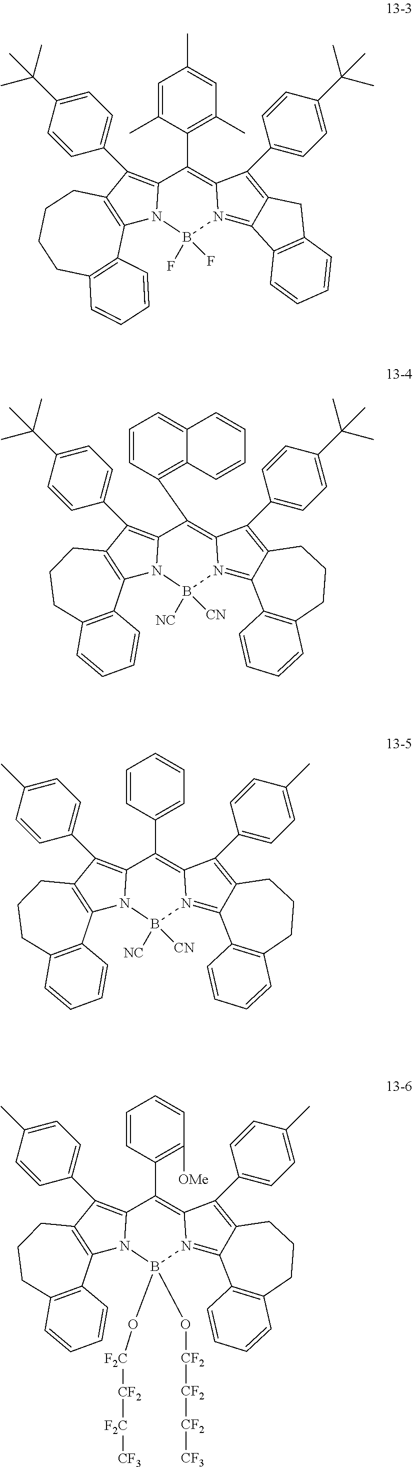

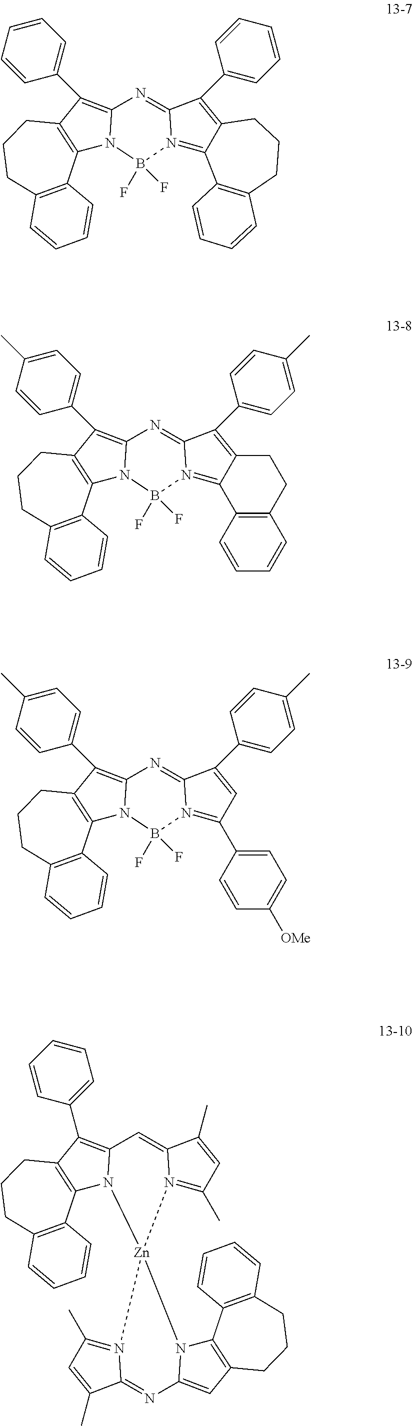

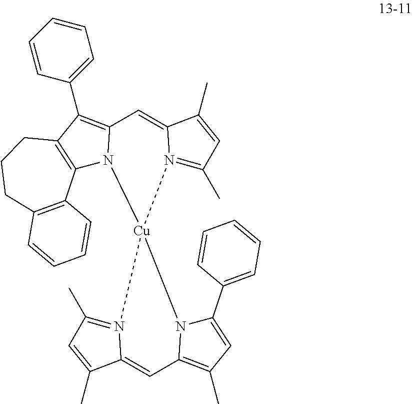

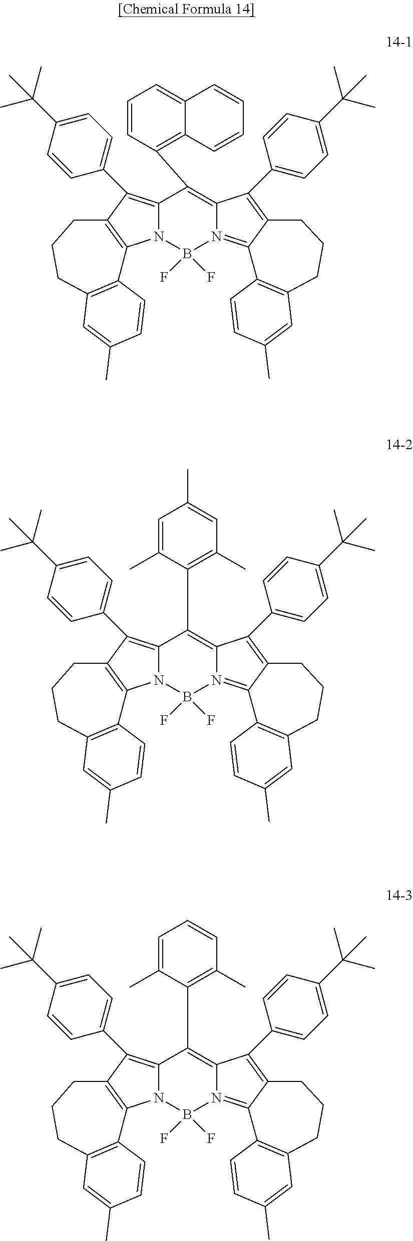

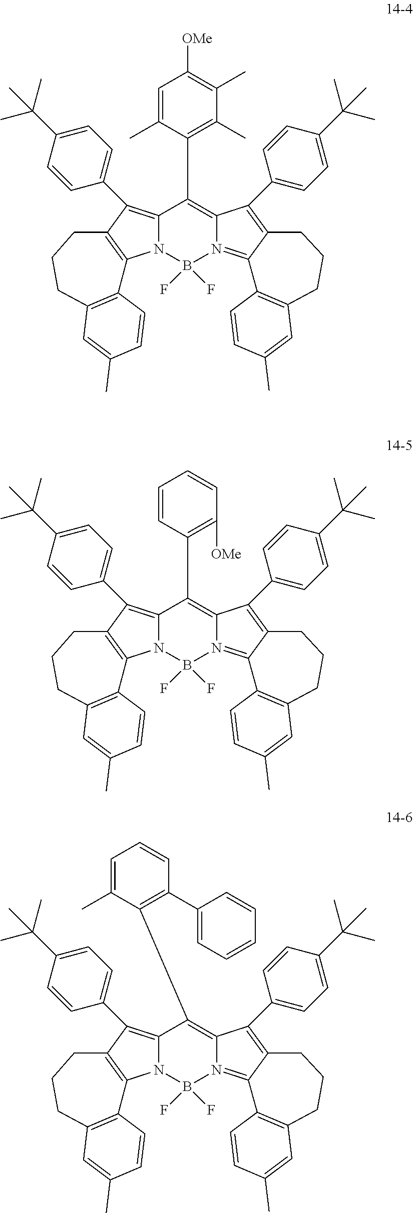

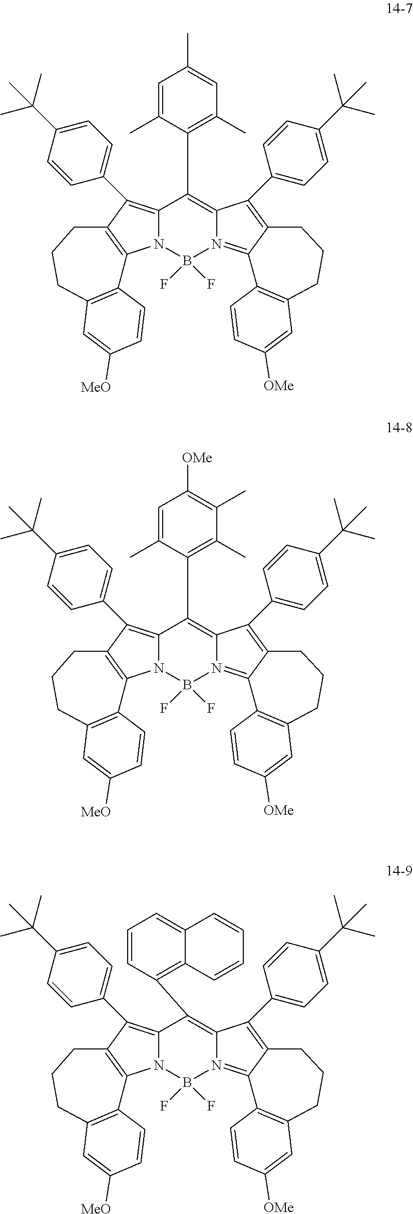

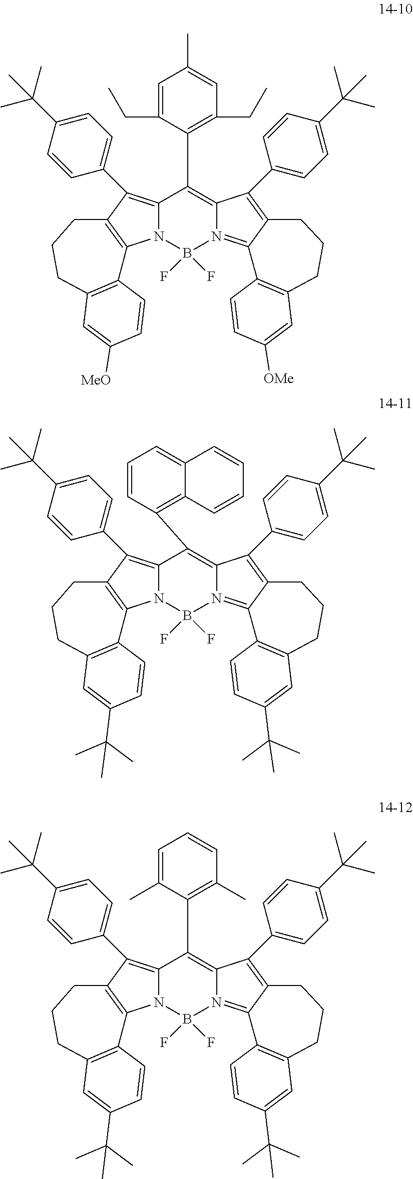

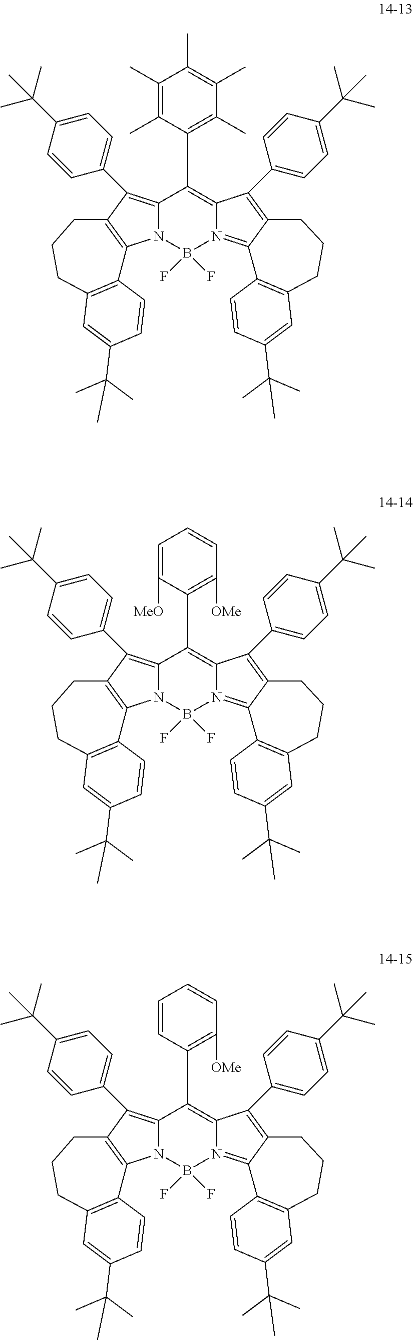

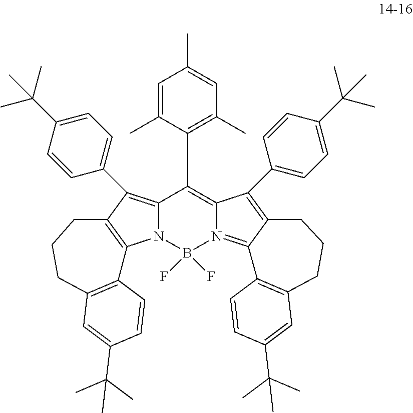

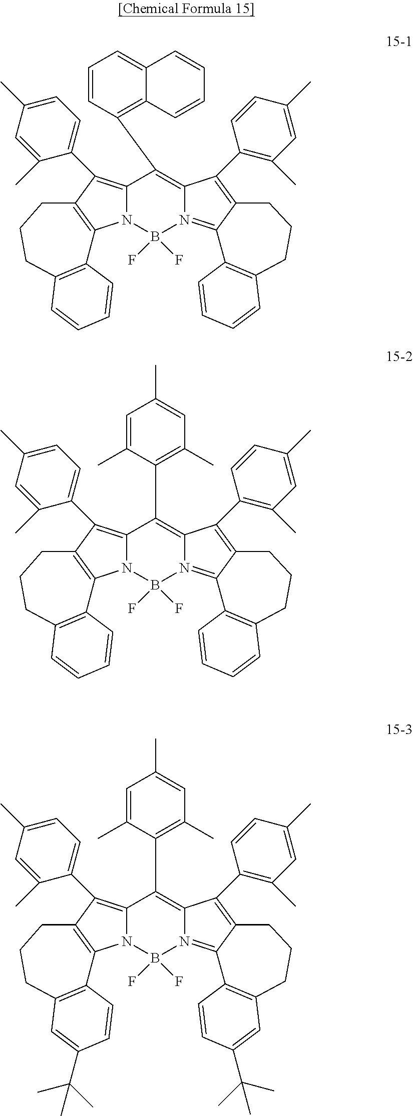

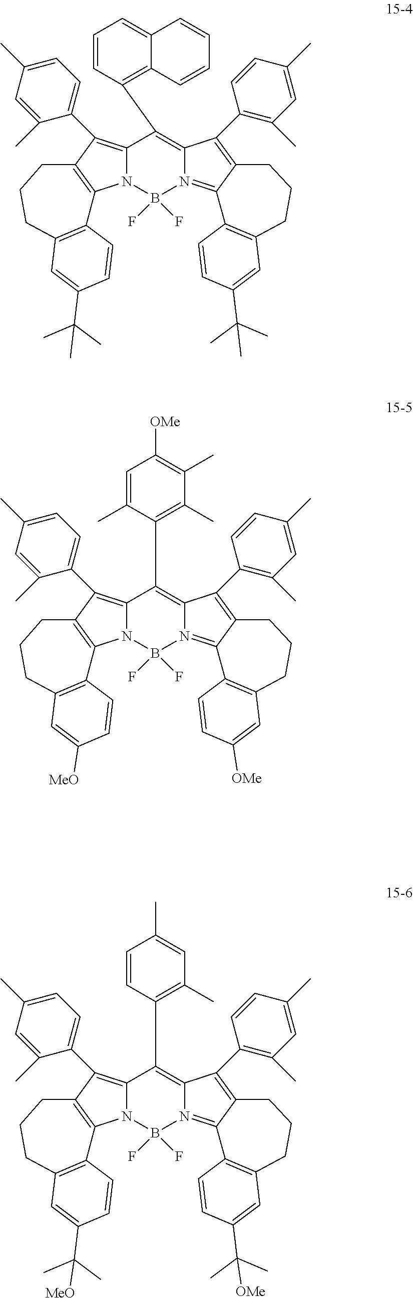

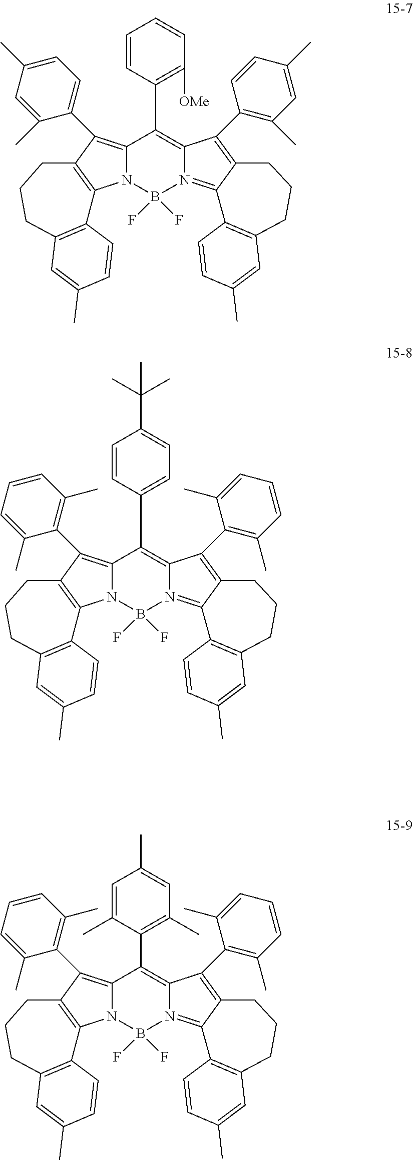

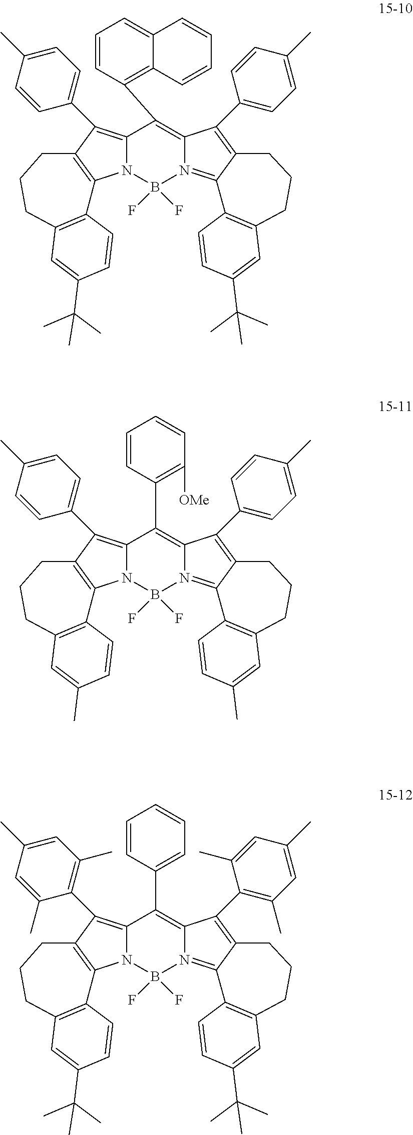

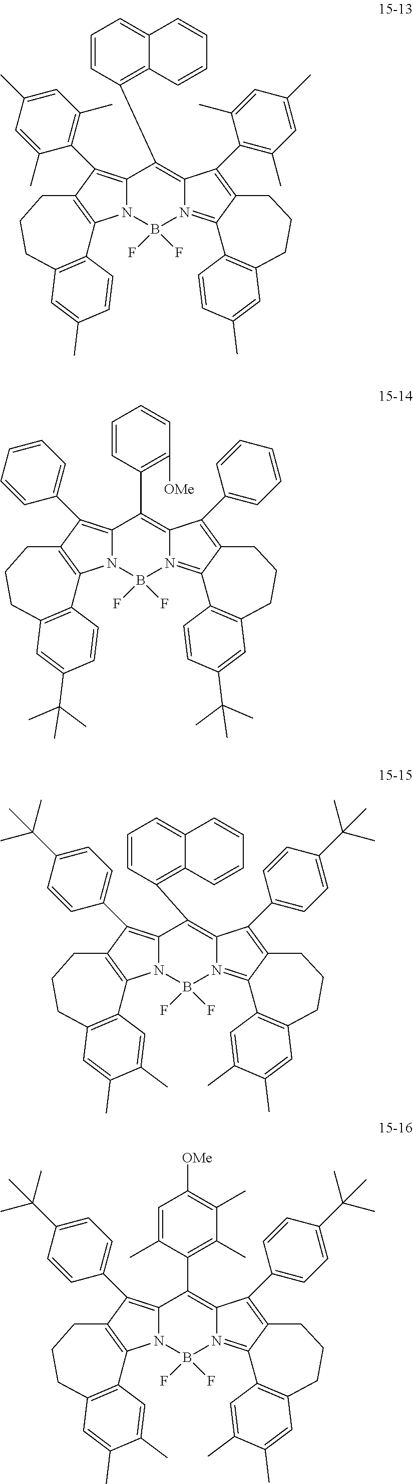

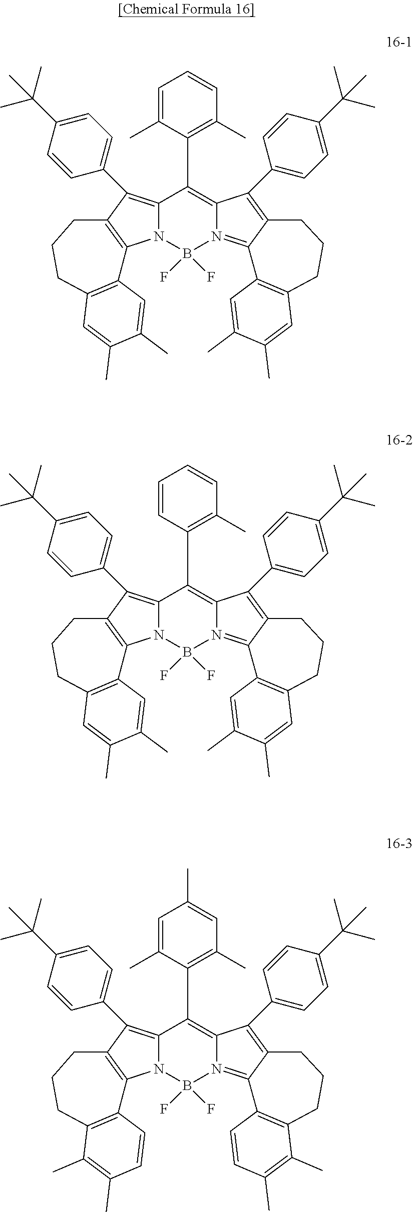

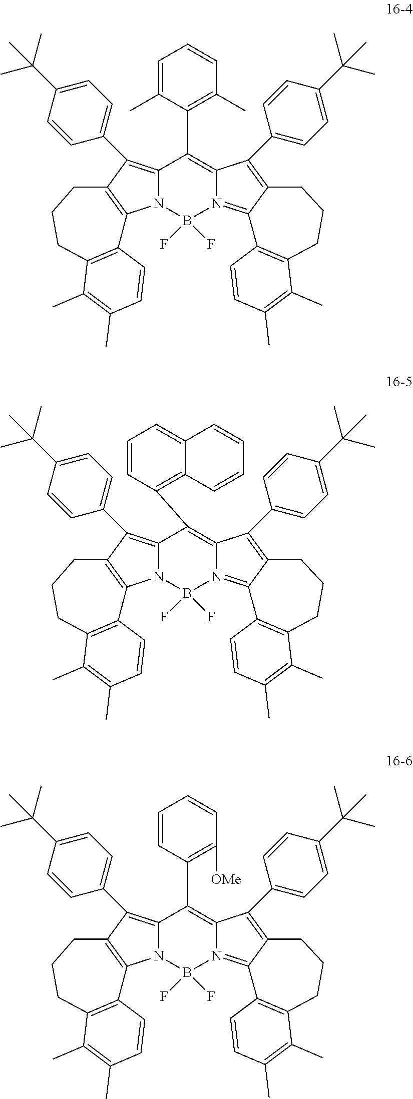

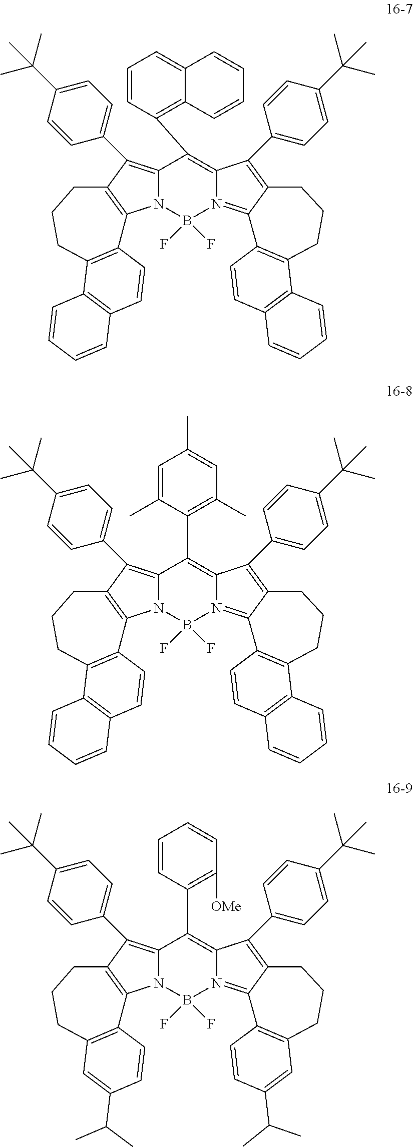

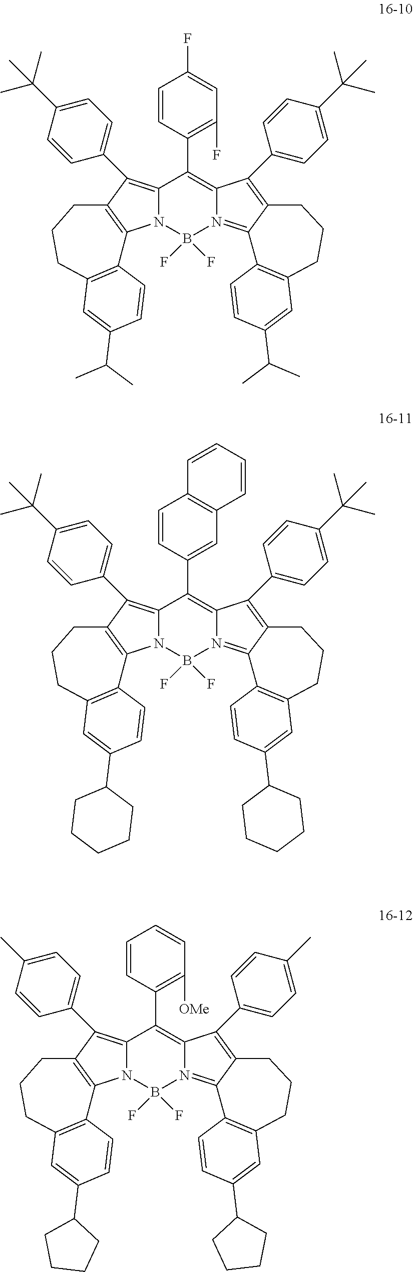

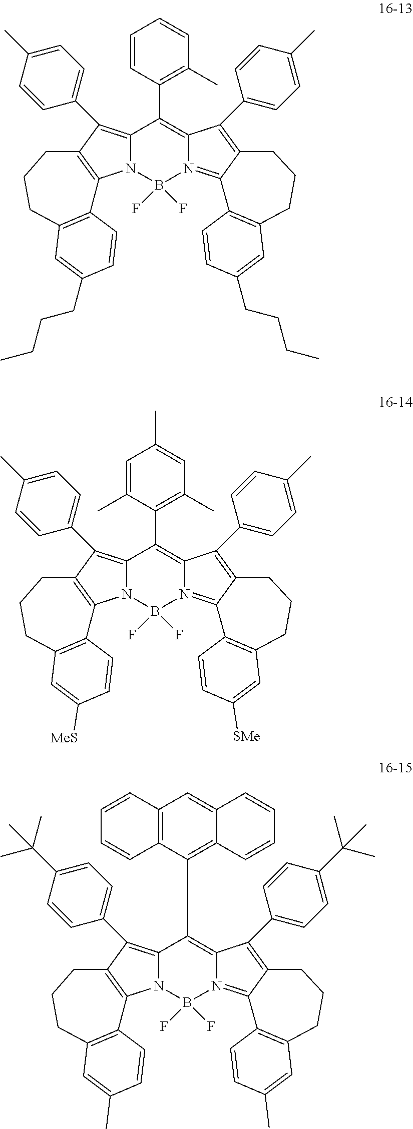

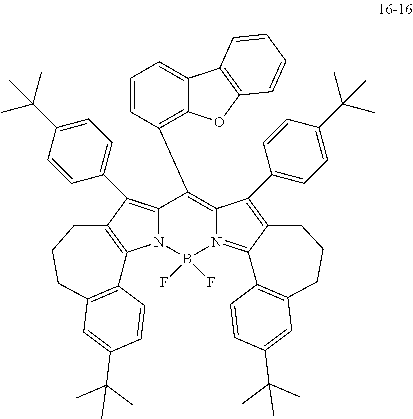

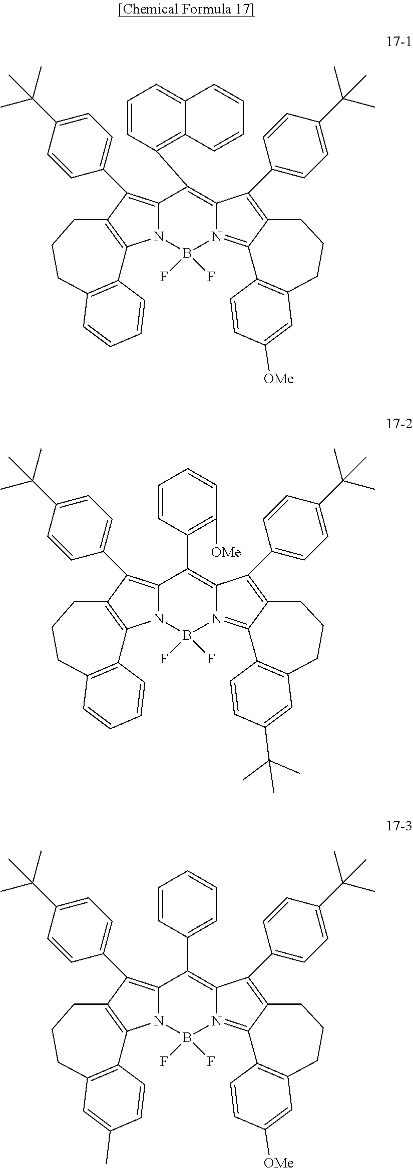

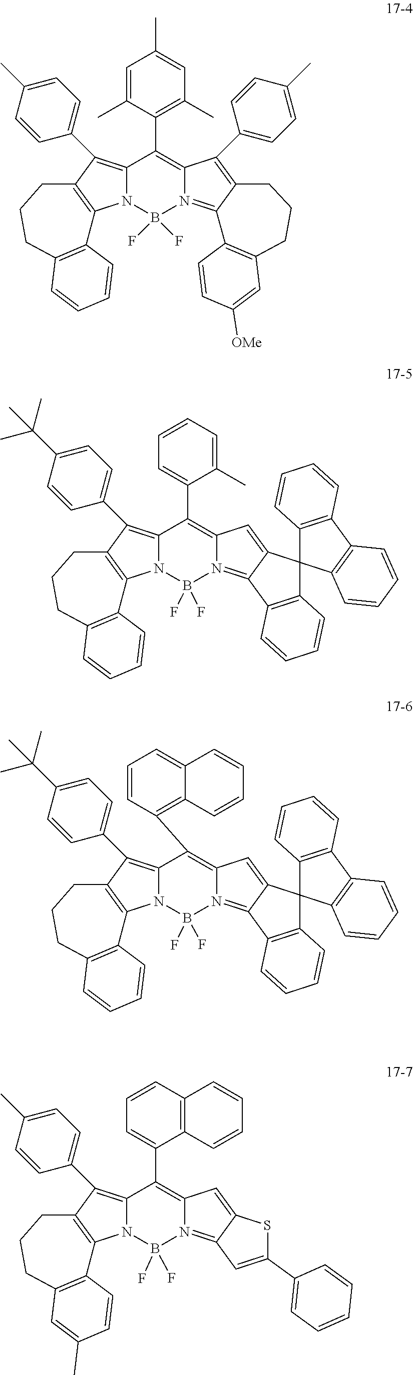

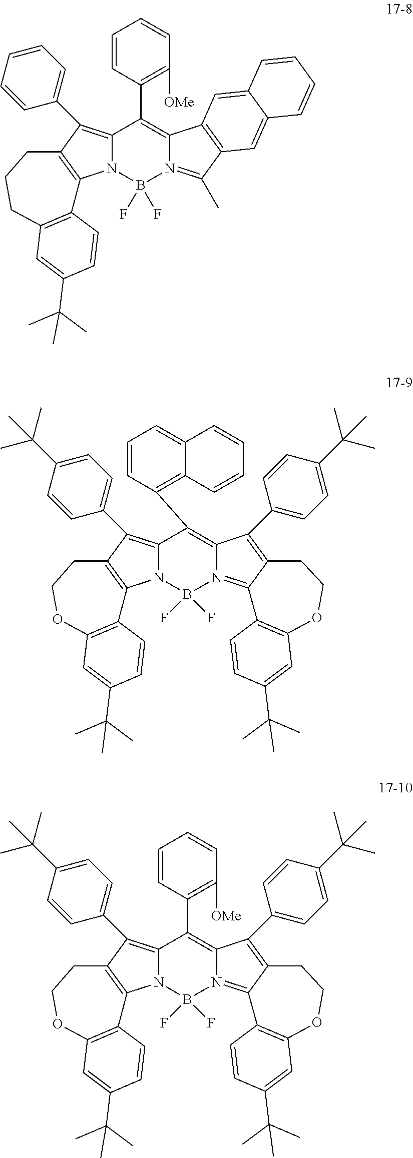

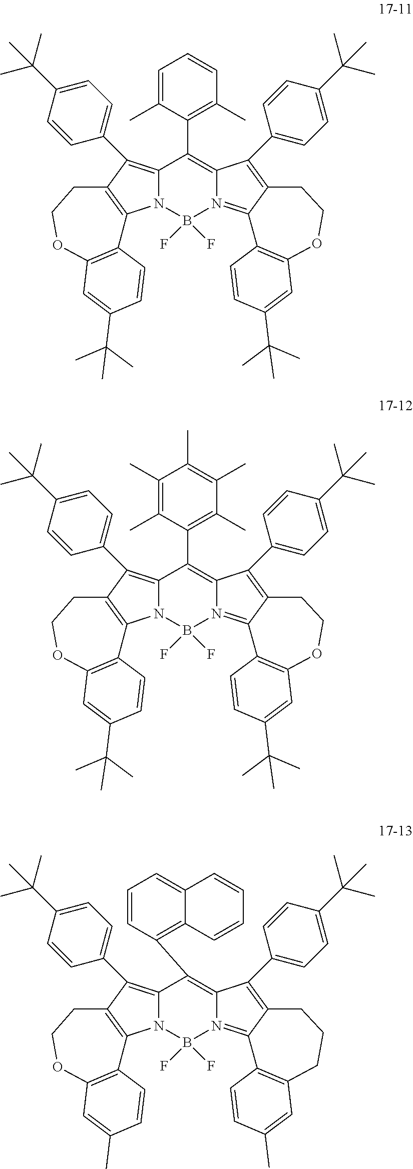

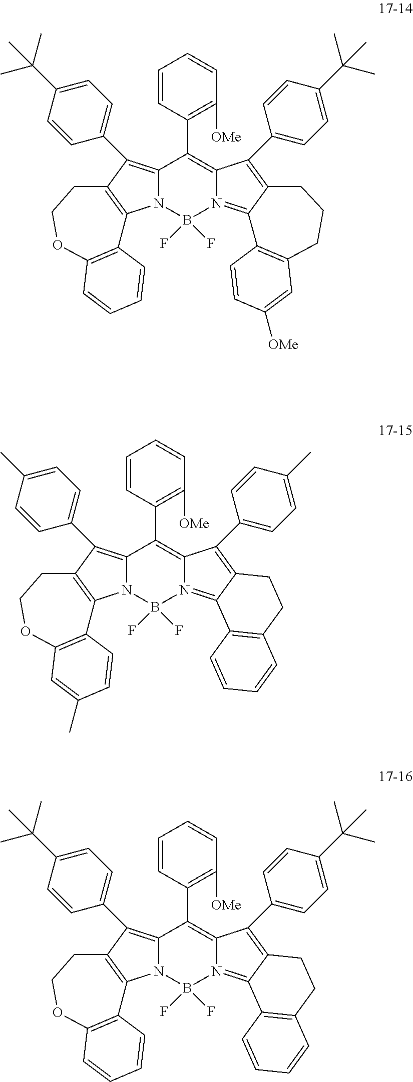

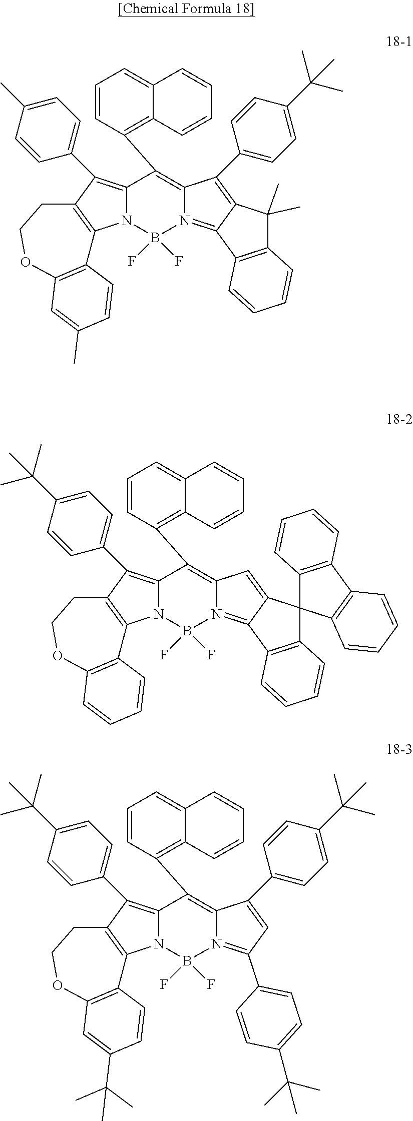

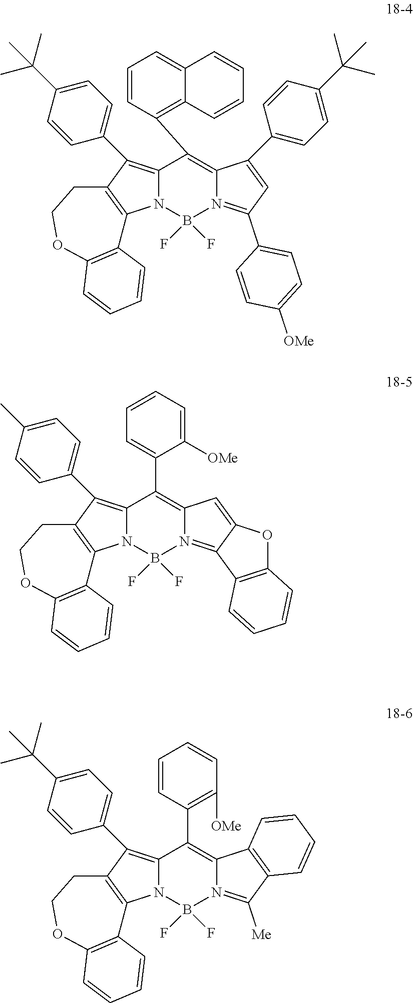

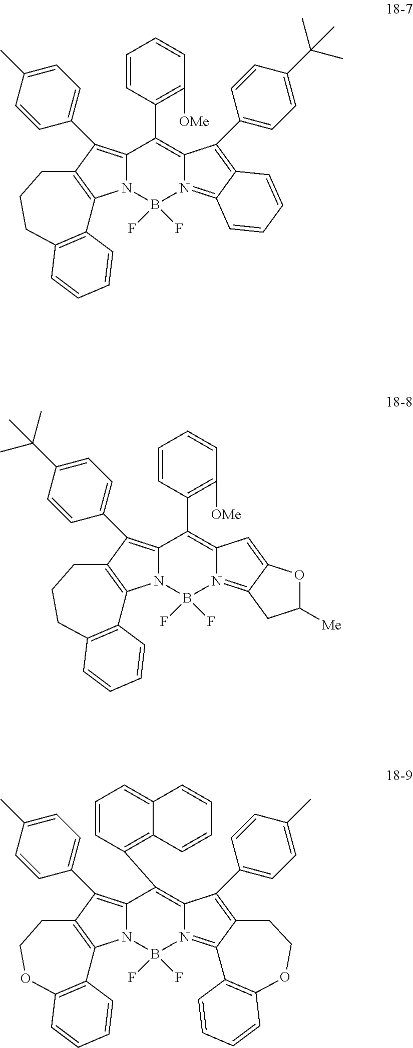

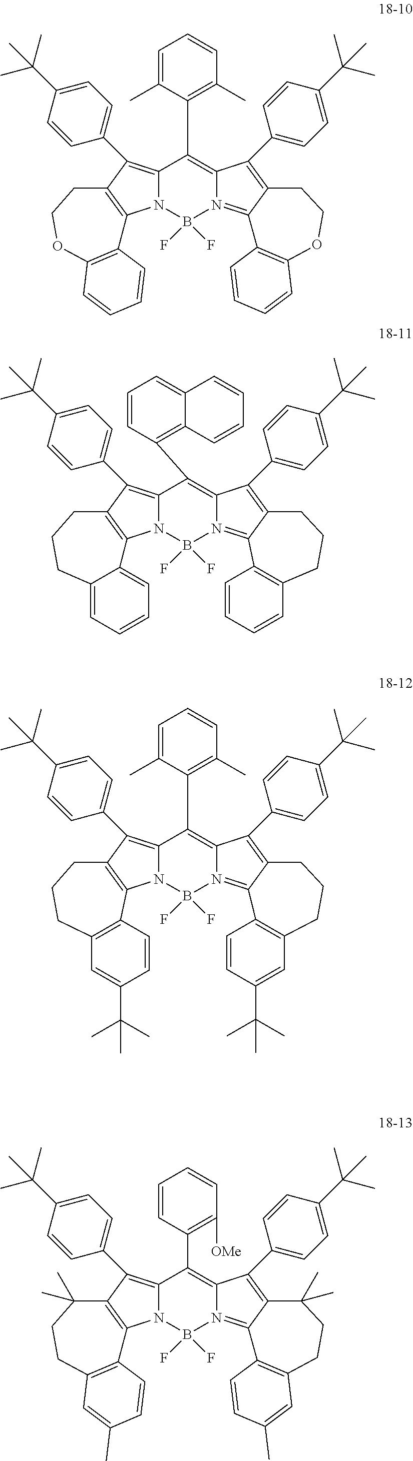

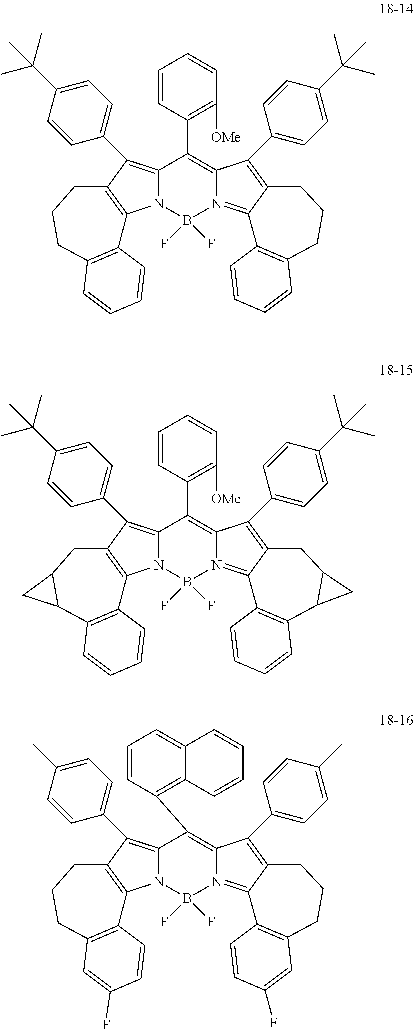

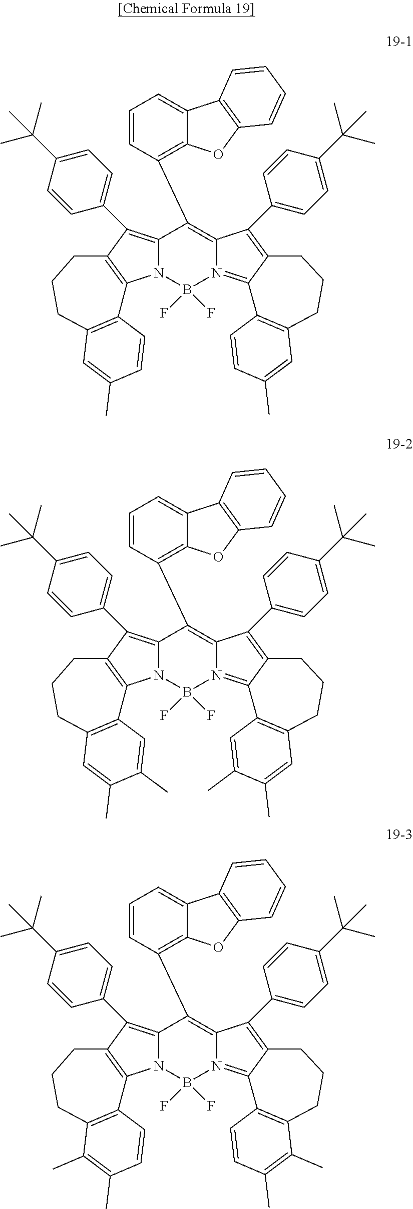

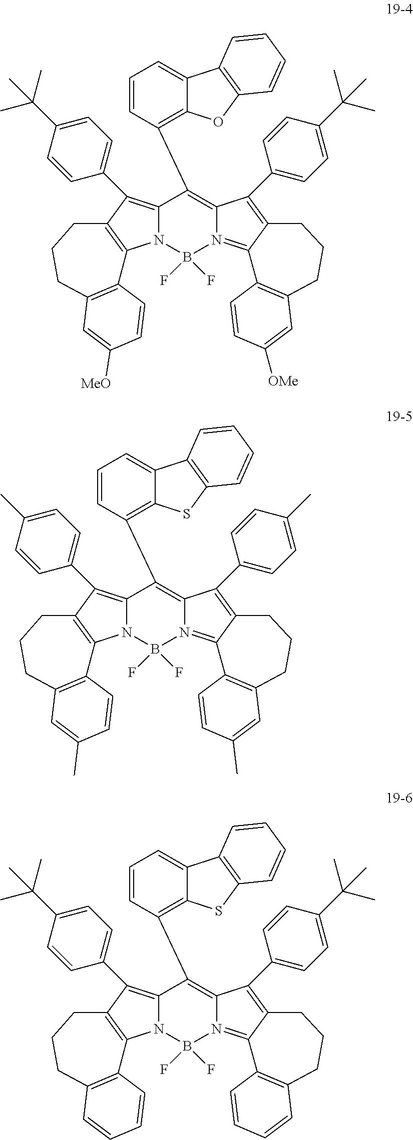

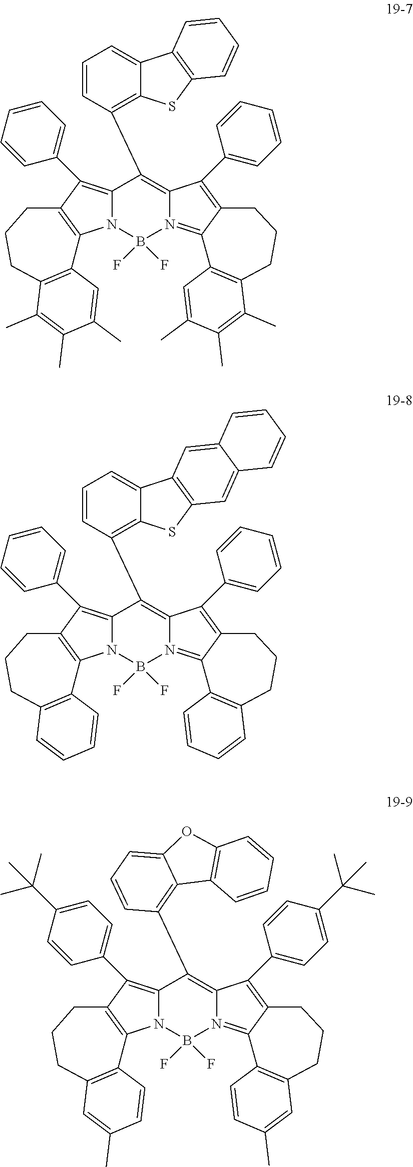

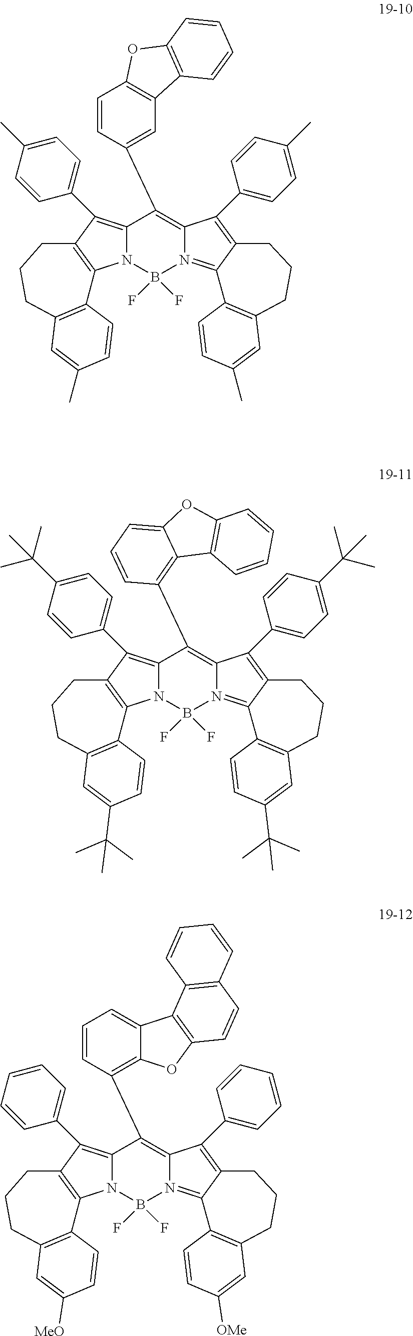

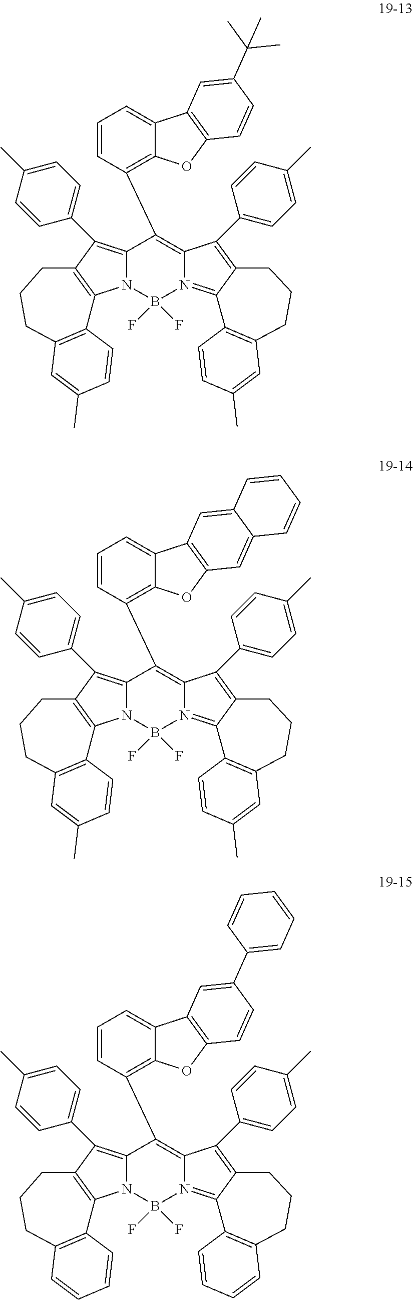

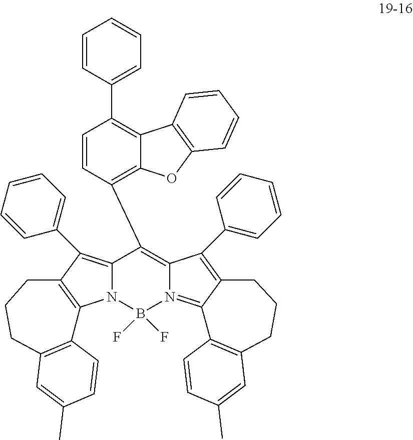

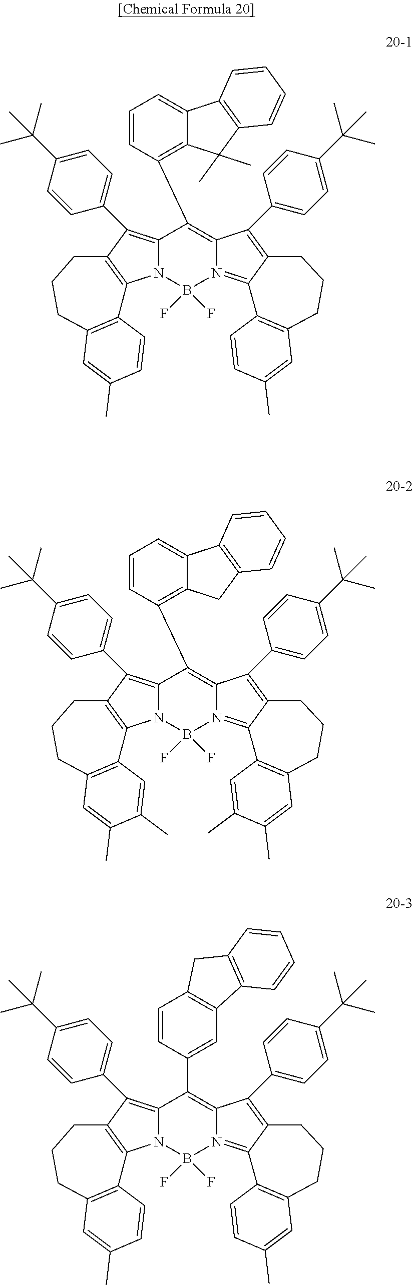

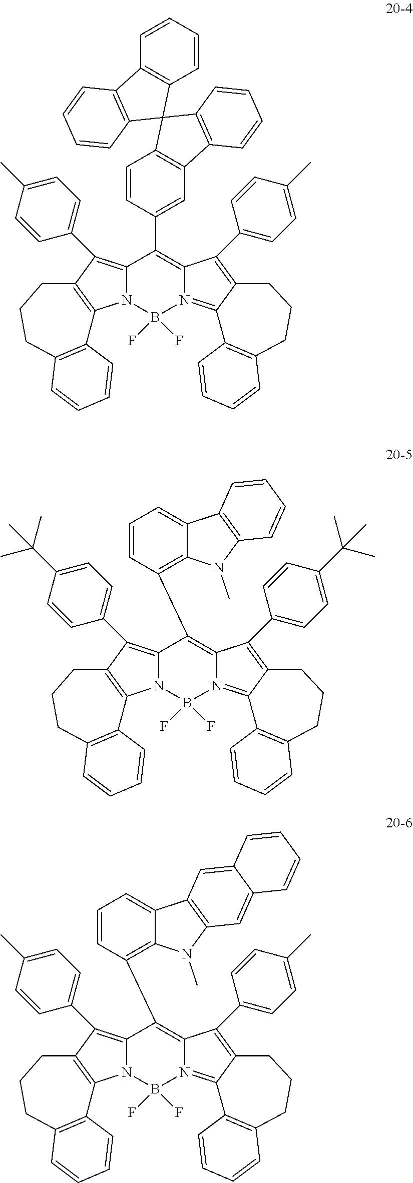

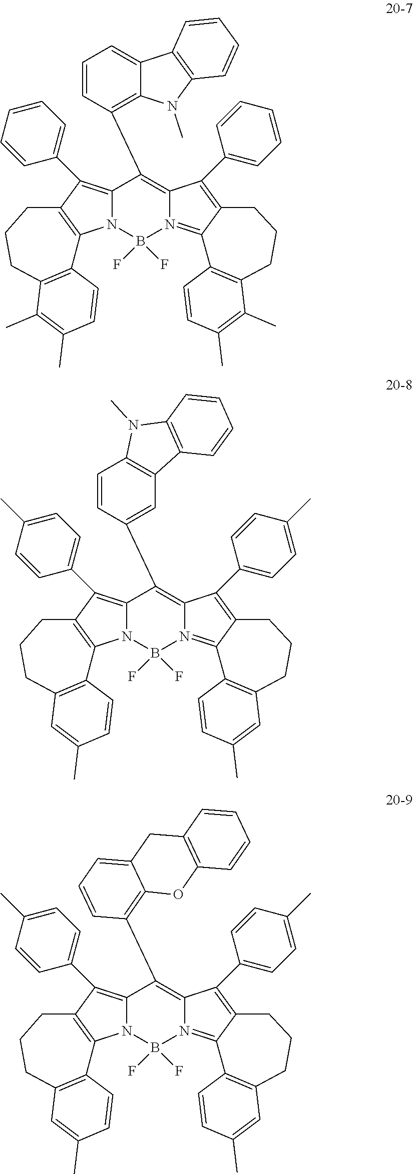

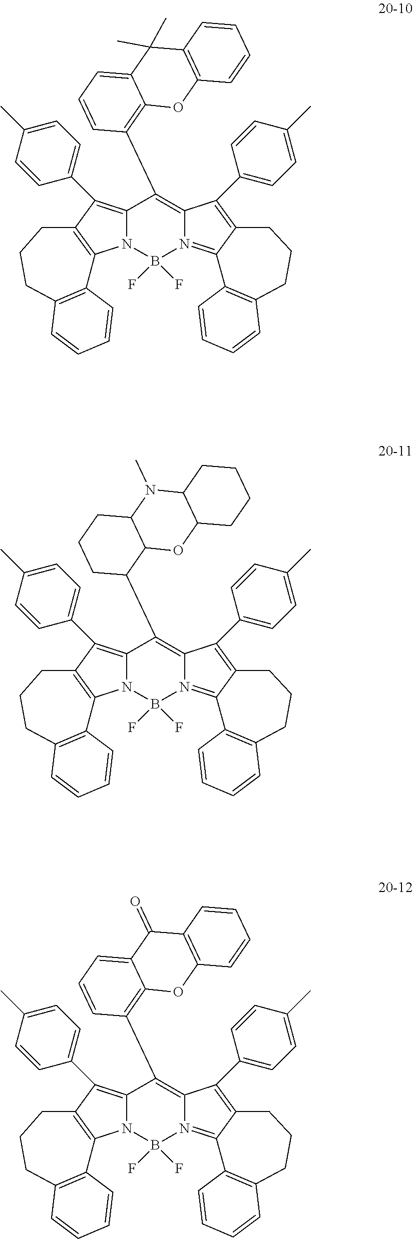

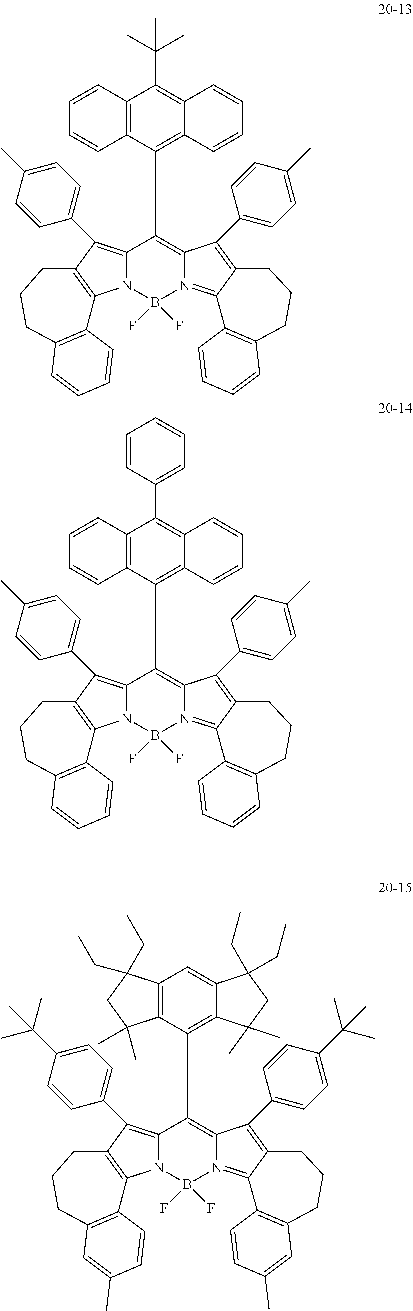

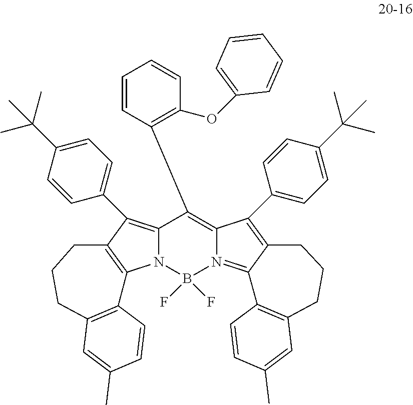

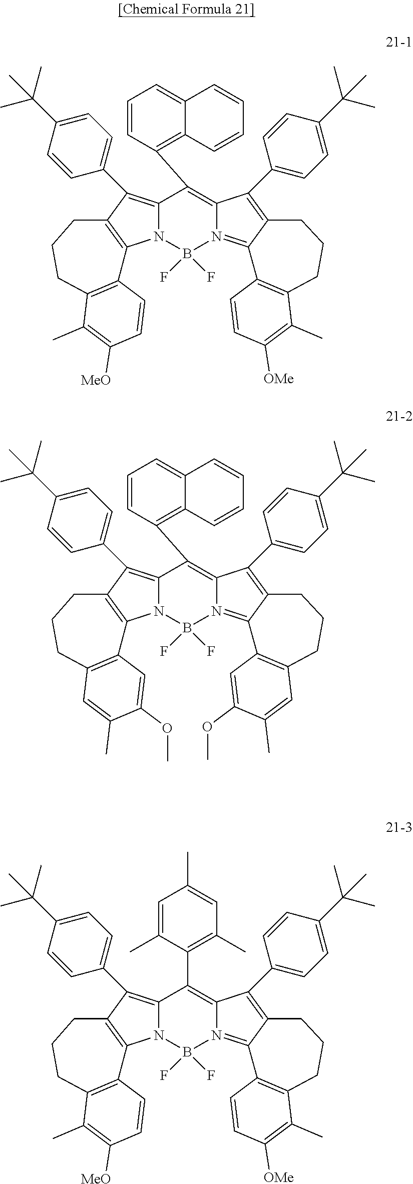

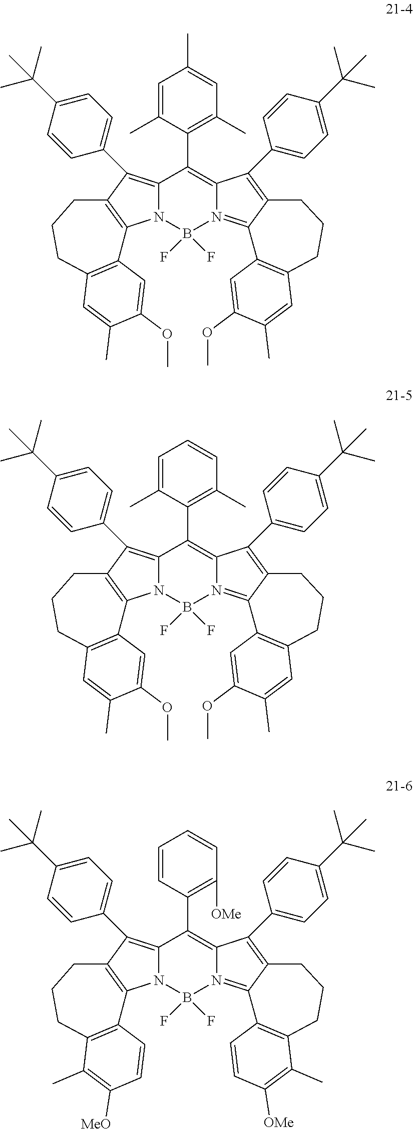

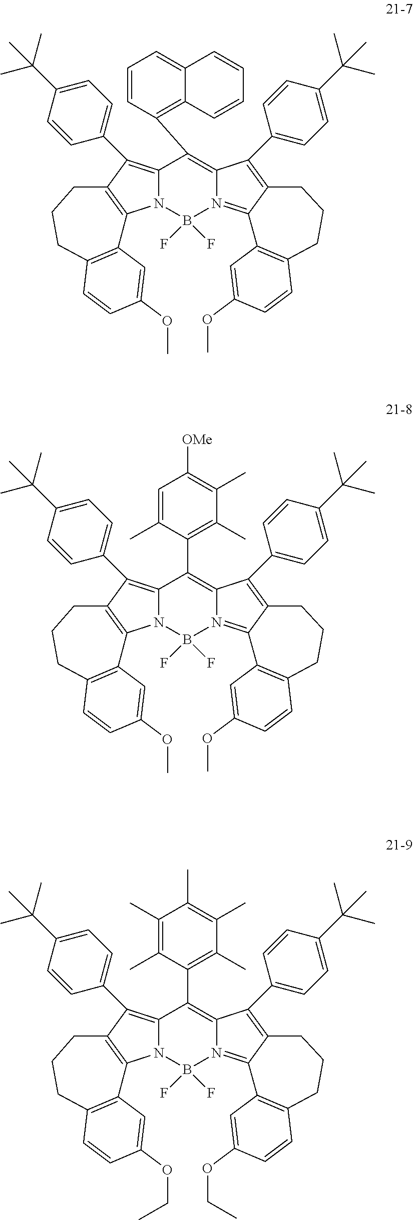

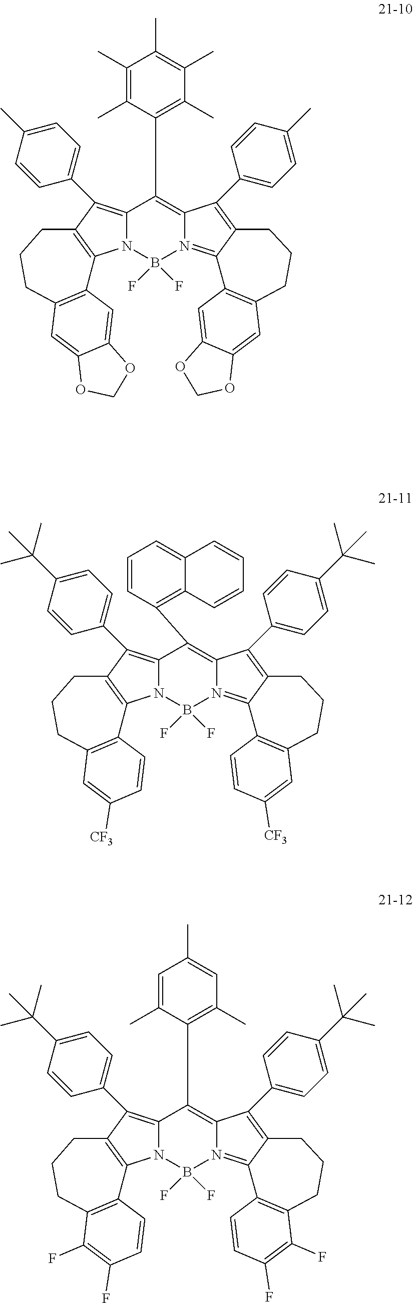

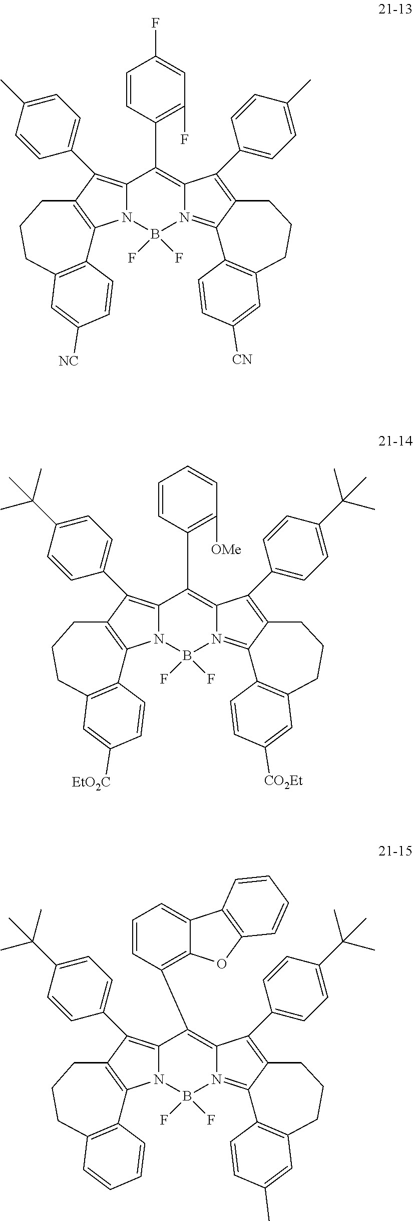

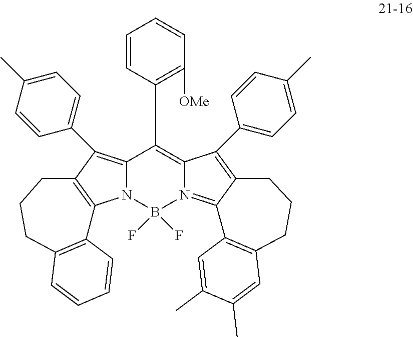

<Pyrromethene Compound>

The compounds before complex formation of the pyrromethene metal complex represented by the general formulae (1) and (2) are, for example, pyrromethene compounds represented by the general formulae (8) and (9), respectively.

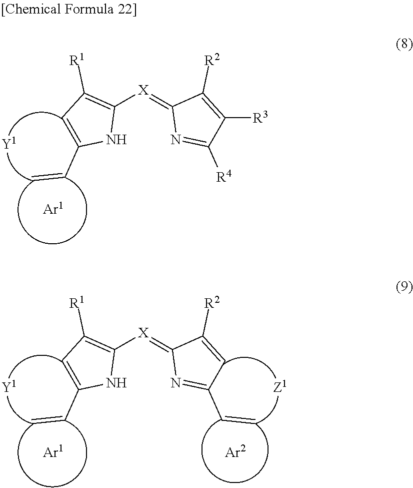

The general formulae (8) and (9) are the same as the general formulae (1) and (2), respectively, except that no complex is formed in the general formulae (8) and (9). The detailed description of X, R¹ to R⁵, Ar¹ to Ar², Y¹, and Z¹ is the same as that in the general formulae (1) and (2).

The pyrromethene metal complex represented by the general formula (1) or (2) can be produced with reference to methods described in J. Org. Chem., vol. 64, No. 21, pp. 7813-7819 (1999), Angew. Chem., Int. Ed. Engl., vol. 36, pp. 1333-1335 (1997), Org. Lett., vol. 12, pp. 296 (2010), and the like.

Specific examples of the method for producing a pyrromethene metal complex will be described below, but the method is not limited thereto.

A compound represented by the following general formula (10) and a compound represented by the general formula (11A) or (11B) are heated in 1,2-dichloroethane under the presence of phosphorus oxychloride to obtain a pyrromethene compound as a compound before complex formation. Next, the obtained pyrromethene compound is reacted with a metal compound represented by the following general formula (12) in 1,2-dichloroethane under the presence of triethylamine to obtain a target pyrromethene metal complex. Here, R¹ to R⁵, Ar¹, Ar², Y¹, Z¹, M, L, and m are the same as described above. J represents a halogen.

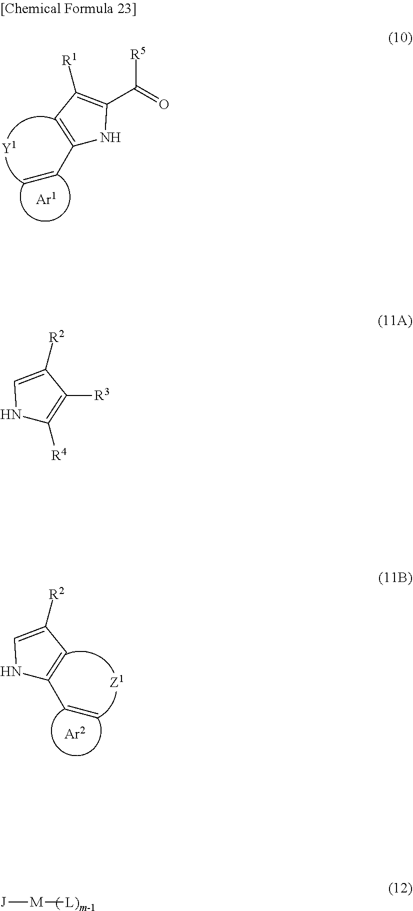

Examples of the method of introducing an aryl group or a heteroaryl group into a pyrromethene skeleton include, but are not limited to, a method in which a carbon-carbon bond is generated using a coupling reaction between a halogenated derivative of a pyrromethene compound and a boronic acid or a boronic acid ester derivative under the presence of a metal catalyst such as palladium. Similarly, examples of the method of introducing an amino group or a carbazolyl group into a pyrromethene skeleton include, but are not limited to, a method in which a carbon-nitrogen bond is generated using a coupling reaction between a halogenated derivative of a pyrromethene compound and an amine or a carbazole derivative under the presence of a metal catalyst such as palladium.

The pyrromethene metal complex represented by the general formula (1) or (2) is produced by, for example, reacting the pyrromethene compound with a metal halide. It is preferable to improve the purity of the obtained pyrromethene metal complex by organic synthetic purification such as recrystallization or column chromatography followed by purification by heating under reduce pressure, which is generally called sublimation purification, for removal of a low-boiling-point component. The heating temperature in the sublimation purification is not particularly limited, but is preferably 330° C. or less, and more preferably 300° C. or less from the viewpoint of preventing thermal decomposition of the pyrromethene metal complex. Furthermore, the heating temperature is preferably 230° C. or more, and more preferably 250° C. or more from the viewpoint of facilitating control of the deposition rate during deposition.

The purity of the pyrromethene metal complex produced in such a way is preferably 99% by weight or more from the viewpoint that the light-emitting element can exhibit a stable characteristic.

The optical characteristic of the pyrromethene metal complex represented by the general formula (1) or (2) can be determined by measuring the absorption spectrum and the emission spectrum of the diluted solution. The solvent is not particularly limited as long as it dissolves the pyrromethene metal complex and is transparent so that the absorption spectrum of the solvent does not overlap with the absorption spectrum of the pyrromethene metal complex, and specific examples of the solvent include toluene. The concentration of the solution is not particularly limited as long as the solution has sufficient absorbance and does not cause concentration quenching, and is preferably in the range of 1×10⁻⁴ mol/L to 1×10⁻⁷ mol/L, and more preferably in the range of 1×10⁻⁵ mol/L to 1×10⁻⁶ mol/L. The absorption spectrum can be measured with a general ultraviolet-visible spectrophotometer. The emission spectrum can be measured with a general fluorescence spectrophotometer. The fluorescence quantum yield is preferably measured using an absolute quantum yield measurement device in which an integrating sphere is used.

The pyrromethene metal complex represented by the general formula (1) or (2) preferably emits light by using excitation light so that the peak wavelength is observed in the region of 580 nm or more and 750 nm or less. Hereinafter, light emission in which the peak wavelength is observed in the region of 580 nm or more and 750 nm or less is referred to as “emission of red light”.

In the case that the pyrromethene metal complex of the present invention is used in a display device or an illumination device, the peak wavelength is preferably in the region of 600 nm or more and 640 nm or less, and more preferably in the region of 600 nm or more and 630 nm or less from the viewpoint of expanding the color gamut to improve the color reproducibility.

Meanwhile, in the case that the pyrromethene metal complex of the present invention is used as a fluorescent probe for bioimaging, the peak wavelength of the emission spectrum is preferably 650 to 750 nm, and more preferably 700 to 750 nm from the viewpoints of small absorption and high transmittance in the living body.

The pyrromethene metal complex represented by the general formula (1) or (2) preferably emits red light by using excitation light having a wavelength in the range of 430 nm or more and 600 nm or less. When the pyrromethene metal complex represented by the general formula (1) or (2) is used as a dopant material of a light-emitting element, the pyrromethene metal complex emits red light by absorbing light emitted from the host material. A general host material emits light in the wavelength range of 430 nm or more and 580 nm or less, so that achievement of emission of red light with the excitation light contributes to improvement in the efficiency of the light-emitting element.

In the case that the pyrromethene metal complex represented by the general formula (1) or (2) is used in a display device or an illumination device, light emitted through irradiation with excitation light preferably has a sharp emission spectrum for achievement of high color purity. From this viewpoint, the half-value width of the emission spectrum is preferably 40 nm or less.

Meanwhile, in the case that the pyrromethene metal complex according to embodiments of the present invention is used as a fluorescent probe for bioimaging, a plurality of fluorescent probes can be simultaneously evaluated because a fluorescent probe species having an emission spectrum having a narrow half-value width is easy to separate. From this viewpoint, the half-value width of the emission spectrum is preferably 40 nm or less as described above.

The luminous efficiency of the light-emitting element depends on the fluorescence quantum yield of the emissive material itself. Therefore, the fluorescence quantum yield is desired to be as close as possible to 100%. From the above viewpoint, the fluorescence quantum yield of the pyrromethene metal complex of the present invention is preferably 90% or more, and more preferably 95% or more. However, the fluorescence quantum yield shown here is determined through measuring a diluted solution using toluene as a solvent with an absolute quantum yield measurement device.

The pyrromethene metal complex represented by the general formula (1) or (2) is assumed to be used in a thin film form in a light-emitting element, particularly as a dopant. From the above, it is preferable to evaluate the optical characteristic of the thin film doped with the pyrromethene metal complex represented by the general formula (1) or (2) (hereinafter, referred to as a doped thin film).

The doped thin film is formed through co-depositing a matrix material and a pyrromethene metal complex represented by the general formula (1) or (2) on a transparent substrate having no absorption in a visible region. Here, as the matrix material, a wide band gap material having no absorption of excitation light is used, and specific examples include mCBP. The doping concentration with the pyrromethene metal complex represented by the general formula (1) or (2) is preferably equivalent to the doping concentration in the light-emitting element, and is preferably selected from the range of 0.1 to 20% by weight. The thickness of the doped thin film is not particularly limited as long as the doped thin film sufficiently absorbs excitation light and is easy to produce, and the thickness is preferably in the range of 100 to 1,000 nm. After the doped thin film is formed, it may be sealed with a transparent sealing resin.

The light emission wavelength from the doped thin film generally tends to be equivalent to or longer than that in the solution state. Therefore, the light emission peak wavelength of the doped thin film including the pyrromethene metal complex represented by the general formula (1) or (2) is preferably in the region of 580 nm or more and 750 nm or less, more preferably in the region of 600 nm or more and 650 nm or less, and still more preferably in the region of 600 nm or more and 640 nm or less.

The half-value width of the emission spectrum of the doped thin film generally tends to be equivalent to or larger than that in the solution state. Therefore, the half-value width of the emission spectrum of the doped thin film including the pyrromethene metal complex represented by the general formula (1) or (2) is preferably 50 nm or less, more preferably 45 nm or less, and still more preferably 40 nm or less.

The fluorescence quantum yield of the doped thin film can be measured using an absolute quantum yield measurement device. However, the fluorescence quantum yield varies under the influence of the formation state of the doped thin film, the combination with the matrix material, the excitation light wavelength, and the like, and therefore the absolute values of the fluorescence quantum yields are difficult to compare. Therefore, the fluorescence quantum yield of the doped thin film of each material is preferably measured under a certain condition and relatively compared for evaluation. In the doped thin film, a negative correlation is observed in which the fluorescence quantum yield decreases due to concentration quenching as the doping concentration increases. However, a high negative correlation is disadvantageous in production of a light-emitting element because the allowable range of the doping concentration is small when the negative correlation is high. Therefore, a material is preferable that has a low negative correlation between the fluorescence quantum yield and the doping concentration.

Here, in the doped thin film including the pyrromethene metal complex represented by the general formula (1) or (2) in which R⁵ is represented by the general formula (6), the steric hindrance of the substituent at the bridge-head position suppresses rotation and vibration of the molecule to reduce the heat deactivation, and as a result, a high fluorescence quantum yield can be obtained. Furthermore, concentration quenching rarely occurs because flocculation of the molecules is suppressed due to the influence of the steric hindrance of the substituent at the bridge-head position and because even if self-absorption of the emitted light occurs, non-radiative deactivation is small due to the high fluorescence quantum yield of the pyrromethene boron complex itself, and as a result, the negative correlation between the fluorescence quantum yield and the doping concentration can be low.

In addition, the molecular orientation can be measured through examining the angle dependence of the emission spectrum of the doped thin film. The emission from the dopant molecule itself has angle dependence, so that in the doped thin film, the radiation intensity of light to a certain angle is higher in the case that the dopant molecules are present in a state of being aligned in a certain direction, that is, the dopant molecules are oriented than in the case that the dopant molecules are present in random directions. With regard to a light-emitting element having such a doped thin film, if the angle at which the radiation intensity increases is aligned with the direction in which light is taken out, it is possible to increase the amount of light taken out to the outside to improve the luminous efficiency of the element. In particular in a top emission element in which the resonance effect is utilized, the direction in which light is taken out is limited, and therefore the molecular orientation of the doped thin film is preferably enhanced from the viewpoint of improving the luminous efficiency. The pyrromethene metal complex represented by the general formula (1) or (2) in which R⁵ is represented by the general formula (6) has a rigid structure in which the steric hindrance of the substituent at the bridge-head position suppresses rotation and vibration of each molecular, and as a result, such pyrromethene metal complexes are aligned more easily than molecules having a flexible structure, and the molecular orientation of the doped thin film can be enhanced.

<Light-Emitting Element Material>

The pyrromethene metal complex represented by the general formula (1) or (2) can achieve both high luminous efficiency and high color purity, and therefore is preferably used as an electronic device material in an electronic device, and particularly preferably used as a light-emitting element material in a light-emitting element. Here, the term “light-emitting element material” in the present invention refers to a material used in any layer of the light-emitting element, and as described below, refers to a material used in a hole injection layer, a hole transporting layer, an emissive layer and/or an electron transporting layer, and also refers to a material used in a protective film (cap layer) of an electrode.

The pyrromethene metal complex represented by the general formula (1) or (2) has high light emission performance, and therefore is preferably a material used in an emissive layer. The pyrromethene metal complex represented by the general formula (1) or (2) exhibits strong emission particularly in a red region, and therefore is suitably used as a red-light-emitting material.

Furthermore, a white-light-emitting element can be obtained by stacking an emissive layer including the pyrromethene metal complex represented by the general formula (1) or (2), an emissive layer including a blue-light-emitting material, and an emissive layer including a green-light-emitting material.

The light-emitting element material of the present invention may include the pyrromethene metal complex represented by the general formula (1) or (2) alone, or may include a mixture containing the pyrromethene metal complex and a plurality of other compounds. From the viewpoint of stable production of the light-emitting element, the light-emitting element material preferably includes the pyrromethene metal complex represented by the general formula (1) or (2) alone. Here, the phrase “the pyrromethene metal complex represented by the general formula (1) or (2) alone” means that the compound is included at a content of 99% by weight or more.

<Light-Emitting Element>

Next, embodiments of the light-emitting element of the present invention will be described. The light-emitting element according to embodiments of the present invention includes an anode, a cathode, and an organic layer between the anode and the cathode, the organic layer includes at least an emissive layer, and the emissive layer emits light by electrical energy. The light-emitting element according to embodiments of the present invention includes the pyrromethene metal complex represented by the general formula (1) or (2) in the emissive layer.

The light-emitting element of the present invention may be a bottom emission type or top emission type light-emitting element.

Examples of the layer configuration between the anode and the cathode in such a light-emitting element include, other than the configuration of only an emissive layer, laminated configurations such as 1) an emissive layer/an electron transporting layer, 2) a hole transporting layer/an emissive layer, 3) a hole transporting layer/an emissive layer/an electron transporting layer, 4) a hole injection layer/a hole transporting layer/an emissive layer/an electron transporting layer, 5) a hole transporting layer/an emissive layer/an electron transporting layer/an electron injection layer, 6) a hole injection layer/a hole transporting layer/an emissive layer/an electron transporting layer/an electron injection layer, 7) a hole injection layer/a hole transporting layer/an emissive layer/a hole inhibition layer/an electron transporting layer/an electron injection layer, 8) a hole injection layer/a hole transporting layer/an electron inhibition layer/an emissive layer/a hole inhibition layer/an electron transporting layer/an electron injection layer.

The light-emitting element may be a tandem type light-emitting element in which a plurality of the above-described laminated configurations are stacked with an intermediate layer interposed therebetween. That is, at least two emissive layers are preferably provided between the anode and the cathode, and at least one charge generation layer is preferably interposed between the at least two emissive layers. Here, in the case that at least two emissive layers are provided, at least one of the emissive layers includes the pyrromethene metal complex represented by the general formula (1) or (2). That is, in the case that a plurality of emissive layers are provided, all of the plurality of emissive layers or only a part of the plurality of emissive layers may include the pyrromethene metal complex represented by the general formula (1) or (2). The tandem type element has characteristics of high efficiency and long life because the plurality of emissive layers in the tandem type element enables to achieve high luminance with a low current. Furthermore, a light-emitting element including emissive layers of three colors of red (R), green (G), and blue (B) is a white light element that is highly efficient and mainly used in fields of television and illumination. The method in which such a white light element is used also has an advantage that the process can be simpler than that in a method of painting RGB colors. Examples of the intermediate layer generally include an intermediate electrode, an intermediate electroconductive layer, a charge generation layer, an electron draw-out layer, a connection layer, and an intermediate insulating layer, and a known material configuration can be used. Preferred specific examples of the tandem type light-emitting element include a light-emitting element having a laminated configuration including a charge generation layer as an intermediate layer between an anode and a cathode, such as 9) a hole transporting layer/an emissive layer/an electron transporting layer/a charge generation layer/a hole transporting layer/an emissive layer/an electron transporting layer, or 10) a hole injection layer/a hole transporting layer/an emissive layer/an electron transporting layer/an electron injection layer/a charge generation layer/a hole injection layer/a hole transporting layer/an emissive layer/an electron transporting layer/an electron injection layer. As the specific material included in the intermediate layer, a pyridine derivative and a phenanthroline derivative are preferably used.

Each of the above-described layers may be a single layer or a layer including a plurality of layers, and may be doped. Furthermore, examples of the configuration include an element configuration including an anode, one or more organic layers including an emissive layer, a cathode, and a layer in which a capping material is used for improving luminous efficiency due to an optical interference effect.

The pyrromethene metal complex represented by the general formula (1) or (2) may be used in any layer in the above-described element configuration, and is preferably used in the emissive layer because the pyrromethene metal complex has a high fluorescence quantum yield and thin film stability.

The light-emitting element of the present invention is preferably a top emission type organic electroluminescent element. In the case of a top emission type organic electroluminescent element, for example, there is a method in which the anode has a laminated structure in which a reflective electrode layer and a transparent electrode layer are stacked, and the thickness of the transparent electrode layer on the reflective electrode layer is varied. In the method, the anode is appropriately laminated with an organic layer, then, for example, a translucent silver thin film is used in the cathode as a translucent electrode, and thus, a microcavity structure can be introduced into the organic electroluminescent element. As described above, if the microcavity structure is introduced into the organic electroluminescent element, the spectrum of the light emitted from the organic layer through the cathode is sharper than that in a case where the organic electroluminescent element does not have the microcavity structure, and the emission intensity to the front is greatly increased. In such a top emission type element, the luminous efficiency can be further enhanced with an emissive material having a sharp emission spectrum due to the microcavity effect, and therefore, the emissive material according to an embodiment of the present invention is particularly effective. When used in a display, the emissive material can contribute to improvement in the color gamut and improvement in the luminance.

Specific examples of the configuration of the light-emitting element will be described below, but the configuration of the present invention is not limited thereto.

(Substrate)

The light-emitting element is preferably formed on a substrate in order to maintain the mechanical strength of the light-emitting element. As the substrate, a glass substrate such as a soda glass substrate or an alkali-free glass substrate is suitably used. The glass substrate is to have a thickness of 0.5 mm or more as a sufficient thickness to maintain the mechanical strength. The material of the glass is preferably alkali-free glass so that the amount of an ion eluted from the glass is small. Barrier coated soda lime glass coated with SiO₂ or the like is commercially available, and can also be used. The substrate is not necessarily to be glass as long as the first electrode formed on the substrate functions stably, and may be, for example, a plastic substrate. Examples of such a plastic substrate include a resin film and a resin thin film having a varnish effect, and such a plastic substrate is mainly used in flexible displays and foldable displays of mobile devices such as smartphones.

(Anode)

The material used in the anode is not particularly limited as long as the material can efficiently inject holes into the organic layer and is transparent or translucent in order to take out light. Examples of the material include electroconductive metal oxides such as zinc oxide, tin oxide, indium oxide, indium tin oxide (ITO), and indium zinc oxide (IZO), metals such as gold, silver, and chromium, inorganic electroconductive substances such as copper iodide and copper sulfide, and electroconductive polymers such as polythiophene, polypyrrole, and polyaniline, and ITO glass and NESA glass are particularly desirably used. These electrode materials may be used alone, or a plurality of the materials may be stacked or mixed for use.

(Cathode)

The material used in the cathode is not particularly limited as long as the material can efficiently inject electrons into the emissive layer. Preferable examples of the material generally include metals such as platinum, gold, silver, copper, iron, tin, aluminum, and indium, and alloys and multilayer laminated bodies of such a metal with a metal having a low work function such as lithium, sodium, potassium, calcium, or magnesium. Among these materials, aluminum, silver, and magnesium are preferable as a main component from the viewpoints of the electric resistance value, ease of film formation, the stability of a film, luminous efficiency, and the like. The cathode including magnesium and silver is particularly preferable because such a cathode facilitates electron injection into the electron transporting layer and the electron injection layer in the present invention to reduce the driving voltage.

(Protective Layer)

In order to protect the cathode, the cathode is preferably laminated with a protective layer (cap layer). The material included in the protective layer is not particularly limited, and examples of the material include metals such as platinum, gold, silver, copper, iron, tin, aluminum, and indium, alloys in which such a metal is used, inorganic substances such as silica, titania, and silicon nitride, and organic polymer compounds such as polyvinyl alcohol, polyvinyl chloride, and hydrocarbon-based polymer compounds. However, in a case where the light-emitting element has an element structure in which light is taken out from the cathode side (top emission structure), the material used in the protective layer is selected from materials having light permeability in a visible light region.

(Hole Injection Layer)

The hole injection layer is interposed between the anode and the hole transporting layer. The hole injection layer may be a single layer or a laminate in which a plurality of layers are stacked. The hole injection layer present between the hole transporting layer and the anode is preferable because with such a hole injection layer, the driving voltage is further reduced, the durable life is improved, and in addition, the carrier balance of the element is improved to improve the luminous efficiency.

The material used in the hole injection layer is not particularly limited, and examples of the material include benzidine derivatives, materials called starburst arylamines such as 4,4′,4″-tris(3-methylphenyl(phenyl)amino)triphenylamine (m-MTDATA) and 4,4′,4″-tris(1-naphthyl(phenyl)amino)triphenylamine (1-TNATA), heterocyclic compounds such as biscarbazole derivatives, pyrazoline derivatives, stilbene-based compounds, hydrazone-based compounds, benzofuran derivatives, thiophene derivatives, oxadiazole derivatives, phthalocyanine derivatives, and porphyrin derivatives, and polymer-based materials such as polycarbonates and styrene derivatives having the above-described monomer in a side chain, polythiophene, polyaniline, polyfluorene, polyvinylcarbazole, and polysilane. Among the materials, benzidine derivatives and starburst arylamine-based materials are more preferably used from the viewpoint of having a shallower HOMO level than the compound used in the hole transporting layer and smoothly injecting and transporting holes from the anode to the hole transporting layer.

These materials may be used alone, or two or more materials may be mixed and used. Furthermore, a plurality of the materials may be stacked to form a hole injection layer.

In the hole injection layer, an acceptor compound is preferably included alone, or a hole injection material as described above doped with an acceptor compound is preferably used, and as a result, the above-described effect can be obtained more remarkably. An acceptor compound forms a charge transfer complex with a hole transporting layer in contact with the acceptor compound when used as a single layer film, and forms a charge transfer complex with a material included in a hole injection layer when used for doping. Use of such a material leads to improvement in the electroconductivity of the hole injection layer, further contributes to reduction in the driving voltage of the element, and results in effects such as improvement in the luminous efficiency and improvement in the durable life.

Examples of the acceptor compound include metal chlorides such as iron(III) chloride, aluminum chloride, gallium chloride, indium chloride, and antimony chloride, metal oxides such as molybdenum oxide, vanadium oxide, tungsten oxide, and ruthenium oxide, and charge transfer complexes such as tris(4-bromophenyl)aminium hexachloroantimonate (TBPAH). In addition, organic compounds having a nitro group, a cyano group, a halogen, or a trifluoromethyl group in the molecule, such as 1,4,5,8,9,11-hexaazatriphenylene-hexacarbonitrile (HAT-CN6), 2,3,5,6-tetrafluoro-7,7,8,8-tetracyanoquinodimethane (F4-TCNQ), and fluorinated copper phthalocyanine, quinone-based compounds, acid anhydride-based compounds, fullerene, and the like are suitably used.

Among these compounds, metal oxides and cyano group-containing compounds are preferable. Because metal oxides and cyano group-containing compounds are easily handled and deposited, and therefore the above-described effects are easily obtained. In either of the case in which the hole injection layer includes an acceptor compound alone or the case in which the hole injection layer is doped with an acceptor compound, the hole injection layer may be a single layer or may include a plurality of layers that are stacked.

(Hole Transporting Layer)

The hole transporting layer is configured to transport holes injected from the anode to the emissive layer. The hole transporting layer may be a single layer or may include a plurality of layers that are stacked.

The hole transporting layer is formed with a method in which one or two or more hole transporting materials are stacked or mixed, or a method in which a mixture of a hole transporting material and a polymer binder is used. The hole transporting material is to transport holes from the anode efficiently between electrodes to which an electric field is applied, and preferably has high hole injection efficiency and efficiently transports injected holes. For this purpose, the hole transporting material is to be a substance that has an appropriate ionization potential, a high hole mobility, and excellent stability, and is less likely to generate an impurity as a trap when produced and used.

Examples of the substance satisfying such conditions include, and are not particularly limited to, benzidine derivatives, materials called starburst arylamines, heterocyclic compounds such as biscarbazole derivatives, pyrazoline derivatives, stilbene-based compounds, hydrazone-based compounds, benzofuran derivatives, thiophene derivatives, oxadiazole derivatives, phthalocyanine derivatives, and porphyrin derivatives, and polymer-based materials such as polycarbonates and styrene derivatives having the above-described monomer in a side chain, polythiophene, polyaniline, polyfluorene, polyvinylcarbazole, and polysilane.

(Emissive Layer)

The emissive layer may include a single material, but preferably includes a first compound and a second compound that is a dopant that exhibits strong light emission. Suitable examples of the first compound include a host material responsible for charge transfer and a thermally activated delayed fluorescent compound. The pyrromethene metal complex represented by the general formula (1) or (2) has a particularly excellent fluorescence quantum yield and an emission spectrum having a narrow half-value width, and therefore is preferably used as the second compound that is a dopant of the emissive layer.

If the doping amount with the second compound is too large, a concentration quenching phenomenon occurs. Therefore, the second compound is preferably used in an amount of 20% by weight or less, more preferably 10% by weight or less, and still more preferably 5% by weight or less with respect to the host material. If the doping concentration is too low, sufficient energy transfer rarely occurs. Therefore, the second compound is preferably used in an amount of 0.1% by weight or more, and more preferably 0.5% by weight or more with respect to the host material.

The emissive layer may include a compound, as an emissive material (host material or dopant material), other than the first compound and the second compound. Such a compound is referred to as another emissive material.

The host material is not necessarily to include only one compound, and a plurality of the compounds of the present invention may be mixed and used, or one or more other host materials may be mixed and used. Alternatively, the host materials may be stacked and used. The host material is not particularly limited. Examples of the host material that can be used include, but are not particularly limited to, compounds having a fused aryl ring and their derivatives, aromatic amine derivatives such as N,N′-dinaphthyl-N,N′-diphenyl-4,4′-diphenyl-1,1′-diamine, metal chelated oxinoid compounds such as tris(8-quinolinato)aluminum (III), bisstyryl derivatives such as distyrylbenzene derivatives, tetraphenylbutadiene derivatives, indene derivatives, coumarin derivatives, oxadiazole derivatives, pyrrolopyridine derivatives, perinone derivatives, cyclopentadiene derivatives, pyrrolopyrrole derivatives, thiadiazolopyridine derivatives, dibenzofuran derivatives, carbazole derivatives, indolocarbazole derivatives, triazine derivatives, and polymer-based materials such as polyphenylenevinylene derivatives, polyparaphenylene derivatives, polyfluorene derivatives, polyvinylcarbazole derivatives, and polythiophene derivatives.

Particularly preferable host materials are anthracene derivatives and naphthacene derivatives.

The dopant material may contain a compound other than the pyrromethene metal complex represented by the general formula (1) or (2). Such a compound is not particularly limited, and examples of the compound include compounds having a fused aryl ring and their derivatives, compounds having a heteroaryl ring and their derivatives, distyrylbenzene derivatives, aminostyryl derivatives, aromatic acetylene derivatives, tetraphenylbutadiene derivatives, stilbene derivatives, aldazine derivatives, pyrromethene derivatives, diketopyrrolo[3,4-c]pyrrole derivatives, coumarin derivatives, azole derivatives and their metal complexes, and aromatic amine derivatives. Among the compounds, dopants containing a diamine skeleton and dopants containing a fluoranthene skeleton are preferable because highly efficient light emission can be easily obtained with such a dopant. Dopants containing a diamine skeleton have a high hole trapping property, and dopants containing a fluoranthene skeleton have a high electron trapping property.

The emissive layer may include a phosphorescence emitting material. The phosphorescence emitting material emits phosphorescence even at room temperature. The dopant that emits phosphorescence is preferably a metal complex compound containing at least one metal selected from the group consisting of iridium (Ir), ruthenium (Ru), palladium (Pd), platinum (Pt), osmium (Os), and rhenium (Re). The ligand preferably has a nitrogen-containing aromatic heterocyclic ring such as a phenylpyridine skeleton, a phenylquinoline skeleton, or a carbene skeleton. However, the complex is not limited thereto, and an appropriate complex is selected considering the required emission color, element performance, and relationship with the host compound. An iridium complex or a platinum complex is preferably used because highly efficient light emission can be easily obtained.

However, from the viewpoint of enhancing the color purity, the dopant material is preferably one pyrromethene metal complex represented by the general formula (1) or (2).

The emissive layer may further include, other than the host material and the phosphorescence emitting material, a third component for adjustment of the carrier balance in the emissive layer or for stabilization of the layer structure of the emissive layer. However, as the third component, a material is selected that does not interact with the host material and the dopant material.

The thermally activated delayed fluorescence material is generally called a TADF material, and is a material in which the generation probability of a singlet exciton is improved by reducing the energy gap between the energy level of the singlet excited state and the energy level of the triplet excited state to promote inverse intersystem crossing from the triplet excited state to the singlet excited state. Fluorescence emission from the singlet exciton of the second compound is observed through Förster type energy transfer from the singlet exciton of the first compound having thermally activated delayed fluorescence to the singlet exciton of the second compound. By using the delayed fluorescence by the TADF mechanism, the theoretical internal efficiency can be enhanced to 100%. As described above, if the emissive layer includes the thermally activated delayed fluorescence material, further highly efficient light emission can be achieved to contribute to low power consumption of a display. In the thermally activated delayed fluorescence material, the thermally activated delayed fluorescence may be exhibited with a single material or a plurality of materials.

The thermally activated delayed fluorescent compound may include a single material or a plurality of materials, and known materials can be used. Specific examples of the material include benzonitrile derivatives, triazine derivatives, disulfoxide derivatives, carbazole derivatives, indolocarbazole derivatives, dihydrophenazine derivatives, thiazole derivatives, and oxadiazole derivatives. The compounds are particularly preferable that have an electron-donating moiety (donor moiety) and an electron withdrawing moiety (acceptor moiety) in the same molecule.

Here, examples of the electron-donating moiety (donor moiety) include aromatic amino groups and π-electron rich heterocyclic functional groups. Specific examples thereof include a diarylamino group, a carbazolyl group, a benzocarbazolyl group, a dibenzocarbazolyl group, an indolocarbazolyl group, a dihydroacridinyl group, a phenoxazinyl group, and a dihydrophenazinyl group. Examples of the electron withdrawing moiety (acceptor moiety) include phenyl groups having an electron withdrawing group as a substituent and n electron deficient heterocyclic functional groups. Specific examples thereof include phenyl groups having an electron withdrawing group selected from a carbonyl group, a sulfonyl group, and a cyano group as a substituent, and a triazinyl group. These functional groups may be substituted or unsubstituted.

Examples of such a thermally activated delayed fluorescent compound include, but are not particularly limited to, the following compounds.

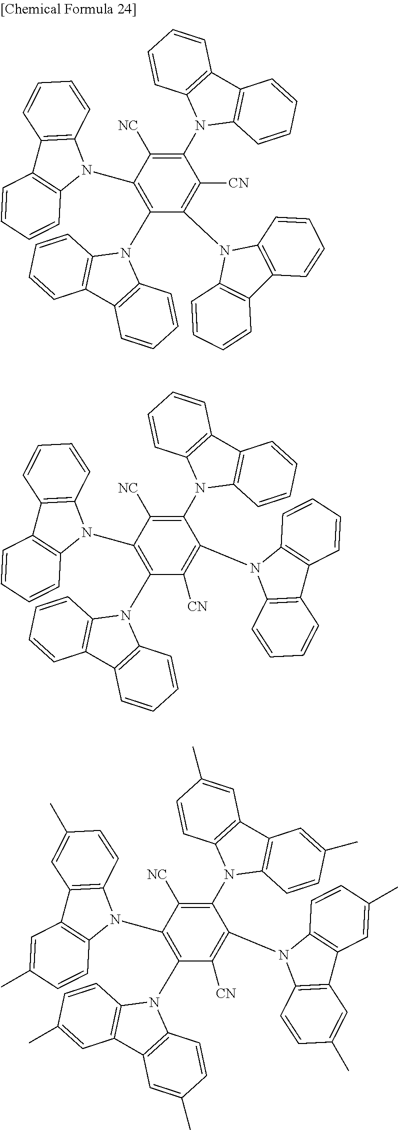

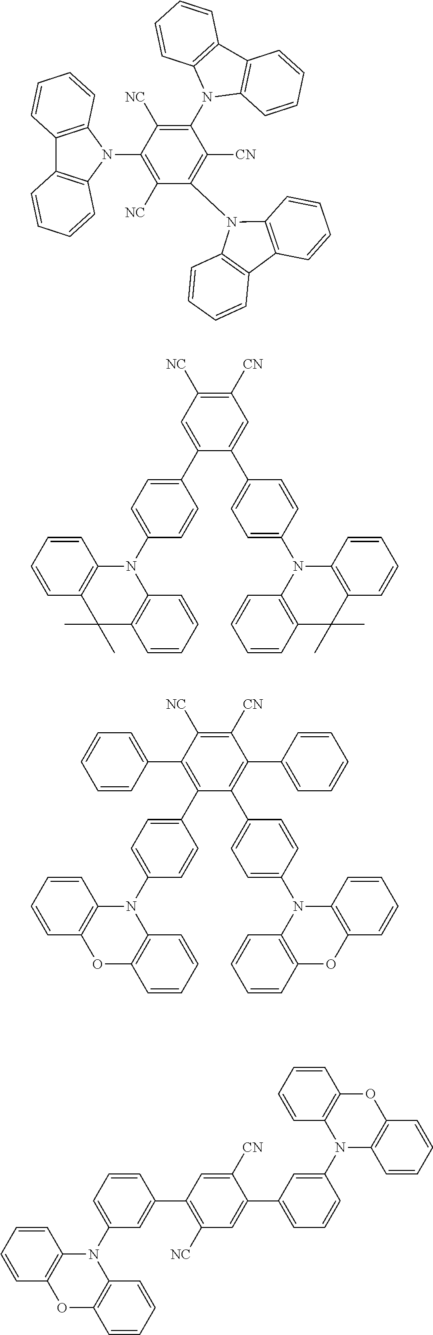 [SMILES: N#Cc1c(-n2c3ccccc3c3ccccc32)c(C#N)c(-n2c3ccccc3c3ccccc32)c(C#N)c1-n1c2ccccc2c2ccccc21 ; CC1(C)c2ccccc2N(c2ccc(-c3cc(C#N)c(C#N)cc3-c3ccc(N4c5ccccc5C(C)(C)c5ccccc54)cc3)cc2)c2ccccc21 ; N#Cc1c(C#N)c(-c2ccccc2)c(-c2ccc(N3c4ccccc4Oc4ccccc43)cc2)c(-c2ccc(N3c4ccccc4Oc4ccccc43)cc2)c1-c1ccccc1 ; N#Cc1cc(-c2cccc(N3c4ccccc4Oc4ccccc43)c2)c(C#N)cc1-c1cccc(N2c3ccccc3Oc3ccccc32)c1]

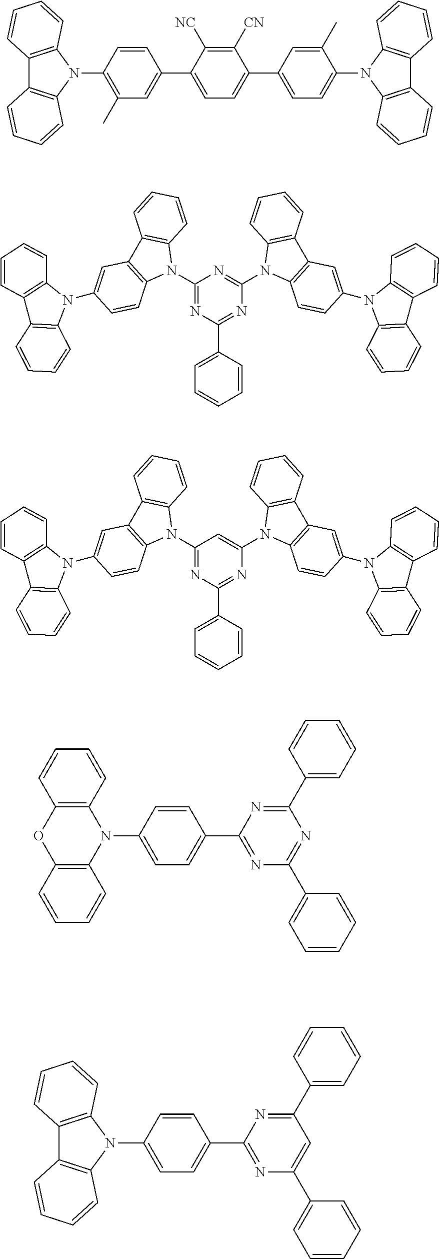 [SMILES: Cc1cc(-c2ccc(-c3ccc(-n4c5ccccc5c5ccccc54)c(C)c3)c(C#N)c2C#N)ccc1-n1c2ccccc2c2ccccc21 ; c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1 ; c1ccc(-c2nc(-n3c4ccccc4c4cc(-n5c6ccccc6c6ccccc65)ccc43)cc(-n3c4ccccc4c4cc(-n5c6ccccc6c6ccccc65)ccc43)n2)cc1 ; c1ccc(-c2nc(-c3ccccc3)nc(-c3ccc(N4c5ccccc5Oc5ccccc54)cc3)n2)cc1 ; c1ccc(-c2cc(-c3ccccc3)nc(-c3ccc(-n4c5ccccc5c5ccccc54)cc3)n2)cc1]

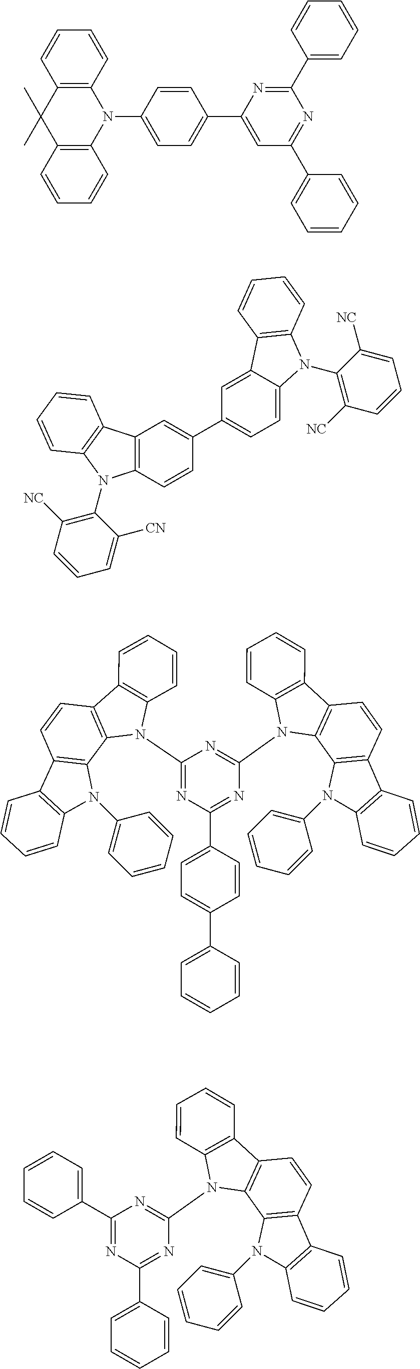 [SMILES: CC1(C)c2ccccc2N(c2ccc(-c3cc(-c4ccccc4)nc(-c4ccccc4)n3)cc2)c2ccccc21 ; N#Cc1cccc(C#N)c1-n1c2ccccc2c2cc(-c3ccc4c(c3)c3ccccc3n4-c3c(C#N)cccc3C#N)ccc21 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c6ccccc6n(-c6ccccc6)c5c43)n2)cc1]

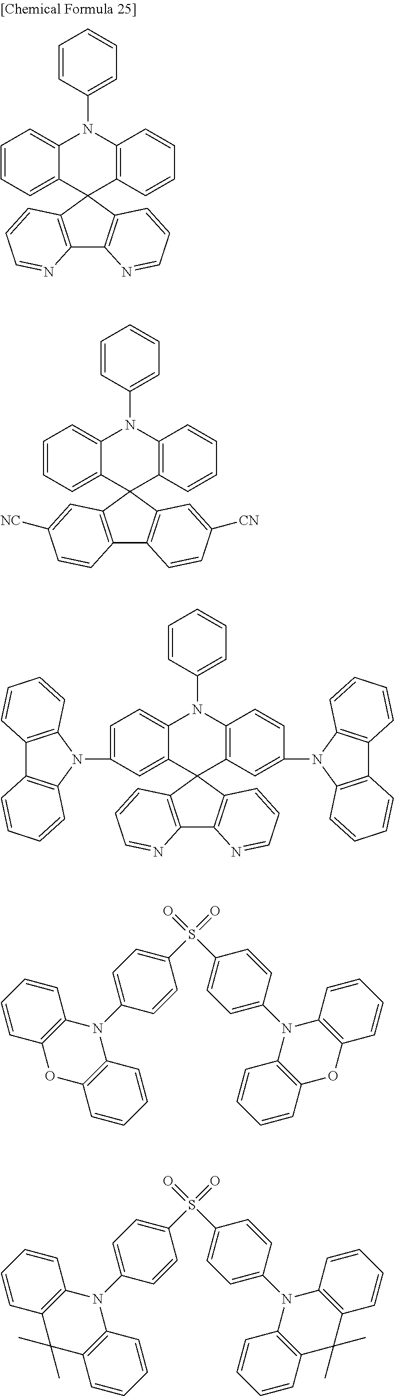 [SMILES: c1ccc(N2c3ccccc3C3(c4ccccc42)c2cccnc2-c2ncccc23)cc1 ; N#Cc1ccc2c(c1)C1(c3cc(C#N)ccc3-2)c2ccccc2N(c2ccccc2)c2ccccc21 ; c1ccc(N2c3ccc(-n4c5ccccc5c5ccccc54)cc3C3(c4cc(-n5c6ccccc6c6ccccc65)ccc42)c2cccnc2-c2ncccc23)cc1 ; O=S(=O)(c1ccc(N2c3ccccc3Oc3ccccc32)cc1)c1ccc(N2c3ccccc3Oc3ccccc32)cc1 ; CC1(C)c2ccccc2N(c2ccc(S(=O)(=O)c3ccc(N4c5ccccc5C(C)(C)c5ccccc54)cc3)cc2)c2ccccc21]

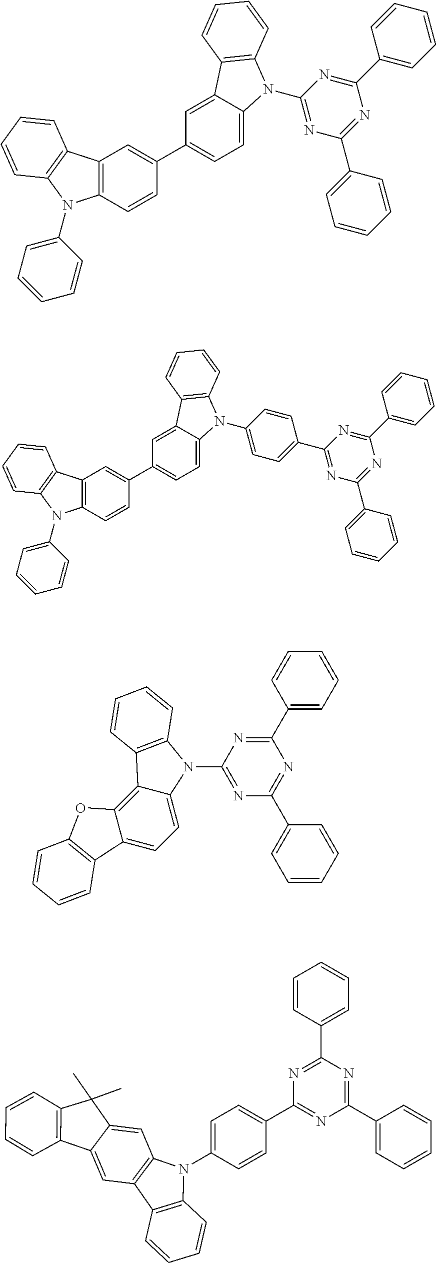 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-n3c4ccccc4c4c5oc6ccccc6c5ccc43)n2)cc1 ; CC1(C)c2ccccc2-c2cc3c4ccccc4n(-c4ccc(-c5nc(-c6ccccc6)nc(-c6ccccc6)n5)cc4)c3cc21]

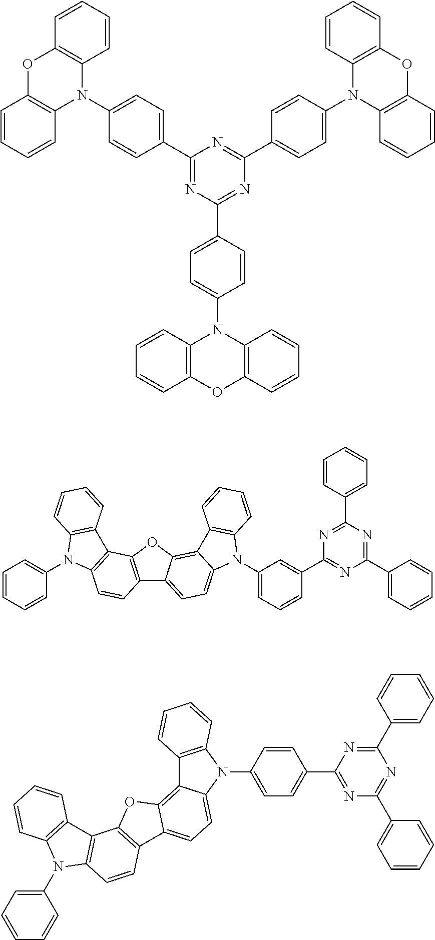 [SMILES: c1ccc2c(c1)Oc1ccccc1N2c1ccc(-c2nc(-c3ccc(N4c5ccccc5Oc5ccccc54)cc3)nc(-c3ccc(N4c5ccccc5Oc5ccccc54)cc3)n2)cc1 ; c1ccc(-c2nc(-c3ccccc3)nc(-c3cccc(-n4c5ccccc5c5c6oc7c(ccc8c7c7ccccc7n8-c7ccccc7)c6ccc54)c3)n2)cc1 ; c1ccc(-c2nc(-c3ccccc3)nc(-c3ccc(-n4c5ccccc5c5c6oc7c(ccc8c7c7ccccc7n8-c7ccccc7)c6ccc54)cc3)n2)cc1]

In the case that the thermally activated delayed fluorescence is exhibited with a plurality of materials, an electron transporting material (acceptor) and a hole transporting material (donor) are preferably combined to form an excited complex (exciplex). In the excited complex, the difference between the level of the singlet excited state and the level of the triplet excited state is so small that the energy transfers from the level of the triplet excited state to the level of the singlet excited state easily to improve the luminous efficiency. The efficiency of energy transfer can be enhanced through adjustment of the light emission wavelength of the excitation complex by adjusting the mixing ratio of the electron transporting material and the hole transporting material. Examples of such an electron transporting material include compounds containing a n electron deficient heteroaromatic ring and metal complexes. Specific examples include metal complexes such as bis(2-methyl-8-quinolinolato) (4-phenylphenolato)aluminum (III), bis(8-quinolinolato)zinc (II), and bis[2-(2-benzoxazolyl)phenolato]zinc (II), heterocyclic compounds having a polyazole skeleton such as 2-(4-biphenylyl)-5-(4-tert-butylphenyl)-1,3,4-oxadiazole, 3-(4-biphenylyl)-4-phenyl-5-(4-tert-butylphenyl)-1,2,4-triazole, and 2-[3-(dibenzothiophene-4-yl)phenyl]-1-phenyl-1H-benzimidazole, heterocyclic compounds having a diazine skeleton such as 2-[3′-(dibenzothiophene-4-yl)biphenyl-3-yl]dibenzo[f,h]quinoxaline, and 4,6-bis[3-(phenanthrene-9-yl)phenyl]pyrimidine, and heterocyclic compounds having a pyridine skeleton such as 3,5-bis[3-(9H-carbazole-9-yl)phenyl]pyridine. Meanwhile, examples of the hole transporting material include compounds containing a n electron rich heteroaromatic ring and aromatic amine compounds.

Specific examples include compounds having an aromatic amine skeleton such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenyl-9H-carbazole-3-yl)triphenylamine, 4-(1-naphthyl)-4′-(9-phenyl-9H-carbazole-3-yl)triphenylamine, and N-phenyl-N-[4-(9-phenyl-9H-carbazole-3-yl)phenyl]-spiro-9,9′-bifluorene-2-amine, compounds having a carbazole skeleton such as 1,3-bis(N-carbazolyl)benzene, 4,4′-di(N-carbazolyl)biphenyl (CBP), 3,3′-di(N-carbazolyl)biphenyl (mCBP), 1,3,5-tris[4-(N-carbazolyl)phenyl]benzene (TCPB), 9-[4-(10-phenyl-9-anthracenyl)phenyl]-9H-carbazole (CzPA), 1,4-bis[4-(N-carbazolyl)phenyl]-2,3,5,6-tetraphenylbenzene, 9-phenyl-9H-3-(9-phenyl-9H-carbazole-3-yl)carbazole, 3,6-bis[N-(9-phenylcarbazole-3-yl)-N-phenylamino]-9-phenylcarbazole (PCzPCA2), 3-[N-(1-naphthyl)-N-(9-phenylcarbazole-3-yl)amino]-9-phenylcarbazole (PCzPCN1), 9-([1,1-biphenyl]-4-yl)-9′-([1,1′:4′,1″-terphenyl]-4-yl)-9H,9′H-3,3′-bicarbazole, 9-([1,1′:4′,1″-terphenyl]-4-yl)-9′-(naphthalene-2-yl)-9H,9′H-3,3′-bicarbazole, and 9,9′,9″-triphenyl-9H,9′H,9″H-3,3′:6′,3″-tricarbazole, compounds having a thiophene skeleton such as 4,4′,4″-(benzene-1,3,5-triyl)tri(dibenzothiophene) and 2,8-diphenyl-4-[4-(9-phenyl-9H-fluorene-9-yl)phenyl]dibenzothiophene, and compounds having a furan skeleton such as 4,4′,4″-(benzene-1,3,5-triyl)tri(dibenzofuran), and 4-{3-[3-(9-phenyl-9H-fluorene-9-yl)phenyl]phenyl}dibenzofuran.

If the first compound is a thermally activated delayed fluorescent compound and if a compound other than the first compound and the second compound, that is, another emissive material is further included, the emissive material (host material or dopant material) is referred to as a third compound. In other words, if the emissive layer includes a third compound, the first compound is a thermally activated delayed fluorescent compound.

It is preferable that the first compound be a thermally activated delayed fluorescent compound, the emissive layer further include a third compound, and the singlet excitation energy of the third compound be larger than the singlet excitation energy of the first compound. It is more preferable that the triplet excitation energy of the third compound be larger than the triplet excitation energy of the first compound. As a result, the third compound can have a function of confining the energy of the emissive material in the emissive layer, and efficient light emission can be achieved.

The third compound is to have a function, for example, as a host material, and is preferably an organic compound that has high charge transporting ability and a high glass transition temperature. Examples of the third compound include, but are not particularly limited to, the following compounds.

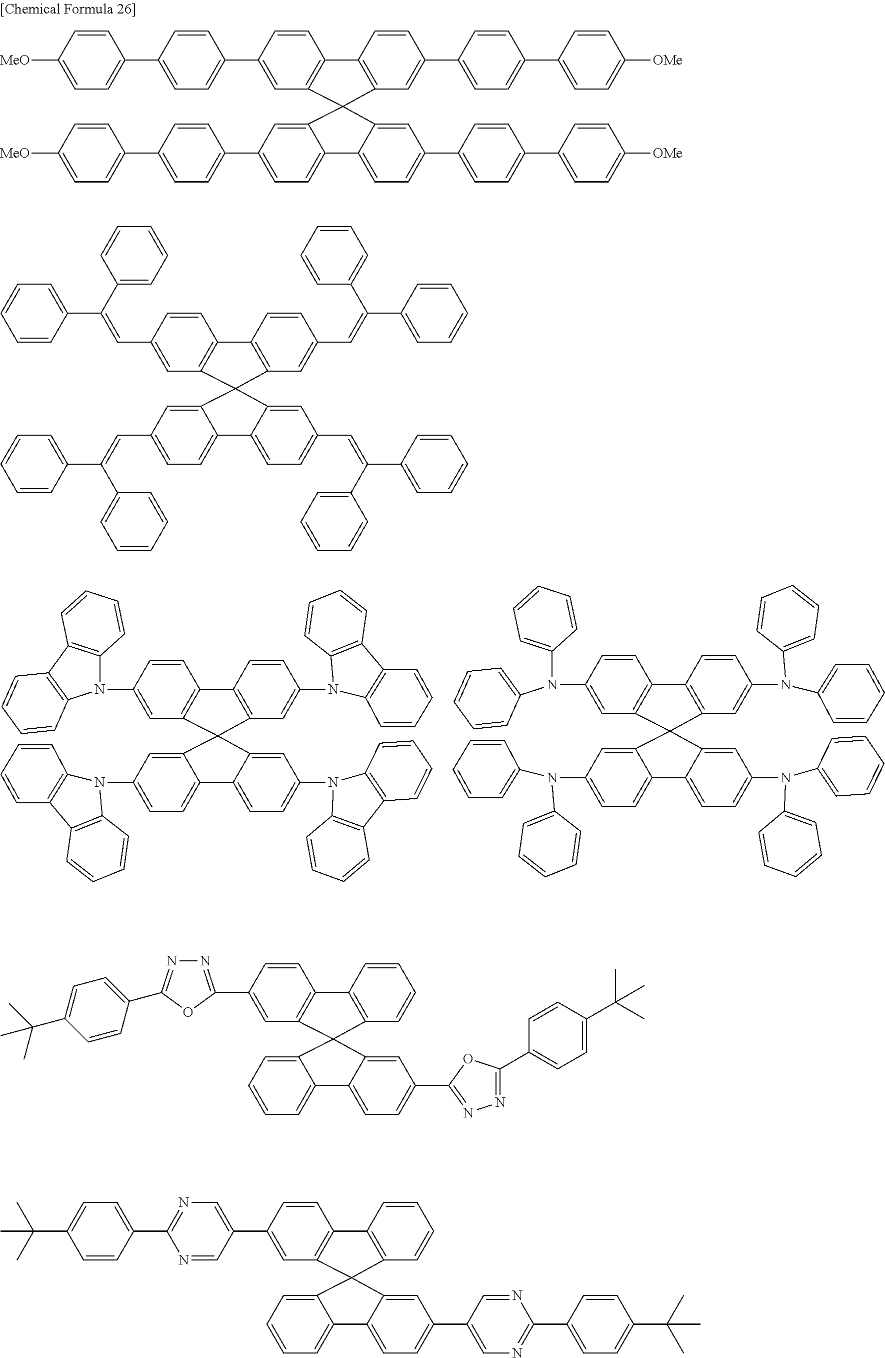

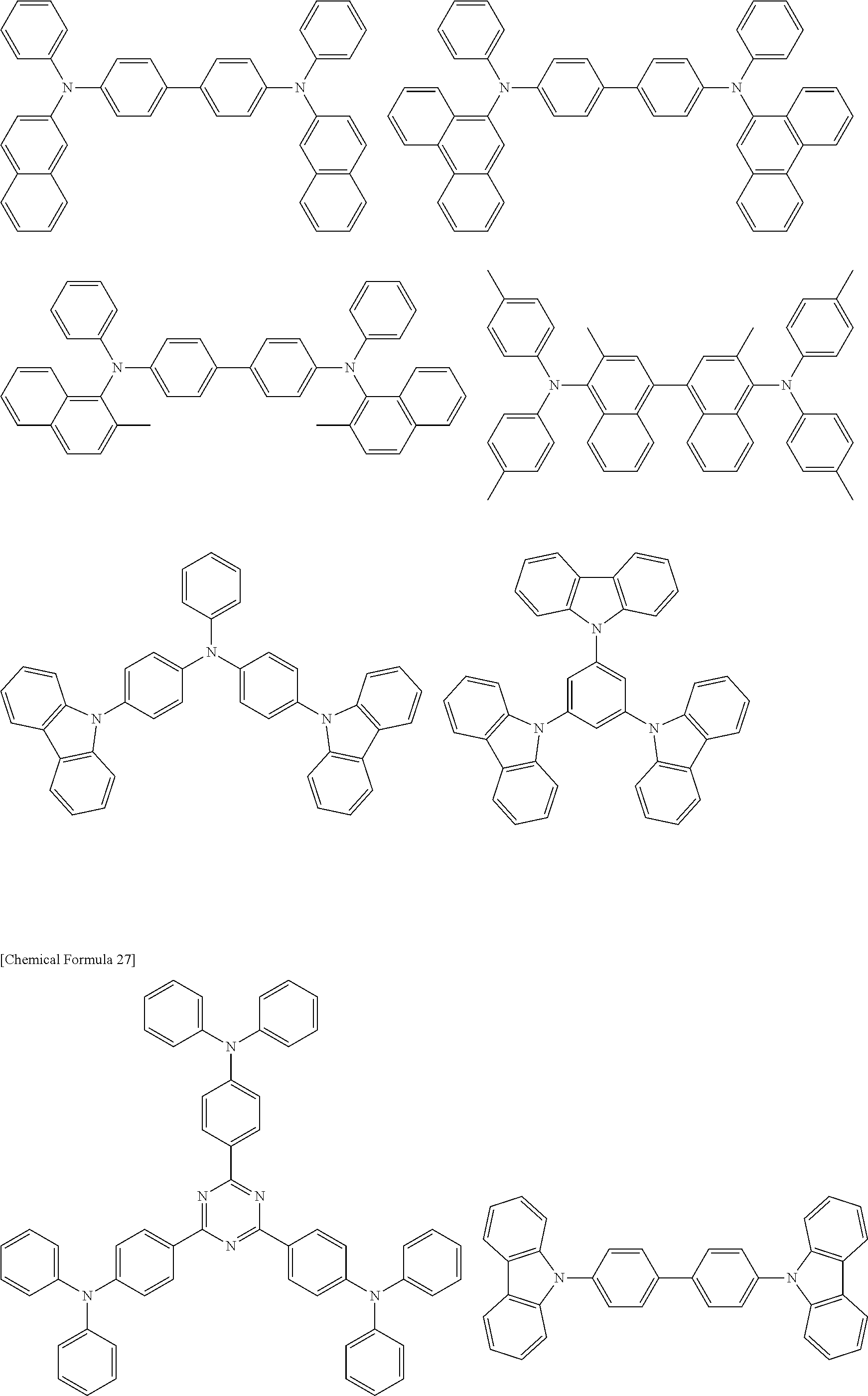

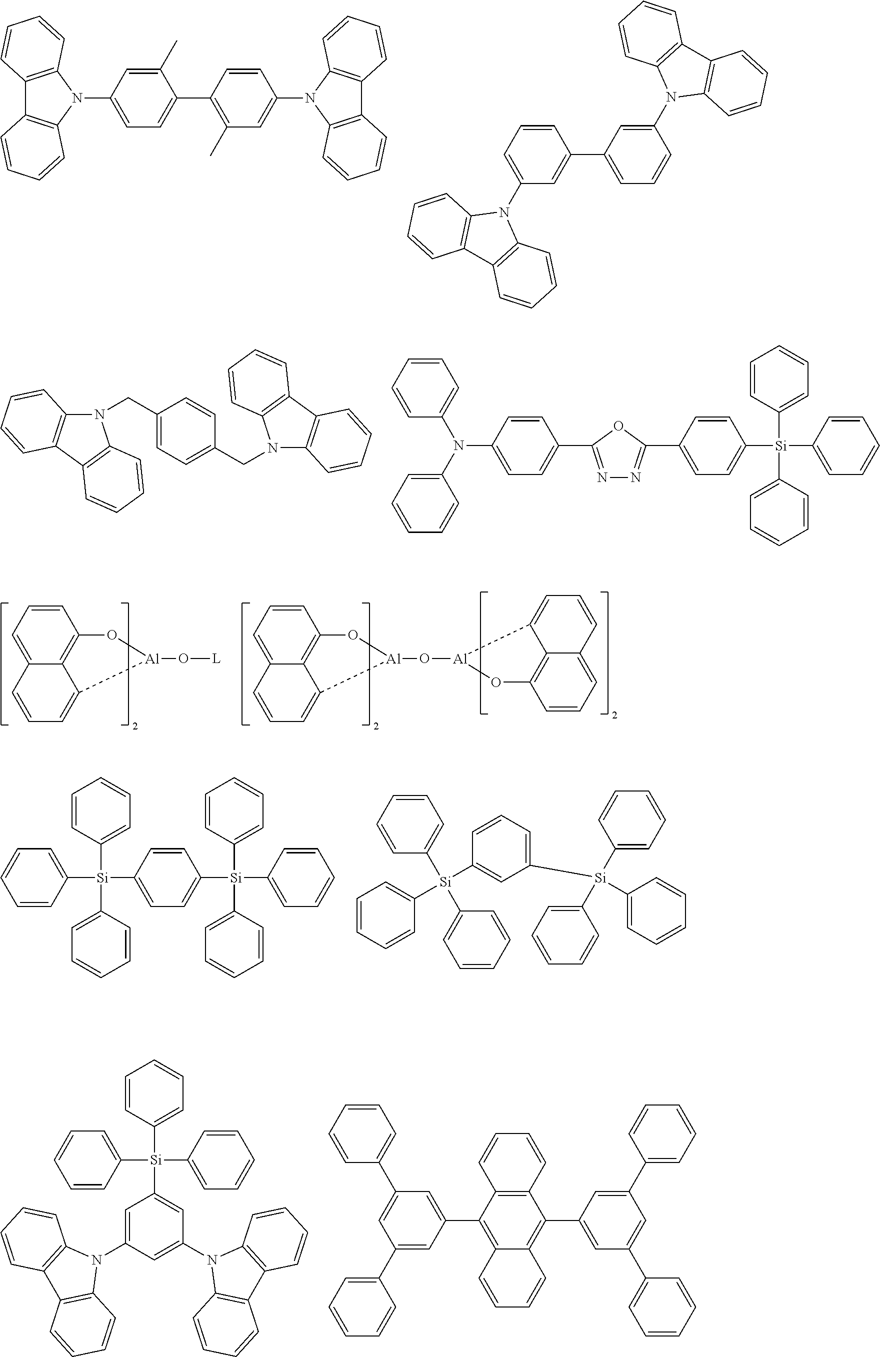

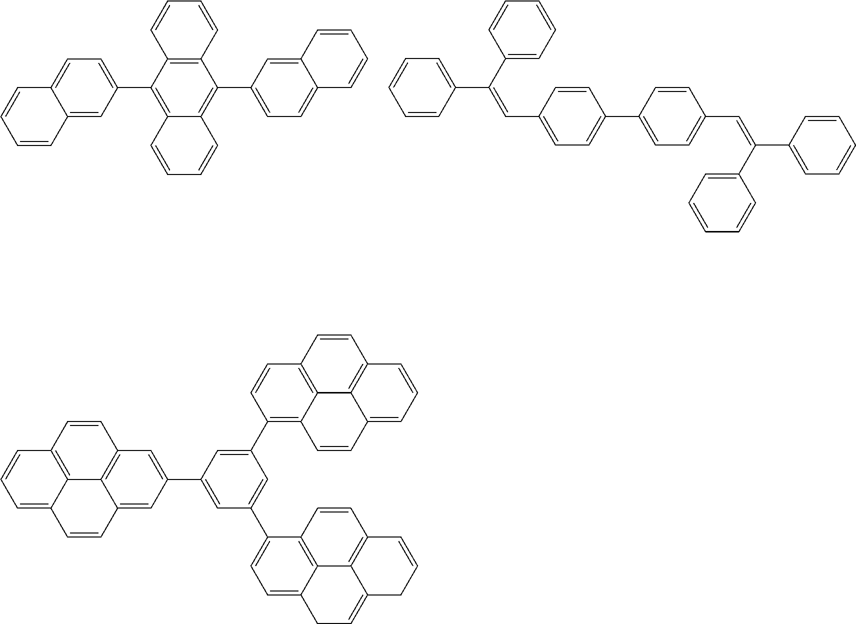

The third compound may include a single material or a plurality of materials. The third compound preferably includes two or more materials. In the case that a plurality of materials are used as the third compound, the third compound is preferably a combination of an electron transporting third compound and a hole transporting third compound. Through combination of an electron transporting third compound and a hole transporting third compound at an appropriate mixing ratio, the charge balance in the emissive layer is adjusted to suppress the deviation of the light emitting region, and thus the reliability and the durability of the light-emitting element can be improved. An excited complex may be formed between the electron transporting third compound and the hole transporting third compound. From the above viewpoint, it is preferable to satisfy each relational expression of the formulae 1 to 4. It is more preferable to satisfy the formulae 1 and 2, and it is still more preferable to satisfy the formulae 3 and 4. It is still even more preferable to satisfy all of the formulae 1 to 4.

S₁(electron transporting third compound)>S₁(first compound) (the formula 1)

S₁(hole transporting third compound)>S₁(first compound) (the formula 2)

T₁(electron transporting third compound)>T₁(first compound) (the formula 3)

T₁(hole transporting third compound)>T₁(first compound) (the formula 4)

Here, S₁ represents an energy level of the singlet excited state of each compound, and T₁ represents an energy level of the triplet excited state of each compound.

Examples of the electron transporting third compound include compounds containing a n electron deficient heteroaromatic ring. Specific examples include heterocyclic compounds having a polyazole skeleton such as 2-(4-biphenylyl)-5-(4-tert-butylphenyl)-1,3,4-oxadiazole (PBD), 3-(4-biphenylyl)-4-phenyl-5-(4-tert-butylphenyl)-1,2,4-triazole (TAZ), 1,3-bis[5-(p-tert-butylphenyl)-1,3,4-oxadiazole-2-yl]benzene (OXD-7), 9-[4-(5-phenyl-1,3,4-oxadiazole-2-yl)phenyl]-9H-carbazole (CO11), 2,2′,2″-(1,3,5-benzenetriyl)tris(1-phenyl-1H-benzimidazole) (TPBI), and 2-[3-(dibenzothiophene-4-yl)phenyl]-1-phenyl-1H-benzimidazole (mDBTBIm-II), heterocyclic compounds having a quinoxaline skeleton or a dibenzoquinoxaline skeleton such as 2-[3-(dibenzothiophene-4-yl)phenyl]dibenzo[f,h]quinoxaline (2mDBTPDBq-II), 2-[3′-(dibenzothiophene-4-yl)biphenyl-3-yl]dibenzo[f,h]quinoxaline (2mDBTBPDBq-II), 2-[4-(3,6-diphenyl-9H-carbazole-9-yl)phenyl]dibenzo[f,h]quinoxaline (2CzPDBq-III), 7-[3-(dibenzothiophene-4-yl)phenyl]dibenzo[f,h]quinoxaline (7mDBTPDBq-II), 6-[3-(dibenzothiophene-4-yl)phenyl]dibenzo[f,h]quinoxaline (6mDBTPDBq-II), and 2-[3′-(9H-carbazole-9-yl)biphenyl-3-yl]dibenzo[f,h]quinoxaline (2mCzBPDBq), heterocyclic compounds having a diazine skeleton (pyrimidine skeleton or pyrazine skeleton) such as 4,6-bis[3-(phenanthrene-9-yl)phenyl]pyrimidine (4,6mPnP2Pm), 4,6-bis[3-(9H-carbazole-9-yl)phenyl]pyrimidine (4,6mCzP2Pm), and 4,6-bis[3-(4-dibenzothienyl)phenyl]pyrimidine (4,6mDBTP2Pm-II), and heterocyclic compounds having a pyridine skeleton such as 3,5-bis[3-(9H-carbazol-9-yl)phenyl]pyridine (3,5DCzPPy), 1,3,5-tri[3-(3-pyridyl)phenyl]benzene (TmPyPB), and 3,3′,5,5′-tetra[(m-pyridyl)-phene-3-yl]biphenyl (BP4mPy).

Examples of the hole transporting third compound include compounds containing a n electron rich heteroaromatic ring. Specific examples include compounds having a carbazole skeleton such as 1,3-bis(N-carbazolyl)benzene, 4,4′-di(N-carbazolyl)biphenyl (CBP), 3,3′-di(N-carbazolyl)biphenyl (mCBP), 1,3,5-tris[4-(N-carbazolyl)phenyl]benzene (TCPB), 9-[4-(10-phenyl-9-anthracenyl)phenyl]-9H-carbazole (CzPA), 1,4-bis[4-(N-carbazolyl)phenyl]-2,3,5,6-tetraphenylbenzene, 9-phenyl-9H-3-(9-phenyl-9H-carbazole-3-yl)carbazole, 3,6-bis[N-(9-phenylcarbazole-3-yl)-N-phenylamino]-9-phenylcarbazole (PCzPCA2), 3-[N-(1-naphthyl)-N-(9-phenylcarbazole-3-yl)amino]-9-phenylcarbazole (PCzPCN1), 9-([1,1-biphenyl]-4-yl)-9′-([1,1′:4′,1″-terphenyl]-4-yl)-9H,9′H-3,3′-bicarbazole, 9-([1,1′:4′,1″-terphenyl]-4-yl)-9′-(naphthalene-2-yl)-9H,9′H-3,3′-bicarbazole, and 9,9′,9″-triphenyl-9H,9′H,9″H-3,3′:6′,3″-tricarbazole.

(Electron Transporting Layer)

In embodiments of the present invention, the electron transporting layer is a layer into which electrons are injected from the cathode and in which the electrons are transported. The electron transporting layer is desired to have high electron injection efficiency and to efficiently transport injected electrons. Therefore, the material used in the electron transporting layer is to be a substance that has a high electron affinity, a high electron mobility, and excellent stability, and is less likely to generate an impurity as a trap when produced and used. In particular, in the case of laminating a film having a large thickness, the film quality of a compound having a low molecular weight easily deteriorates due to crystallization or the like, and therefore a compound is preferable that has a molecular weight of 400 or more so that the film quality is stably maintained.

The term “electron transporting layer” in the present invention also means a hole inhibition layer capable of efficiently inhibiting the transfer of holes, and the hole inhibition layer and the electron transporting layer may include one material alone or a plurality of materials that are stacked.

Examples of the electron transporting material used in the electron transporting layer include fused polycyclic aromatic derivatives, styryl-based aromatic ring derivatives, quinone derivatives, phosphorus oxide derivatives, and various metal complexes such as quinolinol complexes, for example, tris(8-quinolinolato)aluminum(III), benzoquinolinol complexes, hydroxyazole complexes, azomethine complexes, tropolone metal complexes, and flavonol metal complexes. It is preferable to use a compound that includes an element selected from carbon, hydrogen, nitrogen, oxygen, silicon, and phosphorus and has a heteroaryl ring structure including electron-accepting nitrogen, because the driving voltage is reduced and highly efficient light emission can be obtained with such a compound.

Here, the term “electron-accepting nitrogen” refers to a nitrogen atom forming a multiple bond with an adjacent atom. A nitrogen atom has a high electron negativity, and therefore the multiple bond has an electron-accepting property. Therefore, an aromatic heterocyclic ring including electron-accepting nitrogen has high electron affinity. An electron transporting material having electron-accepting nitrogen facilitates reception of electrons from a cathode having a high electron affinity to further reduce the driving voltage. Furthermore, electrons supplied to the emissive layer is increased to enhance the recombination probability, and as a result, the luminous efficiency is improved.

Examples of the heteroaryl ring including electron-accepting nitrogen include a triazine ring, a pyridine ring, a pyrazine ring, a pyrimidine ring, a quinoline ring, a quinoxaline ring, a quinazoline ring, a naphthyridine ring, a pyrimidopyrimidine ring, a benzoquinoline ring, a phenanthroline ring, an imidazole ring, an oxazole ring, an oxadiazole ring, a triazole ring, a triazole ring, a thiadiazole ring, a benzoxazole ring, a benzothiazole ring, a benzimidazole ring, and a phenanthroimidazole ring.

Examples of the preferable compound having such a heteroaryl ring structure include pyridine derivatives, triazine derivatives, quinazoline derivatives, pyrimidine derivatives, benzimidazole derivatives, benzoxazole derivatives, benzthiazole derivatives, oxadiazole derivatives, thiadiazole derivatives, triazole derivatives, pyrazine derivatives, phenanthroline derivatives, quinoxaline derivatives, quinoline derivatives, benzoquinoline derivatives, oligopyridine derivatives such as bipyridine and terpyridine, quinoxaline derivatives, and naphthyridine derivatives. Among the derivatives, imidazole derivatives such as tris(N-phenylbenzimidazol-2-yl)benzene, oxadiazole derivatives such as 1,3-bis[(4-tert-butylphenyl)-1,3,4-oxadiazolyl]phenylene, triazole derivatives such as N-naphthyl-2,5-diphenyl-1,3,4-triazole, phenanthroline derivatives such as bathocuproine and 1,3-bis(1,10-phenanthroline-9-yl)benzene, benzoquinoline derivatives such as 2,2′-bis(benzo[h]quinolin-2-yl)-9,9′-spirobifluorene, bipyridine derivatives such as 2,5-bis(6′-(2′,2″-bipyridyl))-1,1-dimethyl-3,4-diphenylsilole, terpyridine derivatives such as 1,3-bis(4′-(2,2′:6′2″-terpyridinyl))benzene, and naphthyridine derivatives such as bis(1-naphthyl)-4-(1,8-naphthyridine-2-yl)phenylphosphine oxide are preferably used from the viewpoint of electron transporting ability.

It is preferable that these derivatives have a fused polycyclic aromatic skeleton because with such a derivative, the glass transition temperature is improved and the electron mobility is increased to obtain a large effect of reducing the voltage of the light-emitting element. Furthermore, considering improvement in the element durable life, ease of synthesis, and availability of the raw material, the fused polycyclic aromatic skeleton is more preferably a fluoranthene skeleton, an anthracene skeleton, a pyrene skeleton, or a phenanthroline skeleton, and particularly preferably a fluoranthene skeleton or a phenanthroline skeleton.

The electron transporting material is used singly, but two or more kinds of the electron transporting materials may be used in combination. The electron transporting layer may include a donor material. Here, the term “donor material” refers to a compound that facilitates electron injection from the cathode or the electron injection layer into the electron transporting layer through improvement in the electron injection barrier, and improves the electroconductivity of the electron transporting layer.

Preferable examples of the donor material include alkali metals, inorganic salts containing an alkali metal, complexes of an alkali metal and an organic substance, alkaline earth metals, complexes of an inorganic salt containing an alkaline earth metal or an alkaline earth metal and an organic substance, rare earth metals such as Eu and Yb, inorganic salts containing a rare earth metal, and complexes of a rare earth metal and an organic substance. As the donor material, metallic lithium, rare earth metals, lithium fluoride, and lithium quinolinol (Liq) are particularly preferable.

(Electron Injection Layer)

In the present invention, an electron injection layer may be provided between the cathode and the electron transporting layer. The electron injection layer is interposed generally for the purpose of assisting injection of electrons from the cathode to the electron transporting layer. When the electron injection layer is interposed, a compound having a heteroaryl ring structure including electron-accepting nitrogen may be used, or a layer including the above-described donor material may be used.

In the electron injection layer, an inorganic substance as an insulator or a semiconductor can also be used. It is preferable to use these materials because by using these materials, a short circuit of the light-emitting element can be prevented and the electron injection property can be improved.

It is preferable to use, as such an insulator, at least one metal compound selected from the group consisting of alkali metal chalcogenides, alkaline earth metal chalcogenides, halides of an alkali metal, and halides of an alkaline earth metal.

(Charge Generation Layer)

The charge generation layer in the present invention may be formed into one layer or a laminate in which a plurality of layers are stacked. In general, a layer that easily generates an electron as a charge is called an n-type charge generation layer, and a layer that easily generates a hole is called a p-type charge generation layer. The charge generation layer preferably includes a double layer. Specifically, the charge generation layer is preferably used as a pn junction charge generation layer including an n-type charge generation layer and a p-type charge generation layer. The pn junction charge generation layer generates a charge or separates a charge into a hole and an electron when a voltage is applied in the light-emitting element, and the hole and the electron are injected into the emissive layer via the hole transporting layer and the electron transporting layer. Specifically, in the light-emitting element in which the emissive layers are stacked, the pn junction charge generation layer functions as a charge generation layer that is an intermediate layer. The n-type charge generation layer supplies electrons to the first emissive layer present on the anode side, and the p-type charge generation layer supplies holes to the second emissive layer present on the cathode side. Therefore, in the light-emitting element in which the plurality of emissive layers are stacked, the luminous efficiency can be improved, the driving voltage can be reduced, and the durability of the element is also improved.

The n-type charge generation layer includes an n-type dopant and an n-type host, and as the n-type dopant and the n-type host, conventional materials can be used. For example, as the n-type dopant, the above-described donor materials are suitably used, and specifically, alkali metals and their salts, alkaline earth metals and their salts, and rare earth metals can be used. Among the materials, alkali metals and their salts, and rare earth metals are preferable, and metallic lithium, lithium fluoride (LiF), lithium quinolinol (Liq), and metal ytterbium are more preferable. As the n-type host, the above-described electron transporting materials used in the electron transporting layer are suitably used, and among the materials, triazine derivatives, phenanthroline derivatives, and oligopyridine derivatives can be used. As the n-type host, the above-described electron transporting materials used in the electron transporting layer are suitably used. Among the materials, phenanthroline derivatives and terpyridine derivatives are preferable. The phenanthroline derivative represented by the general formula (13) is more preferable. That is, in the light-emitting element of the present invention, the charge generation layer preferably includes the phenanthroline derivative represented by the general formula (13). The phenanthroline derivative represented by the general formula (13) is preferably included in the n-type charge generation layer.

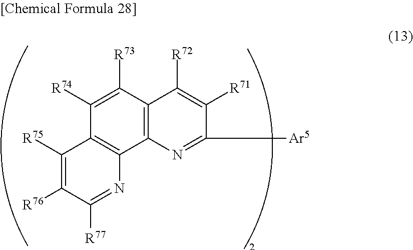

In the formula, Ar⁵ represents an aryl group substituted with two phenanthrolyl groups. The substitution positions are arbitrary. The aryl group may have another substituent at another position. Such an aryl group is preferably selected from a phenyl group, a naphthyl group, a phenanthryl group, a pyrenyl group, and a fluorenyl group from the viewpoints of ease of synthesis and sublimability.

R⁷¹ to R⁷⁷ are the same or different from each other, and are each selected from a hydrogen atom, an alkyl group, a cycloalkyl group, a heterocyclic group, an aryl group, and a heteroaryl group. In particular, from the viewpoints of stability and ease of charge transfer in the compound, R⁷¹ to R⁷⁷ are each preferably selected from a hydrogen atom, an alkyl group, an aryl group, and a heteroaryl group.

Examples of the phenanthroline derivative represented by the general formula (13) include the following compounds.

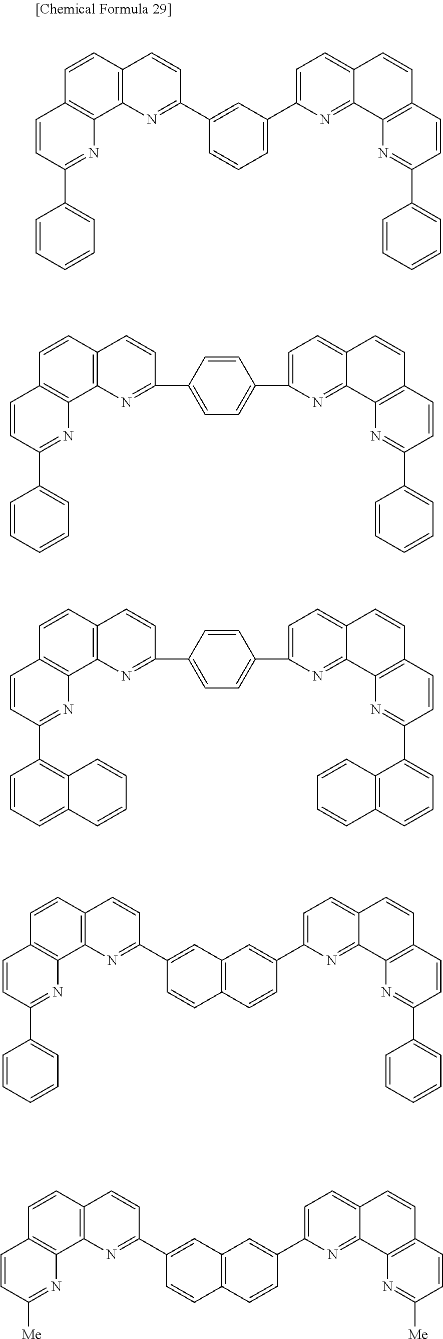

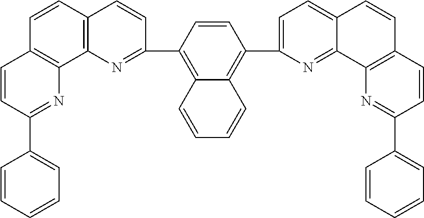

The p-type charge generation layer includes a p-type dopant and a p-type host, and as the p-type dopant and the p-type host, conventional materials can be used. For example, as the p-type dopant, the above-described acceptor compounds used in the hole injection layer are suitably used, and specifically, 1,4,5,8,9,11-hexaazatriphenylene-hexacarbonitrile (HAT-CN6), tetrafluore-7,7,8,8-tetracyanoquinodimethane (F4-TCNQ), tetracyanoquinodimethane derivatives, radialene derivatives, iodine, FeCl₃, FeF₃, SbCl₅, and the like can be used. Particularly preferable compounds are 1,4,5,8,9,11-hexaazatriphenylene-hexacarbonitrile (HAT-CN6) and radialene derivatives such as (2E,2′E,2″E)-2,2′,2″-(cyclopropane-1,2,3-triylidene)tris(2-(perfluorophenyl)-acetonitrile) and (2E,2′E,2″E)-2,2′,2″-(cyclopropane-1,2,3-triylidene)tris(2-(4-cyanoperfluorophenyl)-acetonitrile). The acceptor compound may singly form a thin film. In this case, the thin film of the acceptor compound more preferably has a thickness of 10 nm or less. The p-type host is preferably an arylamine derivative.

The method of forming each of the layers included in the light-emitting element may be a dry process or a wet process. Examples of the method include, and are not particularly limited to, resistance heating deposition, electron beam deposition, sputtering, a molecular lamination method, a coating method, an inkjet method, and a printing method, and from the viewpoint of an element characteristic, resistance heating deposition and electron beam deposition are usually preferable.

The thickness of the organic layer depends on the resistance value of the emissive substance and is not limited, but is preferably 1 to 1,000 nm. The emissive layer, the electron transporting layer, and the hole transporting layer each preferably have a film thickness of 1 nm or more and 200 nm or less, and more preferably 5 nm or more and 100 nm or less.

The light-emitting element according to an embodiment of the present invention has a function of converting electrical energy into light. Here, as the electrical energy, a direct current is mainly used, but a pulse current and an alternating current can also be used. The current value and the voltage value are not particularly limited, but in consideration of the power consumption and the life of the element, it is preferable to obtain the maximum luminance with as low energy as possible.

The light-emitting element according to an embodiment of the present invention preferably emits red light having a peak wavelength of 580 nm or more and 750 nm or less when energized. The peak wavelength is preferably in the region of 600 nm or more and 640 nm or less, and more preferably in the region of 600 nm or more and 630 nm or less from the viewpoint of expanding the color gamut to improve the color reproducibility.

The light-emitting element according to an embodiment of the present invention preferably has an emission spectrum having a half-value width of 45 nm or less, and more preferably 40 nm or less when energized from the viewpoint of enhancing the color purity.

The light-emitting element according to an embodiment of the present invention is suitably used, for example, as a display device such as a display that displays in a matrix and/or segment system.

The light-emitting element according to an embodiment of the present invention is also preferably used as a backlight for various devices and the like. The backlight is mainly used for the purpose of improving the visibility of a display device such as a display that does not emit light, and is used in display devices such as liquid crystal displays, watches, audio devices, automobile panels, display boards, and marks. In particular, the light-emitting element of the present invention is preferably used in a backlight for a liquid crystal display, particularly for a personal computer that is studied for reduction in the thickness, and a backlight thinner and lighter than conventional ones can be provided.

The light-emitting element according to an embodiment of the present invention is also preferably used as various illumination devices. The light-emitting element according to an embodiment of the present invention can achieve both high luminous efficiency and high color purity, and further can achieve reduction in the thickness and the weight, and therefore an illumination device can be realized that has low power consumption, a bright emitted color, and a high design property with the light-emitting element.

### EXAMPLES

Hereinafter, the present invention will be described with reference to Examples, but the present invention is not limited to Examples described below.

### Synthesis Example 1

Method of Synthesizing Compound D-1

A mixed solution of 4.50 g of 3-(4-tert-butylphenyl)-1,4,5,6-tetrahydrobenzo[6,7]cyclohepta[1,2-b]pyrrole, 3.25 g of 1-naphthoyl chloride, and 70 ml of o-xylene was heated and stirred at 130° C. for 5 hours under a nitrogen stream. After cooling to room temperature, methanol was added, and the precipitated solid was filtered out and vacuum-dried to obtain 5.60 g of 2-(1-naphthoyl)-3-(4-tert-butylphenyl)-1,4,5,6-tetrahydrobenzo[6,7]cyclohepta[1,2-b]pyrrole.

Next, a mixed solution of 1.35 g of 2-(1-naphthoyl)-3-(4-tert-butylphenyl)-1,4,5,6-tetrahydrobenzo[6,7]cyclohepta[1,2-b]pyrrole, 0.95 g of 3-(4-tert-butylphenyl)-1,4,5,6-tetrahydrobenzo[6,7]cyclohepta[1,2-b]pyrrole, 1.63 g of trifluoromethanesulfonic anhydride, and 30 ml of toluene was heated and stirred at 110° C. for 6 hours under a nitrogen stream. After cooling to room temperature, 50 ml of water was poured, and the resulting mixture was subjected to extraction with 50 ml of ethyl acetate. The organic layer was washed with 50 ml of water, then magnesium sulfate was added, and the resulting mixture was filtered. The solvent was removed from the filtrate with an evaporator to obtain a pyrromethene compound as a residue.

Next, to a mixed solution of the obtained pyrromethene compound and 60 ml of toluene, 3.0 ml of diisopropylethylamine and 2.2 ml of boron trifluoride diethyl ether complex were added under a nitrogen stream, and the resulting mixture was stirred at 80° C. for 1 hour. Next, 50 ml of water was poured, and the resulting mixture was subjected to extraction with 50 ml of ethyl acetate. The organic layer was washed with 50 ml of water, then magnesium sulfate was added, and the resulting mixture was filtered. The solvent was removed from the filtrate with an evaporator, and then the residue was purified with silica gel column chromatography (heptane/toluene=1/2). Furthermore, to the concentrated purified product, 50 ml of methanol was added, the resulting mixture was heated and stirred at 60° C. for 10 minutes and then cooled, and the precipitated solid was filtered out and vacuum-dried to obtain 1.56 g of a reddish purple powder. The obtained powder was analyzed with liquid chromatography-mass spectrometry (LC-MS), and it was confirmed that the reddish purple powder was a compound D-1 as a pyrromethene metal complex.

MS (m/z) 815 [M+H]⁺ Compound D-1:

The compound D-1 was subjected to sublimation purification using an oil diffusion pump under a pressure of 1×10⁻³ Pa at 270° C. and then used as a light-emitting element material.

The light emission characteristics in the solution of the compound D-1 are shown below.

Absorption spectrum (solvent: toluene): λmax 584 nm

Fluorescence spectrum (solvent: toluene): λmax 607 nm, half-value width 35 nm

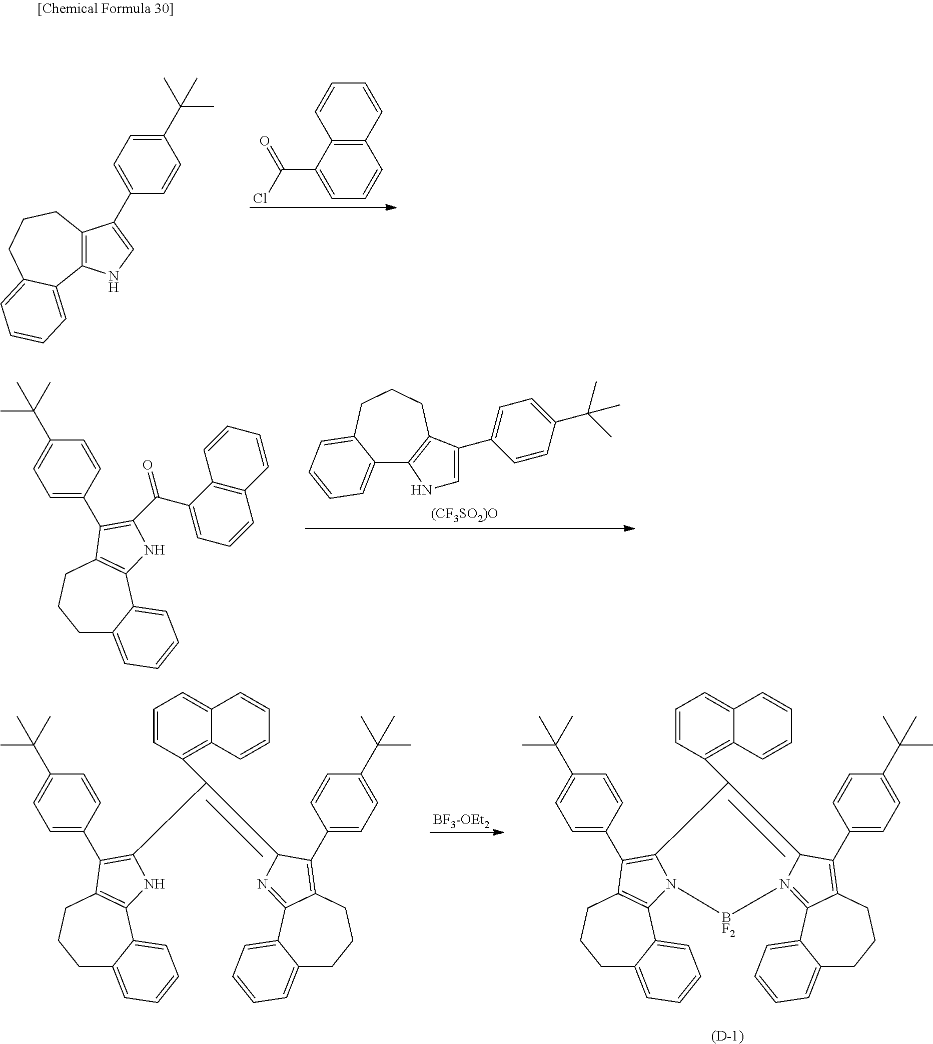

### Synthesis Example 2

Method of Synthesizing Compound D-2

To a mixed solution of 0.36 g of 3-(4-tert-butylphenyl)-1,4,5,6-tetrahydrobenzo[6,7]cyclohepta[1,2-b]pyrrole, 0.09 g of 2,4,6-trimethylbenzaldehyde, and 30 ml of dichloromethane, 2 drops of trifluoroacetic acid was added, and the resulting mixture was stirred at room temperature for 2 hours under a nitrogen stream. After that, 50 ml of water was added, and the resulting mixture was subjected to extraction with 50 ml of dichloromethane. The organic layer was washed with 50 ml of water, then magnesium sulfate was added, and the resulting mixture was filtered. The solvent was removed from the filtrate with an evaporator to obtain 0.38 g of a pyrromethane compound.

Next, to 0.38 g of the obtained pyrromethane compound, 0.15 g of 2,3-dichloro-5,6-dicyano-p-benzoquinone (DDQ) and 20 ml of dichloromethane were added, and the resulting mixture was stirred at room temperature for 4 hours. After confirming disappearance of the pyrromethane compound with LC-MS, 0.75 ml of N,N-diisopropylethylamine and 0.60 ml of boron trifluoride diethyl ether complex were added, and the resulting mixture was stirred at room temperature for 8 hours. After that, 50 ml of water was added, and the resulting mixture was subjected to extraction with 50 ml of dichloromethane. The organic layer was washed with 50 ml of water, then magnesium sulfate was added, and the resulting mixture was filtered. The solvent was removed from the filtrate with an evaporator, and then the residue was purified with silica gel column chromatography (heptane/toluene=1/2). Furthermore, to the concentrated purified product, 50 ml of methanol was added, and the resulting mixture was heated and stirred at 60° C. for 10 minutes and then cooled. The precipitated solid was filtered out and vacuum-dried to obtain 0.26 g of a reddish purple powder. The obtained powder was analyzed with LC-MS, and it was confirmed that the reddish purple powder was a compound D-2 as a pyrromethene metal complex.

MS (m/z) 723 [M+H]⁺ Compound D-2:

The compound D-2 was subjected to sublimation purification using an oil diffusion pump under a pressure of 1×10⁻³ Pa at 270° C. and then used as a light-emitting element material.

The light emission characteristics in the solution of the compound D-2 are shown below.

Absorption spectrum (solvent: toluene): λmax 582 nm

Fluorescence spectrum (solvent: toluene): λmax 605 nm, half-value width 35 nm

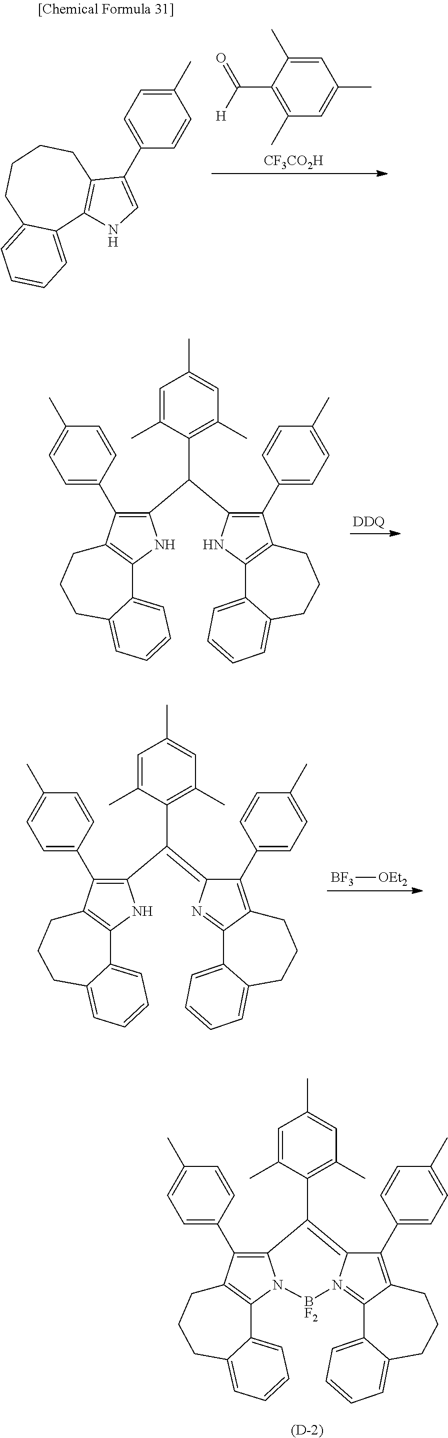

Pyrromethene metal complexes used in Examples and Comparative Examples described below are the compounds shown below. Table 1 shows the light emission characteristics of these pyrromethene metal complex compounds in a toluene solution.

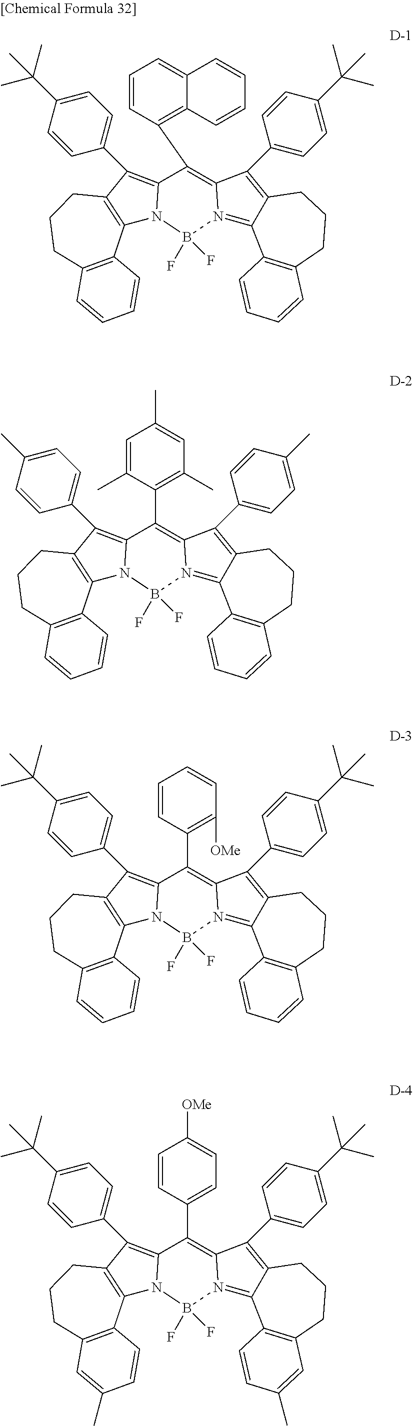

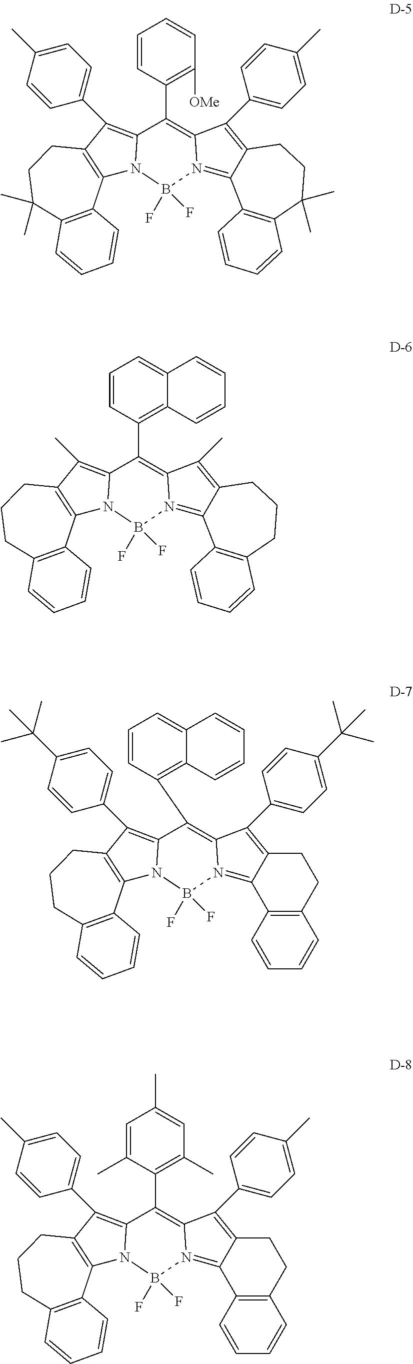

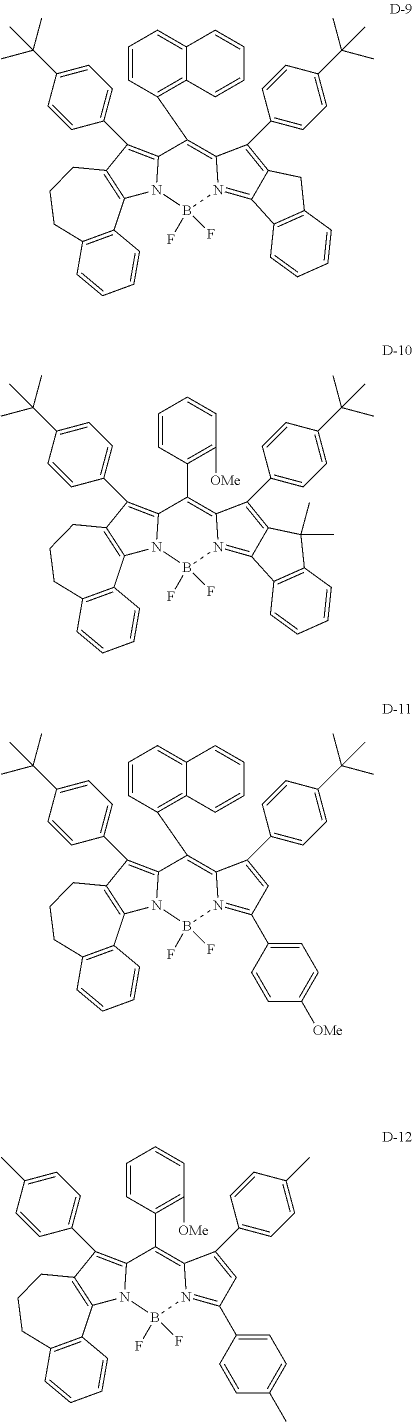

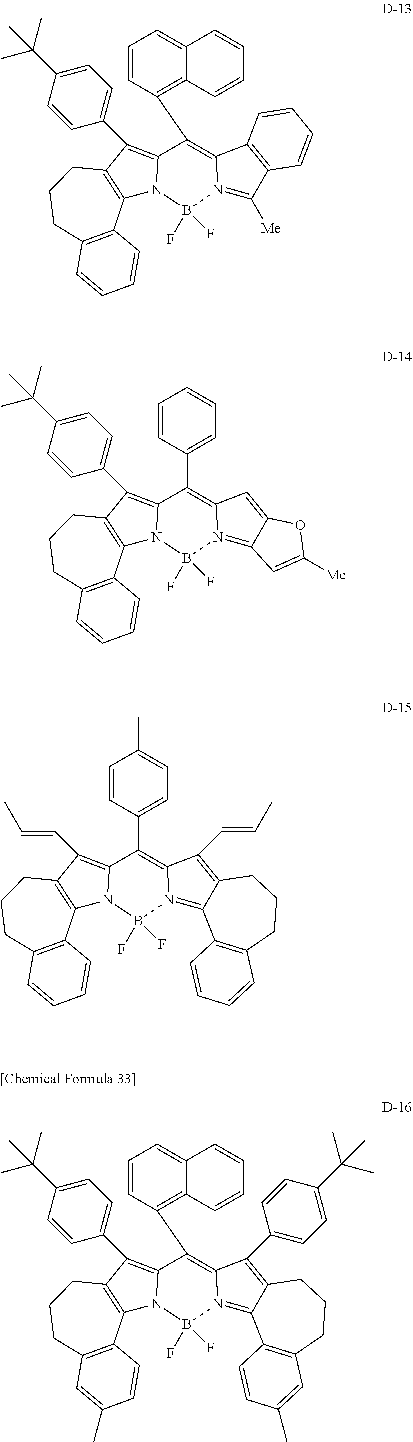

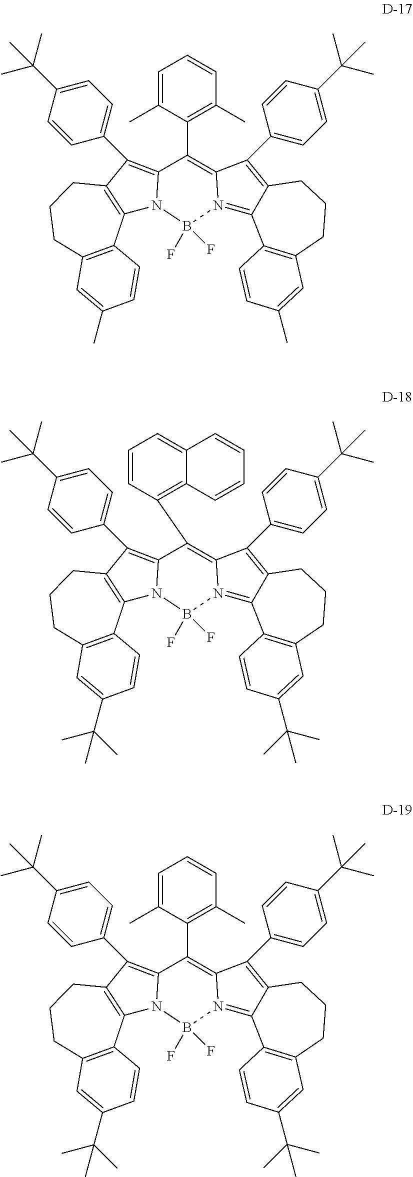

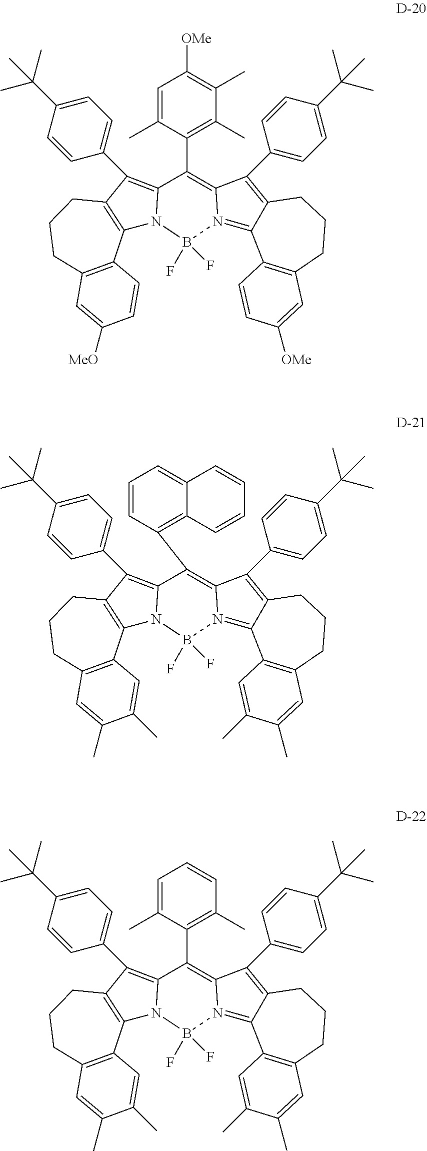

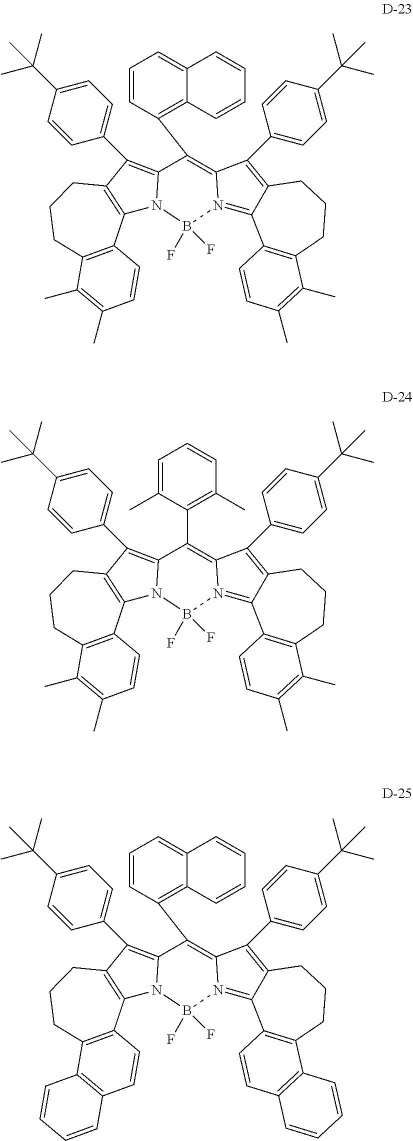

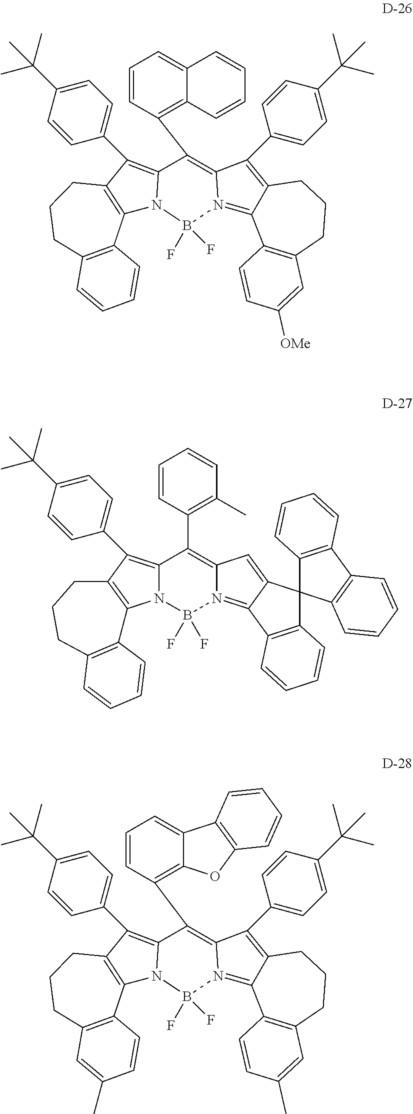

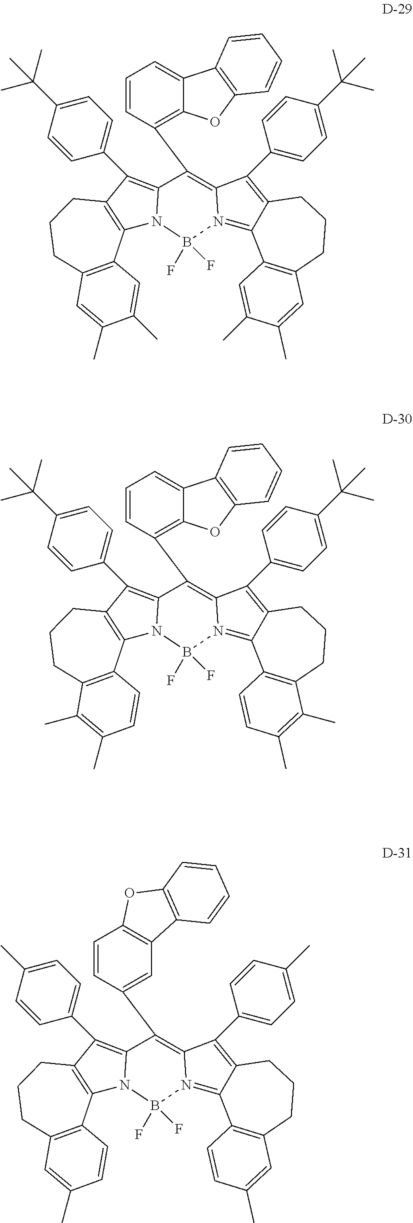

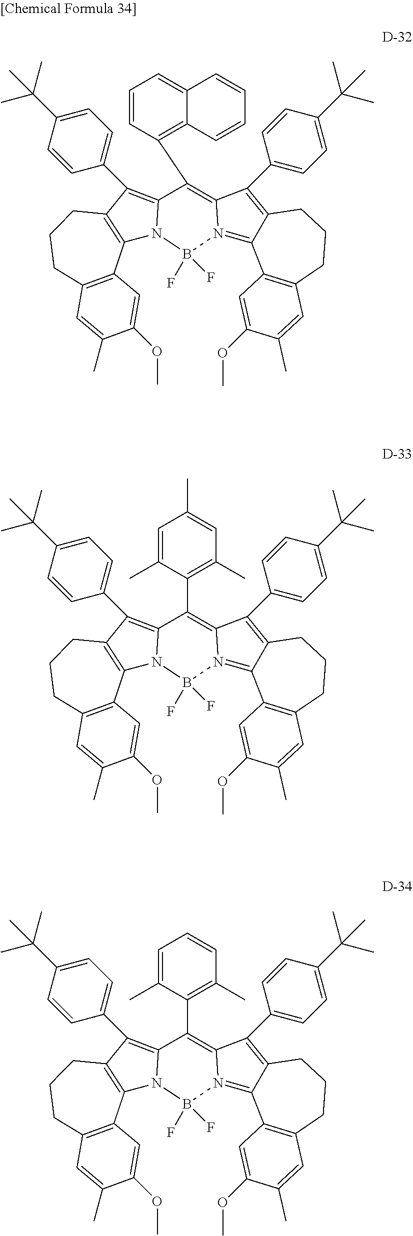

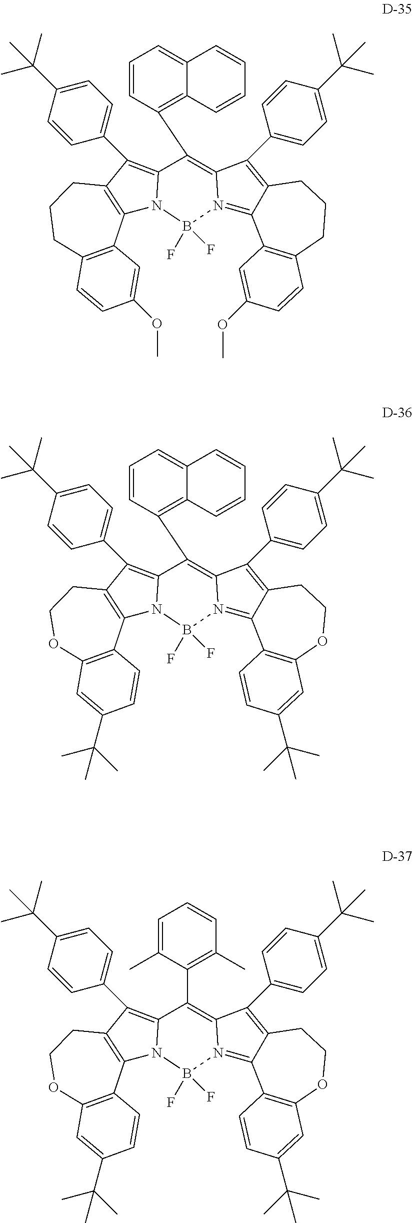

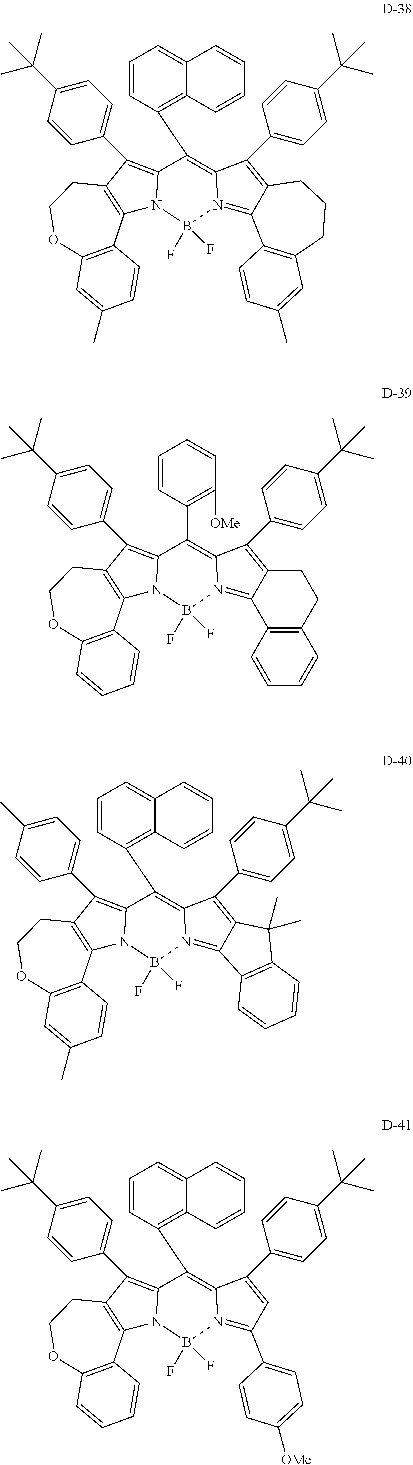

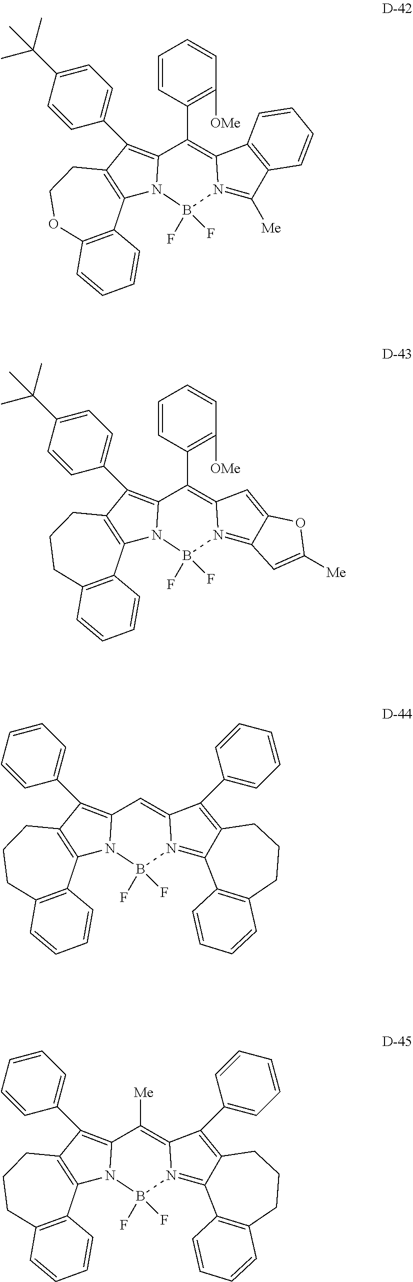

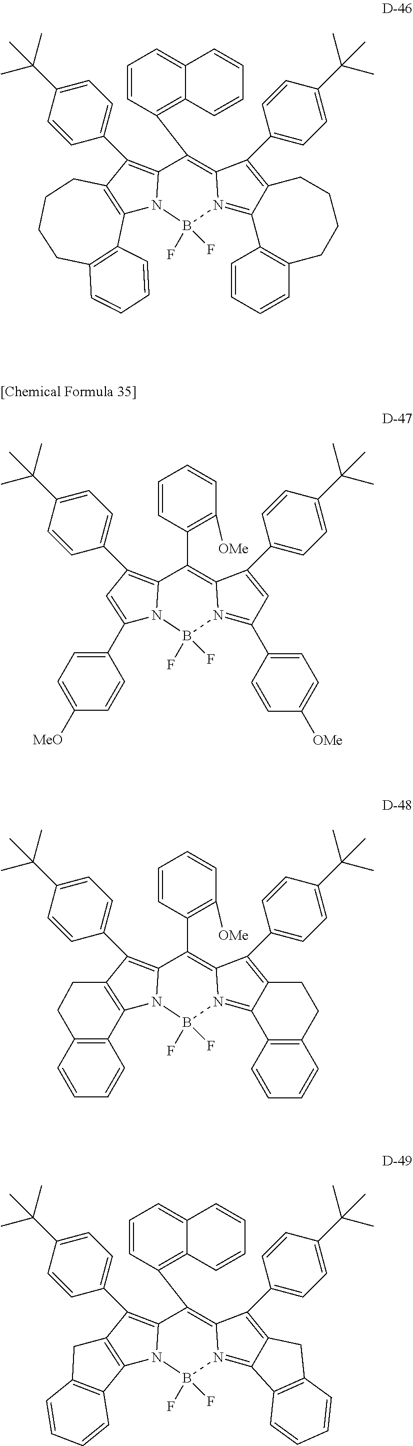

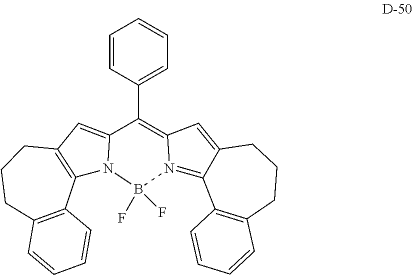

TABLE 1-1

| | Absorption | Emission spectrum | |
|---|---|---|---|
| | spectrum | | Half-value |
| Compound | λmax (nm) | λmax (nm) | width (nm) |
| D-1 | 584 | 607 | 35 |
| D-2 | 582 | 604 | 33 |
| D-3 | 584 | 607 | 35 |
| D-4 | 597 | 617 | 34 |
| D-5 | 585 | 609 | 35 |
| D-6 | 569 | 592 | 35 |
| D-7 | 608 | 628 | 32 |
| D-8 | 605 | 625 | 30 |
| D-9 | 605 | 623 | 27 |
| D-10 | 606 | 624 | 29 |
| D-11 | 589 | 617 | 36 |
| D-12 | 583 | 611 | 36 |
| D-13 | 610 | 635 | 32 |
| D-14 | 588 | 613 | 30 |
| D-15 | 594 | 617 | 36 |
| D-16 | 590 | 613 | 35 |
| D-17 | 588 | 612 | 35 |
| D-18 | 591 | 616 | 32 |
| D-19 | 590 | 615 | 33 |
| D-20 | 598 | 624 | 35 |
| D-21 | 590 | 616 | 32 |
| D-22 | 589 | 615 | 34 |
| D-23 | 591 | 617 | 33 |
| D-24 | 590 | 615 | 34 |
| D-25 | 600 | 624 | 35 |
| D-26 | 593 | 617 | 33 |
| D-27 | 610 | 629 | 25 |
| D-28 | 592 | 618 | 33 |
| D-29 | 595 | 620 | 34 |
| D-30 | 595 | 621 | 33 |
| D-31 | 593 | 619 | 34 |
| D-32 | 597 | 621 | 32 |
| D-33 | 595 | 620 | 35 |
| D-34 | 594 | 619 | 33 |
| D-35 | 594 | 619 | 35 |
| D-36 | 593 | 617 | 36 |
| D-37 | 591 | 618 | 36 |
| D-38 | 592 | 618 | 35 |
| D-39 | 609 | 630 | 34 |
| D-40 | 607 | 626 | 31 |
| D-41 | 592 | 620 | 37 |
| D-42 | 612 | 637 | 35 |
| D-43 | 593 | 618 | 33 |
| D-44 | 591 | 614 | 35 |
| D-45 | 587 | 611 | 35 |
| D-46 | 570 | 604 | 38 |
| D-47 | 590 | 630 | 42 |
| D-48 | 634 | 650 | 27 |
| D-49 | 636 | 648 | 25 |
| D-50 | 575 | 598 | 34 |

### Example 1

(Evaluation of Fluorescent Bottom Emission Type Light-Emitting Element)

A glass substrate on which an ITO transparent electroconductive film was deposited in a thickness of 165 nm (manufactured by GEOMATEC Co., Ltd., 11Ω/□, sputtered product) was cut into 38×46 mm, and etched. The resulting substrate was ultrasonically washed with “Semico Clean 56” (trade name, manufactured by Furuuchi Chemical Corporation) for 15 minutes, and then washed with ultrapure water. This substrate was treated with UV-ozone for 1 hour immediately before preparation of an element, and placed in a vacuum deposition apparatus, and the apparatus was evacuated until the degree of vacuum in the apparatus was 5×10⁻⁴ Pa or less. With a resistance heating method, first, HAT-CN6 was deposited as a hole injection layer in a thickness of 5 nm, and then HT-1 was deposited as a hole transporting layer in a thickness of 50 nm. Next, as an emissive layer, H-1 (a first compound) as a host material and a compound D-1 (second compound) as a dopant material were deposited in a thickness of 20 nm so that the doping concentration was 0.5% by weight. Furthermore, using ET-1 as an electron transporting layer and 2E-1 as a donor material, the ET-1 and the 2E-1 were stacked to a thickness of 35 nm so that the deposition rate ratio between the ET-1 and the 2E-1 was 1:1. Next, 2E-1 was deposited in a thickness of 0.5 nm as an electron injection layer, then magnesium and silver were co-deposited in a thickness of 1,000 nm to form a cathode, and thus a 5×5 mm square bottom emission type light-emitting element was prepared.

In light emission at 1,000 cd/m², this light-emitting element had light emission characteristics of a light emission peak wavelength of 611 nm, a half-value width of 38 nm, and an external quantum efficiency of 5.8%. Regarding the durability, the light-emitting element was continuously energized with a current at which the initial luminance was 1,000 cd/m², and the durability was evaluated with the time at which the luminance was 90% of the initial luminance (hereinafter, referred to as LT90). As a result, the LT90 of the light-emitting element was 245 hours. HAT-CN6, HT-1, H-1, ET-1, and 2E-1 are compounds shown below.

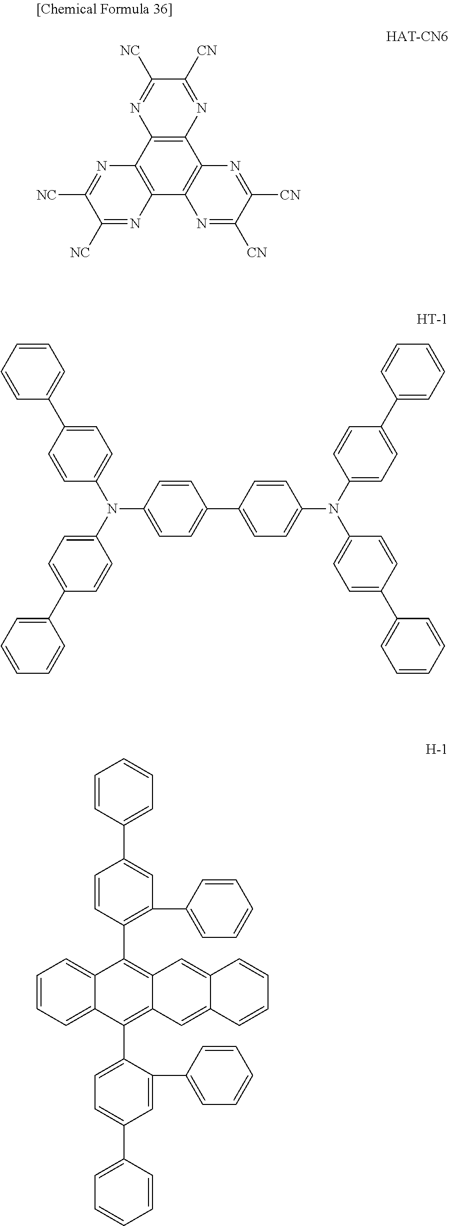

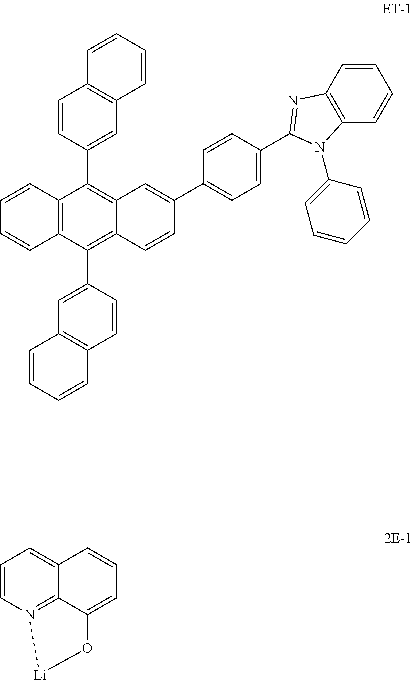

### Examples 2 to 46 and Comparative Examples 1 to 4

A light-emitting element was prepared and evaluated in the same manner as in Example 1 except that compounds described in Table 1 were used as a dopant material. Table 2 shows the results.

TABLE 2-1

| | | Light | | | |
|---|---|---|---|---|---|
| | | emission | Half- | External | |
| | | peak | value | quantum | |
| | | wavelength | width | efficiency | LT90 |
| | Compound | (nm) | (nm) | (%) | (Time) |
| Example 1 | D-1 | 611 | 38 | 5.8 | 245 |
| Example 2 | D-2 | 608 | 36 | 6.2 | 232 |
| Example 3 | D-3 | 611 | 38 | 5.9 | 214 |
| Example 4 | D-4 | 621 | 37 | 5.3 | 225 |
| Example 5 | D-5 | 613 | 38 | 5.8 | 301 |
| Example 6 | D-6 | 596 | 38 | 3.9 | 127 |
| Example 7 | D-7 | 632 | 35 | 6.2 | 247 |
| Example 8 | D-8 | 629 | 33 | 6.4 | 227 |
| Example 9 | D-9 | 627 | 30 | 6.1 | 242 |
| Example 10 | D-10 | 628 | 32 | 6.0 | 270 |
| Example 11 | D-11 | 622 | 39 | 5.6 | 237 |
| Example 12 | D-12 | 615 | 39 | 5.4 | 212 |
| Example 13 | D-13 | 639 | 36 | 5.0 | 154 |
| Example 14 | D-14 | 617 | 33 | 5.0 | 132 |
| Example 15 | D-15 | 621 | 39 | 3.7 | 52 |
| Example 16 | D-16 | 617 | 38 | 6.2 | 250 |
| Example 17 | D-17 | 616 | 38 | 6.7 | 225 |
| Example 18 | D-18 | 620 | 35 | 6.2 | 275 |
| Example 19 | D-19 | 619 | 36 | 6.7 | 243 |
| Example 20 | D-20 | 628 | 38 | 6.6 | 213 |
| Example 21 | D-21 | 620 | 35 | 6.2 | 255 |
| Example 22 | D-22 | 619 | 37 | 6.7 | 236 |
| Example 23 | D-23 | 621 | 36 | 6.2 | 258 |
| Example 24 | D-24 | 619 | 37 | 6.7 | 226 |
| Example 25 | D-25 | 628 | 38 | 6.2 | 248 |
| Example 26 | D-26 | 621 | 36 | 6.0 | 231 |
| Example 27 | D-27 | 633 | 28 | 6.0 | 214 |
| Example 28 | D-28 | 621 | 36 | 6.2 | 248 |
| Example 29 | D-29 | 623 | 37 | 6.3 | 257 |
| Example 30 | D-30 | 624 | 36 | 6.4 | 253 |
| Example 31 | D-31 | 622 | 37 | 5.1 | 211 |
| Example 32 | D-32 | 624 | 35 | 6.0 | 238 |
| Example 33 | D-33 | 623 | 38 | 6.7 | 224 |
| Example 34 | D-34 | 623 | 36 | 6.7 | 225 |
| Example 35 | D-35 | 623 | 38 | 5.4 | 215 |
| Example 36 | D-36 | 621 | 39 | 6.0 | 214 |
| Example 37 | D-37 | 622 | 39 | 6.6 | 201 |
| Example 38 | D-38 | 622 | 38 | 6.0 | 218 |
| Example 39 | D-39 | 634 | 37 | 5.5 | 207 |
| Example 40 | D-40 | 630 | 34 | 5.5 | 205 |
| Example 41 | D-41 | 624 | 39 | 5.8 | 200 |
| Example 42 | D-42 | 641 | 38 | 4.7 | 155 |
| Example 43 | D-43 | 622 | 36 | 4.9 | 136 |
| Example 44 | D-44 | 618 | 38 | 5.8 | 143 |
| Example 45 | D-45 | 615 | 38 | 6.0 | 140 |
| Example 46 | D-46 | 608 | 42 | 5.8 | 110 |
| Comparative | D-47 | 635 | 46 | 6.0 | 250 |
| Example 1 | | | | | |
| Comparative | D-48 | 654 | 30 | 6.2 | 204 |
| Example 2 | | | | | |
| Comparative | D-49 | 652 | 28 | 6.2 | 201 |
| Example 3 | | | | | |
| Comparative | D-50 | 602 | 37 | 2.8 | 50 |
| Example 4 | | | | | |

As can be seen with reference to Table 2, in Examples 1 to 46, light emission was obtained in which the half-value width was narrower than that of the non-bridging type light-emitting element in Comparative Example 1. In Comparative Examples 2 to 3, although the half-value width was narrow, the peak wavelength was 650 nm or more, so that obtained light was deep red and it was difficult to achieve chromaticity for use in a display device or an illumination device. In Comparative Example 4, although the half-value width was narrow, the external quantum efficiency and the durability were low.

### Example 47

(Evaluation of TADF Bottom Emission Type Light-Emitting Element)

A glass substrate on which an ITO transparent electroconductive film was deposited in a thickness of 165 nm (manufactured by GEOMATEC Co., Ltd., 11Ω/□, sputtered product) was cut into 38×46 mm, and etched. The resulting substrate was ultrasonically washed with “Semico Clean 56” (trade name, manufactured by Furuuchi Chemical Corporation) for 15 minutes, and then washed with ultrapure water. This substrate was treated with UV-ozone for 1 hour immediately before preparation of an element, and placed in a vacuum deposition apparatus, and the apparatus was evacuated until the degree of vacuum in the apparatus was 5×10⁻⁴ Pa or less. With a resistance heating method, first, HAT-CN6 was deposited as a hole injection layer in a thickness of 10 nm, and then HT-1 was deposited as a hole transporting layer in a thickness of 180 nm. Next, as an emissive layer, a host material H-2 (third compound), a compound D-1 (second compound), and a compound H-3 as a TADF material (first compound) were deposited at a weight ratio of 80:0.5:19.5 in a thickness of 40 nm. Furthermore, as an electron transporting layer, using a compound ET-1 as an electron transporting material and a compound 2E-1 as a donor material, the compound ET-1 and the compound 2E-1 were stacked to a thickness of 35 nm so that the deposition rate ratio between the compound ET-1 and the compound 2E-1 was 1:1. Next, 2E-1 was deposited in a thickness of 0.5 nm as an electron injection layer, then magnesium and silver were co-deposited in a thickness of 1,000 nm to form a cathode, and thus a 5×5 mm square bottom emission type light-emitting element was prepared.

In light emission at 1,000 cd/m², this light-emitting element had light emission characteristics of a light emission peak wavelength of 612 nm, a half-value width of 38 nm, an external quantum efficiency of 13.2%, and an LT90 of 172 hours. H-2 and H-3 are compounds shown below.

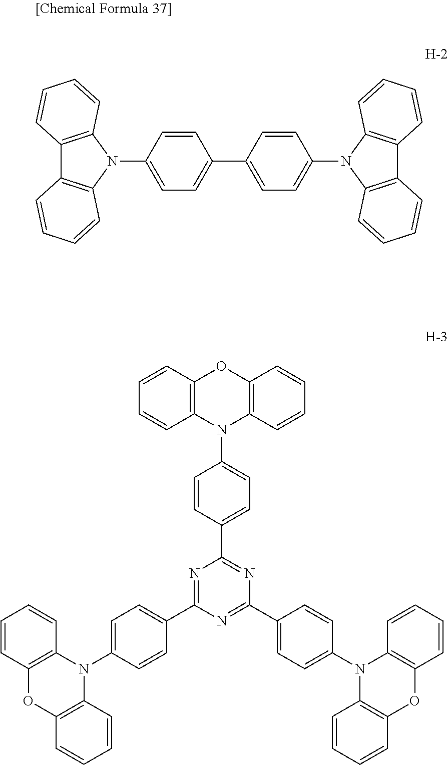 [SMILES: c1ccc2c(c1)c1ccccc1n2-c1ccc(-c2ccc(-n3c4ccccc4c4ccccc43)cc2)cc1 ; c1ccc2c(c1)Oc1ccccc1N2c1ccc(-c2nc(-c3ccc(N4c5ccccc5Oc5ccccc54)cc3)nc(-c3ccc(N4c5ccccc5Oc5ccccc54)cc3)n2)cc1]

The singlet excitation energy level S₁ and the triplet excitation energy level T₁ of each of the compounds H-2 and H-3 are as follows.

S₁ (H-2): 3.4 eV

T₁ (H-2): 2.6 eV

S₁ (H-3): 2.3 eV

T₁ (H-3): 2.2 eV

### Examples 48 to 72 and Comparative Examples 5 to 6

A light-emitting element was prepared and evaluated in the same manner as in Example 47 except that compounds described in Table 3 were used as a dopant material. Table 3 shows the results.

TABLE 3

| | | Light | | | |
|---|---|---|---|---|---|
| | | emission | Half- | External | |
| | | peak | value | quantum | |
| | | wavelength | width | efficiency | LT90 |
| | Compound | (nm) | (nm) | (%) | (Time) |
| Example 47 | D-1 | 612 | 38 | 13.2 | 172 |
| Example 48 | D-7 | 632 | 35 | 15.1 | 174 |
| Example 49 | D-10 | 629 | 32 | 14.6 | 189 |
| Example 50 | D-11 | 622 | 39 | 12.8 | 166 |
| Example 51 | D-16 | 617 | 38 | 15.5 | 177 |
| Example 52 | D-17 | 616 | 38 | 18.1 | 158 |
| Example 53 | D-18 | 620 | 35 | 15.8 | 195 |
| Example 54 | D-19 | 619 | 36 | 18.0 | 170 |
| Example 55 | D-20 | 627 | 38 | 17.4 | 150 |
| Example 56 | D-21 | 620 | 35 | 15.6 | 179 |
| Example 57 | D-22 | 619 | 37 | 18.1 | 165 |
| Example 58 | D-23 | 621 | 36 | 15.5 | 180 |
| Example 59 | D-24 | 619 | 37 | 18.2 | 158 |
| Example 60 | D-25 | 628 | 38 | 15.4 | 171 |
| Example 61 | D-26 | 621 | 36 | 14.7 | 162 |
| Example 62 | D-27 | 633 | 28 | 14.8 | 150 |
| Example 63 | D-28 | 621 | 36 | 15.4 | 179 |
| Example 64 | D-29 | 623 | 37 | 15.9 | 193 |
| Example 65 | D-30 | 624 | 36 | 16.4 | 183 |
| Example 66 | D-31 | 622 | 37 | 11.6 | 142 |
| Example 67 | D-32 | 624 | 35 | 14.4 | 173 |
| Example 68 | D-33 | 623 | 38 | 18.2 | 159 |
| Example 69 | D-34 | 623 | 36 | 18.1 | 161 |
| Example 70 | D-35 | 623 | 38 | 12.4 | 150 |
| Example 71 | D-28 | 621 | 39 | 14.6 | 148 |
| Example 72 | D-29 | 622 | 39 | 17.5 | 141 |
| Comparative | D-39 | 637 | 47 | 11.8 | 175 |
| Example 5 | | | | | |
| Comparative | D-42 | 603 | 37 | 6.4 | 35 |
| Example 6 | | | | | |

### Example 6

As can be seen with reference to Table 3, in Examples 47 to 72 and Comparative Examples 5 to 6, a TADF material was used in the emissive layer, and therefore the external quantum efficiency was significantly improved as compared with 1 to 46 and Comparative Examples 1 to 4. Among Examples and Comparative Examples, in Examples 47 to 72, the half-value width was narrow, and highly efficient light emission was obtained. Meanwhile, in Comparative Example 5, although the external quantum efficiency was high, the half-value width was wide. In Comparative Example 6, although the half-value width was narrow, the external quantum efficiency was low.

### Example 73

(Evaluation of TADF Top Emission Type Light-Emitting Element)

A glass substrate on which a metallic aluminum reflection film having a thickness of 100 nm and an ITO transparent electroconductive film having a thickness of 50 nm were deposited in this order (manufactured by GEOMATEC Co., Ltd., 11Ω/□, sputtered product) was cut into 38×46 mm, and etched. The resulting substrate was ultrasonically washed with “Semico Clean 56” (trade name, manufactured by Furuuchi Chemical Corporation) for 15 minutes, and then washed with ultrapure water. This substrate was treated with UV-ozone for 1 hour immediately before preparation of an element, and placed in a vacuum deposition apparatus, and the apparatus was evacuated until the degree of vacuum in the apparatus was 5×10⁻⁴ Pa or less. With a resistance heating method, on the ITO transparent electroconductive film, first, HAT-CN6 was deposited as a hole injection layer in a thickness of 10 nm, and then HT-1 was deposited as a hole transporting layer in a thickness of 125 nm. Next, as an emissive layer, a host material H-2 (third compound), a compound D-1 (second compound), and a compound H-3 as a TADF material (first compound) were deposited at a weight ratio of 80:0.5:19.5 in a thickness of 20 nm. Furthermore, as an electron transporting layer, using a compound ET-1 as an electron transporting material and a compound 2E-1 as a donor material, the compound ET-1 and the compound 2E-1 were stacked to a thickness of 30 nm so that the deposition rate ratio between the compound ET-1 and the compound 2E-1 was 1:1. Next, 2E-1 was deposited in a thickness of 1 nm as an electron injection layer, then magnesium and silver were co-deposited in a thickness of 20 nm to form a cathode, and thus a 5×5 mm square top emission type light-emitting element was prepared.

In light emission at 1,000 cd/m², this light-emitting element had light emission characteristics of a light emission peak wavelength of 615 nm, a half-value width of 33 nm, a CIE chromaticity (x, y=0.66, 0.34), a current efficiency of 42 cd/A, and an LT90 of 172 hours.

### Examples 74 to 81 and Comparative Example 7

A light-emitting element was prepared and evaluated in the same manner as in Example 73 except that compounds described in Table 4 were used as a dopant material. Table 4 shows the results.

TABLE 4

| | | Light | | | | | |
|---|---|---|---|---|---|---|---|
| | | emission | Half-value | | | Current | |
| | | peak wavelength | width | CIE chromaticity | | efficiency | LT90 |
| | Compound | (nm) | (nm) | x | y | (cd/A) | (Time) |
| Example 73 | D-16 | 615 | 33 | 0.66 | 0.34 | 44 | 172 |
| Example 74 | D-18 | 618 | 31 | 0.68 | 0.32 | 43 | 190 |
| Example 75 | D-21 | 618 | 31 | 0.68 | 0.32 | 42 | 175 |
| Example 76 | D-23 | 619 | 32 | 0.68 | 0.32 | 42 | 177 |
| Example 77 | D-26 | 619 | 32 | 0.69 | 0.31 | 40 | 158 |
| Example 78 | D-28 | 619 | 32 | 0.69 | 0.31 | 43 | 175 |
| Example 79 | D-29 | 621 | 33 | 0.69 | 0.32 | 45 | 187 |
| Example 80 | D-30 | 622 | 32 | 0.69 | 0.32 | 46 | 176 |
| Example 81 | D-32 | 622 | 32 | 0.69 | 0.32 | 37 | 168 |
| Comparative | D-39 | 635 | 36 | 0.70 | 0.30 | 16 | 171 |
| Example 7 | | | | | | | |

As can be seen with reference to Table 4, in Examples 73 to 81 and Comparative Example 7, an emission spectrum having a narrow half-value width was obtained. Meanwhile, in Examples 73 to 81, higher current efficiency was obtained as compared with Comparative Example 7. In the top emission type light-emitting element, light in the wavelength region in which resonance occurs is mutually intensified due to the cavity effect, but light having a wavelength outside this region is mutually weakened. Therefore, the current efficiency is high in a light-emitting element in which an emissive material having an emission spectrum having a narrow half-value width is used, and the effect was confirmed.

### Example 82

(Measurement of Light Emission Characteristics of Doped Thin Film)

A quartz glass plate (10×10 mm) was ultrasonically washed with “Semico Clean 56” (trade name, manufactured by Furuuchi Chemical Corporation) for 15 minutes, then washed with ultrapure water, and dried. This glass plate was treated with UV-ozone for 1 hour immediately before preparation of an element, and placed in a vacuum deposition apparatus, and the apparatus was evacuated until the degree of vacuum in the apparatus was 5×10⁻⁴ Pa or less. With a resistance heating method, mCBP as a host material and a compound D-1 as a dopant material were deposited in a thickness of 500 nm so that the doping concentration was 1% by weight to obtain a 1% by weight doped thin film. A 2% by weight doped thin film and a 4% by weight doped thin film were obtained with the same method.

Light emission characteristics of the 1% by weight doped thin film are shown.

Light Emission Peak Wavelength: λMax 611 nm, Half-Value Width 38 nm

For each of the 1% by weight, 2% by weight, and 4% by weight doped thin films, the fluorescence quantum yield at an excitation light wavelength of 540 nm was determined using a fluorescence quantum yield measurement apparatus C 11347-01 (manufactured by Hamamatsu Photonics K.K.). The fluorescence quantum yield at a doping concentration of 1% was regarded as 1, and at that time, the ratio of the fluorescence quantum yield at each doping concentration was determined as the QY ratio using the following formula.

QY ratio=(fluorescence quantum yield of thin film having doping concentration of x % by weight)/(fluorescence quantum yield of thin film having doping concentration of 1% by weight)

$$[ x = 1 , 2 , or 4 ]$$

The results are shown below.

Doping concentration 1% by weight; fluorescence quantum yield 70%, QY ratio=1

Doping concentration 2% by weight; fluorescence quantum yield 59%, QY ratio=0.84

Doping concentration 4% by weight; fluorescence quantum yield 49%, QY ratio=0.70

### Examples 83 to 99

The fluorescence quantum yield and the QY ratio of the doped thin film were determined in the same manner as in Example 82 except that compounds described in Table 5 were used as a dopant material. Table 5 shows the results.

TABLE 5

| | | Emission spectrum | | | | | | | |
|---|---|---|---|---|---|---|---|---|---|
| | | [1% doped thin film] | | Fluorescence quantum yield | | | | | |
| | | | Half-value | [thin film] (%) | | | QY ratio | | |
| Example | Compound | λmax (nm) | width (nm) | 1% doped | 2% doped | 4% doped | 1% doped | 2% doped | 4% doped |
| Example 82 | D-1 | 611 | 38 | 70 | 59 | 49 | 1 | 0.84 | 0.70 |
| Example 83 | D-2 | 608 | 36 | 75 | 72 | 66 | 1 | 0.96 | 0.88 |
| Example 84 | D-3 | 611 | 38 | 70 | 58 | 47 | 1 | 0.83 | 0.67 |
| Example 85 | D-16 | 617 | 38 | 72 | 63 | 51 | 1 | 0.88 | 0.71 |
| Example 86 | D-17 | 616 | 38 | 80 | 78 | 72 | 1 | 0.98 | 0.90 |
| Example 87 | D-18 | 620 | 35 | 78 | 66 | 55 | 1 | 0.85 | 0.71 |
| Example 88 | D-19 | 619 | 36 | 82 | 78 | 72 | 1 | 0.95 | 0.88 |
| Example 89 | D-20 | 628 | 38 | 80 | 77 | 70 | 1 | 0.96 | 0.88 |
| Example 90 | D-21 | 620 | 35 | 78 | 65 | 54 | 1 | 0.83 | 0.69 |
| Example 91 | D-22 | 619 | 37 | 82 | 77 | 70 | 1 | 0.94 | 0.85 |
| Example 92 | D-23 | 621 | 36 | 77 | 64 | 54 | 1 | 0.83 | 0.70 |
| Example 93 | D-24 | 619 | 37 | 81 | 77 | 70 | 1 | 0.95 | 0.86 |
| Example 94 | D-28 | 621 | 36 | 72 | 65 | 54 | 1 | 0.90 | 0.75 |
| Example 95 | D-29 | 623 | 37 | 74 | 67 | 57 | 1 | 0.91 | 0.77 |
| Example 96 | D-30 | 624 | 36 | 76 | 68 | 57 | 1 | 0.89 | 0.75 |
| Example 97 | D-32 | 624 | 35 | 71 | 64 | 52 | 1 | 0.90 | 0.73 |
| Example 98 | D-33 | 623 | 38 | 82 | 77 | 70 | 1 | 0.94 | 0.85 |
| Example 99 | D-34 | 623 | 36 | 80 | 75 | 68 | 1 | 0.94 | 0.85 |

From the comparison of the QY ratios in Table 5, it has been found that in each of Example 83, Example 86, Example 88, Example 89, Example 91, Example 93, Example 98, and Example 99 in which a pyrromethene metal complex was used that had a phenyl group, at the bridge-head position, having substituents at both the second position and the sixth position with respect to the bonding moiety with the pyrromethene skeleton, the decrease in the fluorescence quantum yield due to the increase in the doping concentration is small, that is, the concentration quenching is small, as compared with the case in which another pyrromethene metal complex was used.

As described above, it has been shown that by using the pyrromethene metal complex of the present invention, a light-emitting element can be prepared that has high external quantum efficiency and an emission spectrum having a narrow half-value width. Furthermore, it has been found that the current efficiency is significantly improved in the top emission type light-emitting element. Although emission of red light having a light emission peak wavelength of 640 nm or less has been conventionally difficult to achieve, it has been found that the emission can be obtained, and thus the design range of the wavelength can be widened. As a result, it has been shown that the color control is facilitated and the color purity and the luminous efficiency can be enhanced in production of display devices such as a display and illumination devices.

### Example 100

(Evaluation of TADF Bottom Emission Type Light-Emitting Element in which Two Host Materials are Used)

A glass substrate on which an ITO transparent electroconductive film was deposited in a thickness of 165 nm (manufactured by GEOMATEC Co., Ltd., 11Ω/□, sputtered product) was cut into 38×46 mm, and etched. The resulting substrate was ultrasonically washed with “Semico Clean 56” (trade name, manufactured by Furuuchi Chemical Corporation) for 15 minutes, and then washed with ultrapure water. This substrate was treated with UV-ozone for 1 hour immediately before preparation of an element, and placed in a vacuum deposition apparatus, and the apparatus was evacuated until the degree of vacuum in the apparatus was 5×10⁻⁴ Pa or less. With a resistance heating method, first, HAT-CN6 was deposited as a hole injection layer in a thickness of 10 nm, and then HT-1 was deposited as a hole transporting layer in a thickness of 180 nm. Next, as an emissive layer, a first host material H-2 (hole transporting third compound), a second host material H-4 (electron transporting third compound), a compound D-1 (second compound), and a compound H-3 as a TADF material (first compound) were deposited at a weight ratio of 40:40:0.5:19.5 in a thickness of 40 nm. Furthermore, as an electron transporting layer, using a compound ET-1 as an electron transporting material and a compound 2E-1 as a donor material, the compound ET-1 and the compound 2E-1 were stacked to a thickness of 35 nm so that the deposition rate ratio between the compound ET-1 and the compound 2E-1 was 1:1. Next, 2E-1 was deposited in a thickness of 0.5 nm as an electron injection layer, then magnesium and silver were co-deposited in a thickness of 1,000 nm to form a cathode, and thus a 5×5 mm square bottom emission type light-emitting element was prepared.

In light emission at 1,000 cd/m², this light-emitting element had light emission characteristics of a light emission peak wavelength of 612 nm, a half-value width of 38 nm, an external quantum efficiency of 13.0%, and an LT90 of 255 hours. It was confirmed that the light emission peak wavelength, the half-value width, and the external quantum efficiency were equivalent to those in Example 47 in which one host material was used, and as compared with Example 47, the LT90 was increased by about 1.5 times, and the durability was improved. H-4 is a compound shown below.

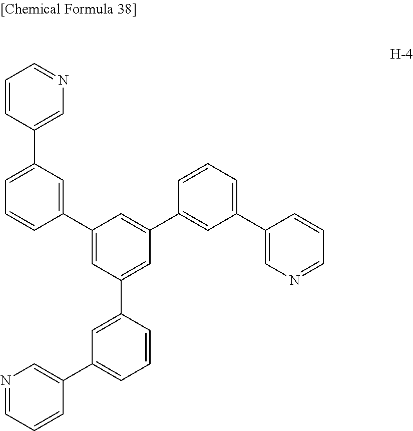 [SMILES: c1cncc(-c2cccc(-c3cc(-c4cccc(-c5cccnc5)c4)cc(-c4cccc(-c5cccnc5)c4)c3)c2)c1]

The singlet excitation energy level S₁ and the triplet excitation energy level T₁ of each of H-2 and H-4 are as follows.

S₁ (H-2): 3.4 eV

T₁ (H-2): 2.6 eV

S₁ (H-4): 3.9 eV

T₁ (H-4): 2.8 eV

### Example 101

(Evaluation of Tandem Type Fluorescent Light-Emitting Element)

A glass substrate on which an ITO transparent electroconductive film was deposited in a thickness of 165 nm (manufactured by GEOMATEC Co., Ltd., 11Ω/□, sputtered product) was cut into 38×46 mm, and etched. The resulting substrate was ultrasonically washed with “Semico Clean 56” (trade name, manufactured by Furuuchi Chemical Corporation) for 15 minutes, and then washed with ultrapure water. This substrate was treated with UV-ozone for 1 hour immediately before preparation of an element, and placed in a vacuum deposition apparatus, and the apparatus was evacuated until the degree of vacuum in the apparatus was 5×10⁻⁴ Pa or less. With a resistance heating method, first, HAT-CN6 was deposited as a hole injection layer in a thickness of 5 nm, and then HT-1 was deposited as a hole transporting layer in a thickness of 50 nm. Next, as an emissive layer, H-1 (a first compound) as a host material and a compound D-1 (second compound) as a dopant material were deposited in a thickness of 20 nm so that the doping concentration was 0.5% by weight. Furthermore, as an electron transporting layer, using a compound ET-1 as an electron transporting material and a compound 2E-1 as a donor material, the compound ET-1 and the compound 2E-1 were stacked to a thickness of 35 nm so that the deposition rate ratio between the compound ET-1 and the compound 2E-1 was 1:1. Next, as an n-type charge generation layer, using a compound ET-2 as an n-type host and metallic lithium as an n-type dopant, the compound ET-2 and the metallic lithium were stacked to a thickness of 10 nm so that the deposition rate ratio between the compound ET-2 and the metallic lithium was 99:1. Furthermore, HAT-CN6 was stacked to a thickness of 10 nm as a p-type charge emissive layer. On the HAT-CN6 as a p-type charge emissive layer, in the same manner as described above, HT-1 was deposited as a hole transporting layer in a thickness of 50 nm, a thin film in which a host material H-1 was doped with 0.5% by weight of a compound D-1 was deposited as an emissive layer in a thickness of 20 nm, and a thin film including ET-1 and 2E-1 at a ratio of 1:1 was deposited as an electron transporting layer in a thickness of 35 nm in this order. Next, 2E-1 was deposited in a thickness of 0.5 nm as an electron injection layer, then magnesium and silver were co-deposited in a thickness of 1,000 nm to form a cathode, and thus a 5×5 mm square tandem type fluorescent light-emitting element was prepared.

In light emission at 1,000 cd/m², this light-emitting element had light emission characteristics of a light emission peak wavelength of 611 nm, a half-value width of 38 nm, an external quantum efficiency of 10.9%, and an LT90 of 511 hours. It was confirmed that as compared with Example 1 in which only one emissive layer was provided, both the external quantum efficiency and the LT90 were increased by about 2 times, and the luminous efficiency and the durability were improved. ET-2 is a compound shown below.

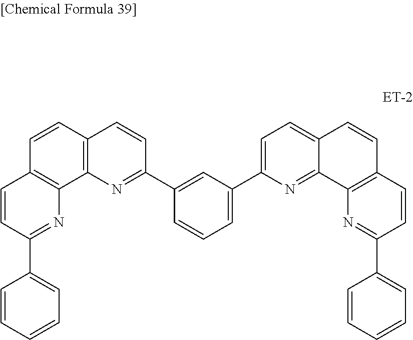 [SMILES: c1ccc(-c2ccc3ccc4ccc(-c5cccc(-c6ccc7ccc8ccc(-c9ccccc9)nc8c7n6)c5)nc4c3n2)cc1]

1. A pyrromethene metal complex represented by a general formula (1) or (2): 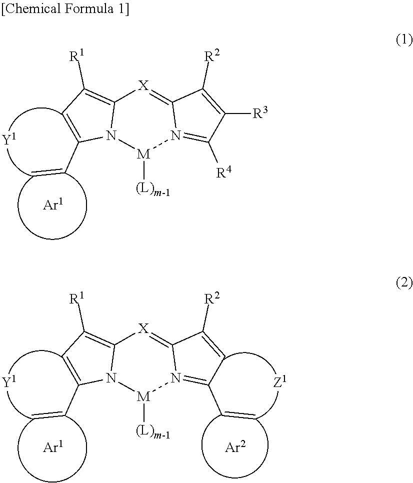
wherein X represents C—R⁵ or N,
R¹ to R⁵ are the same or different from each other, and are each selected from a hydrogen atom, an alkyl group, a cycloalkyl group, a heterocyclic group, an alkenyl group, a cycloalkenyl group, an alkynyl group, an aryl group, a heteroaryl group, a hydroxyl group, a thiol group, an alkoxy group, an alkylthio group, an aryl ether group, an aryl thioether group, a halogen, a cyano group, an aldehyde group, an acyl group, a carboxyl group, an ester group, an amide group, a sulfonyl group, a sulfonic acid ester group, a sulfonamide group, an amino group, a nitro group, a silyl group, a siloxanyl group, a boryl group, a phosphine oxide group, and a ring structure with an adjacent group, and the groups that are substituted, a ring structure formed by R³ and R⁴ is a monocyclic ring, R¹ is not a hydrogen atom and not a halogen in a case that Y¹ is a trimethylene group,
Ar¹ and Ar² are the same or different from each other, and are each selected from an aromatic hydrocarbon ring bonded to a substituent or no substituent and an aromatic heterocyclic ring bonded to a substituent or no substituent,
Y¹ is a bridging structure in which three or more atoms are bonded in series and form a double bond or no double bond with an adjacent atom, and the three or more atoms are selected from a carbon atom bonded to a substituent or no substituent, a silicon atom bonded to a substituent or no substituent, a nitrogen atom bonded to a substituent or no substituent, a phosphorus atom bonded to a substituent or no substituent, an oxygen atom, and a sulfur atom,
Z¹ is a bridging structure in which one or more atoms are bonded and form a double bond or no double bond with an adjacent atom, and the one or more atoms are selected from a carbon atom bonded to a substituent or no substituent, a silicon atom bonded to a substituent or no substituent, a nitrogen atom bonded to a substituent or no substituent, a phosphorus atom bonded to a substituent or no substituent, an oxygen atom, and a sulfur atom,
M is boron, and m is 3,
L is the same or different from each other, and are each selected from an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, and a cyano group, and the groups that are substituted.

2. (canceled)

3. The pyrromethene metal complex according to claim 1, wherein Y¹ is a bridging structure in which three atoms are bonded in series.

4. The pyrromethene metal complex according to claim 3, wherein Y¹ is represented by a general formula (5A) or (5B): 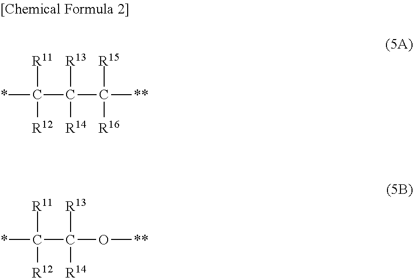
wherein * represents a linking moiety with a pyrrole ring, ** represents a linking moiety with Ar¹, R¹¹ to R¹⁶ are the same or different from each other, and are each selected from the groups from which R¹ to R⁵ in the general formula (1) or (2) are selected and an oxo group.

5. The pyrromethene metal complex according to claim 1, wherein X represents C—R⁵.

6. The pyrromethene metal complex according to claim 5, wherein R⁵ is represented by a general formula (6): 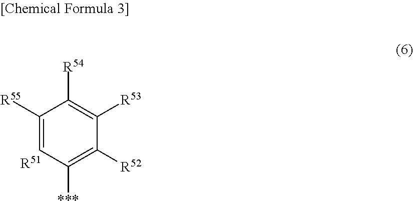
wherein *** represents a bonding moiety with a carbon atom, R⁵¹ and R⁵² are the same or different from each other, and are each selected from the group consisting of an alkyl group bonded to a substituent or no substituent, an aryl group bonded to a substituent or no substituent, and a heteroaryl group bonded to a substituent or no substituent, R⁵³ to R⁵⁵ are the same or different from each other, and are each selected from a hydrogen atom, an alkyl group, a cycloalkyl group, a heterocyclic group, an alkenyl group, a cycloalkenyl group, an alkynyl group, an aryl group, a heteroaryl group, a hydroxyl group, a thiol group, an alkoxy group, an alkylthio group, an aryl ether group, an aryl thioether group, a halogen, a cyano group, an aldehyde group, an acyl group, a carboxyl group, an ester group, an amide group, a sulfonyl group, a sulfonic acid ester group, a sulfonamide group, an amino group, a nitro group, a silyl group, and a ring structure with an adjacent group, and the groups that are substituted.

7. The pyrromethene metal complex according to claim 1, wherein R¹ in the general formula (1) or (2) is selected from an alkyl group bonded to a substituent or no substituent, an aryl group bonded to a substituent or no substituent, and a heteroaryl group bonded to a substituent or no substituent.

8. The pyrromethene metal complex according to claim 1, represented by any one of general formulae (7A) to (7M): 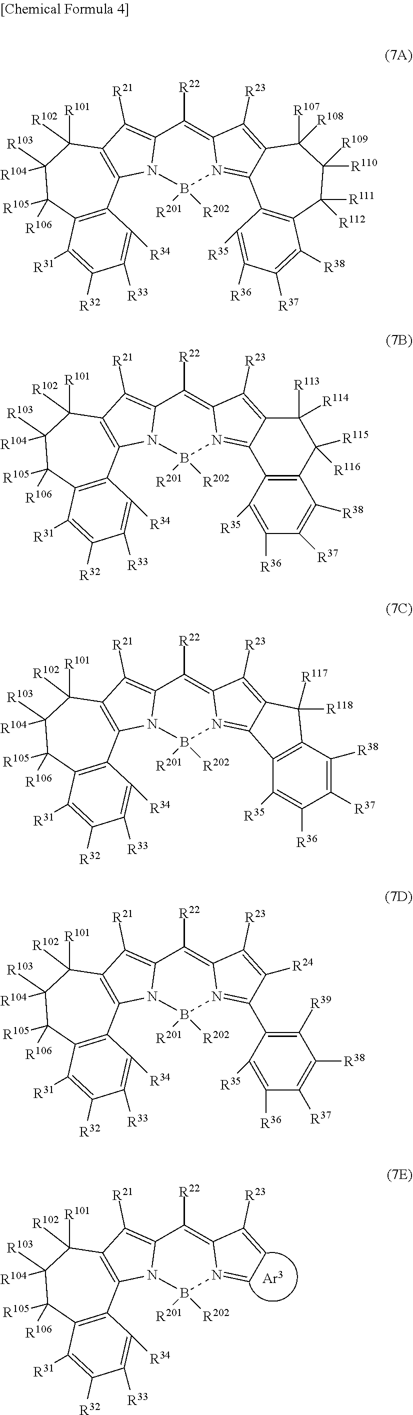 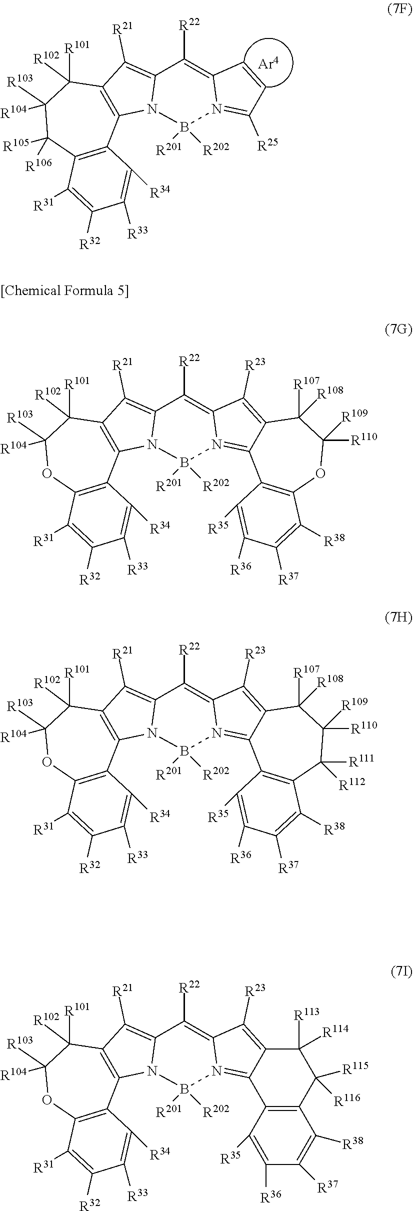 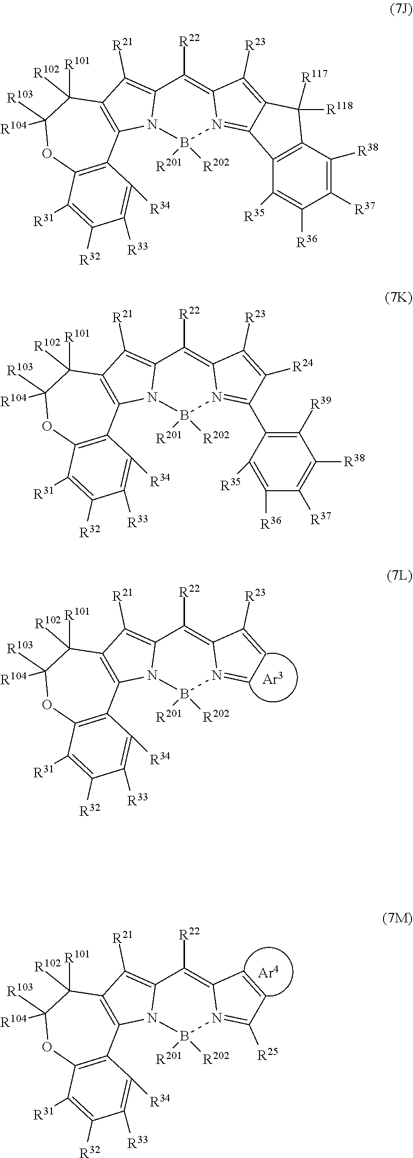
wherein R²¹ to R²⁵ are the same or different from each other, and are each selected from a hydrogen atom, an alkyl group, a cycloalkyl group, a heterocyclic group, an aryl group, and a heteroaryl group, and the groups that are substituted, R²¹ is not a hydrogen atom and not a halogen in a case that all of R¹⁰¹ to R¹⁰⁶ are a hydrogen atom,
R³¹ to R³⁹ are the same or different from each other, and are each selected from a hydrogen atom, an alkyl group, a cycloalkyl group, a heterocyclic group, an alkenyl group, a cycloalkenyl group, an alkynyl group, an aryl group, a heteroaryl group, a hydroxyl group, a thiol group, an alkoxy group, an alkylthio group, an aryl ether group, an aryl thioether group, a halogen, a cyano group, an aldehyde group, an acyl group, a carboxyl group, an ester group, an amide group, a sulfonyl group, a sulfonic acid ester group, a sulfonamide group, an amino group, a nitro group, a silyl group, a siloxanyl group, a boryl group, a phosphine oxide group, and a ring structure with an adjacent group, and the groups that are substituted,
R¹⁰¹ to R¹¹⁸ are the same or different from each other, and are each selected from a hydrogen atom, an alkyl group, a cycloalkyl group, a heterocyclic group, an alkenyl group, a cycloalkenyl group, an alkynyl group, an aryl group, a heteroaryl group, a hydroxyl group, a thiol group, an alkoxy group, an alkylthio group, an aryl ether group, an aryl thioether group, a halogen, a cyano group, an aldehyde group, an acyl group, a carboxyl group, an ester group, an amide group, a sulfonyl group, a sulfonic acid ester group, a sulfonamide group, an amino group, a nitro group, a silyl group, a siloxanyl group, a boryl group, a phosphine oxide group, and an oxo group, and the groups that are substituted, a ring structure is formed between any two substituents selected from R¹⁰¹ to R¹⁰⁶, between any two substituents selected from R¹⁰⁷ to R¹¹², between any two substituents selected from R¹¹³ to R¹¹⁶, or between R¹¹⁷ and R¹¹⁸, or no ring structure is formed between any two substituents selected from R¹⁰¹ to R¹⁰⁶, between any two substituents selected from R¹⁰⁷ to R¹¹², between any two substituents selected from R¹¹³ to R¹¹⁶, and between R¹¹⁷ and R¹¹⁸,
R²⁰¹ and R²⁰² are the same or different from each other, and are each selected from an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, and a cyano group, and the groups that are substituted,
Ar³ and Ar⁴ are the same or different from each other, and are each selected from an aromatic hydrocarbon ring bonded to a substituent or no substituent and an aromatic heterocyclic ring bonded to a substituent or no substituent.

9. (canceled)

10. A light-emitting element material comprising the pyrromethene metal complex according to claim 1.

11. A light-emitting element comprising:
an anode;
a cathode;
an emissive layer between the anode and the cathode; the emissive layer including the pyrromethene metal complex according to claim 1, wherein
the emissive layer emits light by electrical energy.

12. The light-emitting element according to claim 11, wherein the emissive layer includes a first compound and a second compound that is a dopant, and the second compound is the pyrromethene metal complex.

13. The light-emitting element according to claim 11, wherein the first compound is a thermally activated delayed fluorescent compound.

14. The light-emitting element according to claim 13, wherein the emissive layer further includes a third compound, and singlet excitation energy of the third compound is larger than singlet excitation energy of the first compound.

15. The light-emitting element according to claim 14, wherein the third compound includes two or more materials.

16. The light-emitting element according to claim 11, wherein at least two emissive layers are provided between the anode and the cathode, and at least one charge generation layer is interposed between the at least two emissive layers and at least one charge generation layer includes a phenanthroline derivative represented by a general formula (13): 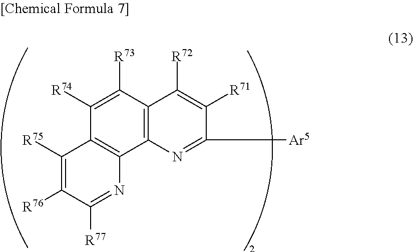
wherein Ar⁵ represents an arylene group substituted with two phenanthrolyl groups,
R⁷¹ to R⁷⁷ are the same or different from each other, and are each selected from a hydrogen atom, an alkyl group, a cycloalkyl group, a heterocyclic group, an aryl group, and a heteroaryl group.

17. (canceled)

18. The light-emitting element according to claim 11, wherein the light-emitting element is a top emission type organic electroluminescent element.

19. (canceled)

20. (canceled)
